(19) United States
(12) Patent Application Publication
(10) Pub. No.: US 2026/0309712 A1
(43) Pub. Date: Oct. 8, 2026

(54) COMPOSITIONS AND METHODS FOR DETECTION OF ABERRANT CDK5 EXPRESSION

(71) Applicants: ARIZONA BOARD OF REGENTS ON BEHALF OF THE UNIVERSITY OF ARIZONA, Tucson, AZ (US); THE UAB RESEARCH FOUNDATION, Birmingham, AL (US)

(72) Inventors: James BIBB, Phoenix, AZ (US); Priyanka GUPTA, Phoenix, AZ (US); Angela CARTER, Birmingham, AL (US)

(21) Appl. No.: 19/490,953

(22) PCT Filed: Jun. 10, 2024
(86) PCT No.: PCT/US2024/033266

§ 371 Date: Dec. 8, 2025

Related U.S. Application Data
(60) Provisional application No. 63/507,058, filed on Jun. 8, 2023.

(51) Int. Cl. G01N 33/573; C07K 16/40; G01N 33/575; G01N 33/68

(52) U.S. Cl. CPC G01N 33/573; C07K 16/40; G01N 33/57595; G01N 33/6893; C07K 2317/24; C07K 2317/34; C07K 2317/565; G01N 2333/4703; G01N 2333/91017; G01N 2440/14

(57) ABSTRACT

Improved antibodies for detection of phosphorylated RBL1, H1.5, LARP6, SUV39H1, and FAM53C have been developed. Assays using them to detect the biomarkers: P-T143 FAM53C, P-T202 LARP6, P-S988 RBL1, P-S17 H1.5, and P-S391 SU-V39H1, and diagnostic and prognostic applications stemming therefrom are also provided.

### CROSS-REFERENCE TO RELATED APPLICATIONS

This application claims the benefit of and priority to U.S. Application No. 63/507,058 filed Jun. 8, 2023, the contents of which are incorporated by reference herein in their entirety.

### REFERENCE TO THE SEQUENCE LISTING

The Sequence Listing XML submitted as a file named “UA23-214PCT” and having a size of 78,203 bytes is hereby incorporated by reference pursuant to 37 C.F.R. § 1.834(c)(1).

### FIELD OF THE INVENTION

The field of the invention is general compositions and methods for detection of aberrant Cdk5 activity in biological samples such as tumor samples.

### BACKGROUND OF THE INVENTION

The “one-size-fits-all” therapeutic approach has been unsuccessful in treating majority of cancer patients, causing a spike in global incidence rates of neuroendocrine tumors (NETs) with the highest escalation rate in North America and Canada. The incidence of NETs in the USA alone increased by 6 folds showing a current prevalence of ~170,000 patients. According to the World Cancer Research Fund International, colorectal cancer and melanoma are the third and fifth most common types of cancer worldwide. Estimations indicate 1.8 million cases of colorectal cancer and 486,000 cases of melanoma in 2020. An early detection and treatment of these catastrophic cancers can greatly improve survival rates. Challenges like heterogeneity in the clinical presentation of tumors, indolent-to-progressive disease courses, frequent metastasis, and ineffective existing treatments combat adequate management of these tumors.

Cdk5 has emerged as an important instigator of tumorigenic signaling in several types of NETs including pancreatic, adrenal, thyroid, and gastrointestinal tumors. Non-neuroendocrine malignancies such as colorectal cancer and melanomas are also afflicted with pathological activation of Cdk5 signaling. Cdk5 has been shown to promote tumor growth and invasion by regulating the activity of proteins involved in metabolic reprogramming, cytoskeletal remodeling, and epithelial-mesenchymal transition.

For example, medullary thyroid carcinoma (MTC) is derived from calcitonin-secreting parafollicular neuroendocrine (NE) cells. These tumors occur as sporadic or hereditary forms with an incidence rate of approximately 75% and 25%, respectively. MTC patients present clinically heterogeneous disease courses ranging from indolent to highly aggressive. The survival rate of 10-years varies from 100% (stage I) to 21% (stage IV) [1]. MTC accounts for 5-10% of all thyroid malignancies and is frequently associated with germline mutations in the RET proto-oncogene. Patients may also harbor somatic mutations in HRAS, KRAS, or NRAS [2]. The 5-year survival rate is ~40% in patients with metastatic disease where tumors can spread to the cervical lymph nodes, or distant sites such as bones, lungs, liver, and brain [3-5]. Surgery is the only curative therapy for MTC, but resection of isolated metastases or other newer treatments have shown promise [6, 7]. However, a proper therapeutic regimen for aggressive, recurrent, and metastatic disease is still in abeyance.

RET mutations, serum calcitonin, and carcinoembryonic antigen (CEA) are known prognostic markers for MTCs. However, risk stratification based on serum biomarkers, namely calcitonin, CEA, carbohydrate antigen 19.9, or Ki67 expression has proven inefficient in identifying aggressive phenotypes, patients requiring immediate treatment, or resistance to existing therapeutic modalities [8]. The continuous global increase in MTC incidence corresponds with spiking mortality rates. Furthermore, epidemiological evidence from the past three decades has not indicated improvement in MTC diagnosis or overall patient survival. Lack of adequate predictive biomarkers, inconsistent long-term prognostic factors, and poor identification of aggressive phenotype attributed to the indolent course of the disease. Likewise, the unpredictable clinical behavior of patients triggers the overarching need for reliable biomarkers to detect aggressive forms of tumors. Thus, there remains a need for improved means of diagnosing and treating Cdk5+ cancers such as MTC.

It is an object of the invention to provided compositions and methods thereof for diagnosing and treating Cdk5+ cancer such as MTC.

### SUMMARY OF THE INVENTION

Improved antibodies for detection of phosphorylated RBL1, H1.5, LARP6, SUV39H1, and FAM53C have been developed. Assays using them to detect the biomarkers: P-T143 FAM53C, P-T202 LARP6, P-S988 RBL1, P-S17 H1.5, and P-S391 SUV39H1, and diagnostic and prognostic applications stemming therefrom are also provided.

For example, antibodies or other molecules including an antigen binding domain of an antibody that immunospecifically binds to the epitope: CSIYI-pS-PHKN (SEQ ID NO:1); CAPVEK-pS-PAK (SEQ ID NO:14); CALA-pT-PQKNGRV (SEQ ID NO:27); CLAGLPG-pS-PKKRVR (SEQ ID NO:40); or CAPSKLW-pT-PIKH (SEQ ID NO:53) are disclosed. The antigen binding domain typically includes six complementarity-determining regions (CDRs), wherein the CDRs includes one, two, three, four, five, or six consensus CDRs of the CDRs of: anti-RBL1 antibody RBL1-1; anti-H1.5 antibody H1-5-1; anti-LARP6 antibody LARP6-1; anti-SUV39H1 antibody SUV39H1-1; anti-FAM53C antibody FAM53C-1; or a variant thereof with at least 70% sequence identity thereto. In some embodiments, the antibodies and molecules include the heavy and/or light chain variable region(s) of anti-RBL1 antibody RBL1-1; anti-H1.5 antibody H1-5-1; anti-LARP6 antibody LARP6-1; anti-SUV39H1 antibody SUV39H1-1; anti-FAM53C antibody FAM53C-1; or a variant thereof with at least 70% sequence identity thereto.

The antibodies can be intact antibodies or functional antibody fragment or fusion proteins. Examples of functional fragments or fusion proteins are selected from Fab fragments, F(ab′)2 fragments, Fab′ fragments, Fv fragments, recombinant IgG (rIgG) fragments, single chain antibody fragments such as single chain variable fragments (scFv), and single domain antibodies such as sdAb, sdFv, and nanobody fragments. The antibodies can be intrabodies, peptibodies, chimeric antibodies, fully human antibodies, humanized antibodies, heteroconjugate antibodies, and multispecific antibodies such as bispecific antibodies, diabodies, triabodies, tetrabodies, tandem di-scFv, and tandem tri-scFv. The antibodies can be IgM, IgE, IgA, IgD, or IgG optionally an IgG1, IgG2, IgG3, or IgG4. Any of the antibodies can further include a detectable label (substance) and/or a conjugated toxin, drug, receptor, enzyme, or receptor ligand.

Methods of detecting Cdk5 are also provided. An exemplary method includes detecting the level of phosphorylation of one or more of P-T143 FAM53C, P-T202 LARP6, P-S988 RBL1, P-S17 H1.5, and P-S391 SUV39H1 by contacting a biological sample with one or more of the disclosed antibodies, and detecting binding between the antibody or antibodies and the one or more of FAM53C, LARP6, RBL1, H1.5, and SUV39H1. The method can further include determining that the sample includes aberrant Cdk5 activity if the level of detected binding is higher in the biological sample than in a control. Exemplary means of detecting the antibody or antibodies include, but are not limited to immunoassays, such as immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, or immuno-chromatography assays as well as immunohistochemistry, Western blotting, surface plasmon resonance, flow cytometry (FACS) analysis, and biochips.

In some embodiments, the biological sample is cells or a cell lysate or a fraction thereof. The cells or cell lysate or fraction thereof can be derived from a biopsy from a subject. The biopsy can contain or be suspected of containing tumor cells. The biopsy can be a tumor biopsy.

Diagnostic methods are also provided. For example, diagnosing a subject with a Cdk5-related disease or disorder can include detecting aberrant Cdk5 according to the disclosed methods. In some embodiments, the Cdk5-related disease or disorder is cancer, diabetes, or obesity. In some embodiments, the cancer is a neuroendocrine cancer, colorectal cancer, melanoma, lung cancer, head and neck squamous cell carcinoma, hepatocellular carcinoma (HCC), pancreatic cancer, or breast cancer. In more specific embodiments, the neuroendocrine cancer is Medullary Thyroid Carcinoma (MTC), pancreatic, adrenal, thyroid, or gastrointestinal cancer.

Methods of treating diagnosed subjects are also provided and can be used separately or in conjunction with the diagnostic methods. In some embodiments, the treatment includes a therapy effective for treating diseases and disorders characterized by aberrant Cdk5 activity. Exemplary treatments include dinaciclib (SCH727965), a proteosome inhibitor optionally Bortezomib (Velcade), or a functional nucleic acid that reduces expression of the Cdk5 gene, mRNA, or protein, optionally wherein the functional nucleic acid is siRNA, miRNA, RNAi, or shRNA.

### BRIEF DESCRIPTION OF THE DRAWINGS

FIGS. 1A-1G illustrate the generation of transgenic mouse models of MTC. FIG. 1A is a schematic showing tetracycline controlled bitransgenic system where a neuroendocrine cell-specific promoter linked to the tetracycline transactivator (tTA) activates Tet-Operon driving p25-GFP expression. The resultant Cdk5-p25GFP interaction promotes neoplastic transformation. FIGS. 1B-1C are schematics showing the induction of p25GFP in doxycycline-controlled bitransgenic models driven by NSE and CGRP promoters. FIG. 1D are images showing gross anatomy of tumors in NSE-p25GFP and CGRP-p25GFP models (upper panel); histopathological neuroendocrine features characterized by H & E (bottom panel), scale; 50 μm, tr; trachea. FIG. 1E is a Tumor volume growth curves of NSE (growing n=4; arrested n=5) and CGRP (growing n=3; arrested n=5) mice showing weekly measurements of tumor volume under Dox Off vs. Dox On conditions (left); and a bar graph showing tumor volume fold change of growing tumors (G; 14 weeks Dox Off) relative to arrested (A; 14 weeks Dox On) (right); values are mean±SD, *p<0.05 compared by Student's t-test. FIGS. 1F and 1G are representative immunostains comparing expression of p25GFP and Chromogranin A (ChrA) in NSE (FIG. 1F) and CGRP (FIG. 1G) mice tissues extracted from growing and arrested tumors. A version of NSE-p25GFP MTC tumors (FIG. 1D, left) was previously published [10] and shown here at a different magnification for comparison.

FIGS. 2A-2G illustrates the mutational landscape of mouse tumor models. FIG. 2A is a map showing mutation profile of growing (Dox Off or p25OE) NSE (n=4) and CGRP (n=3) mouse tumors across chromosomes. FIG. 2B is a plot of variant classification by type; frequency of variant (y-axis), colors denote types of variation. FIG. 2C is a plot of variant type presented as SNP (single nucleotide polymorphism), INS (insertion), DNP (double nucleotide polymorphism), DEL (deletion), TNP (triple nucleotide polymorphism); frequency (y-axis). FIG. 2D is a UpSet plot showing counts of unique or overlapping mutated genes in NSE and CGRP models. FIG. 2E is a bar chart displaying enriched pathways associated with altered genes in NSE-p25OE mice. FIG. 2F is a bar chart displaying enriched pathways in CGRP-p25OE mice. FIG. 2G is a bar chart comparing enriched pathways common in -NSE and -CGRP p25OE mice. Significant functions are shown where p-value<0.01.

FIGS. 3A-3D illustrate comparison of mouse and human tumors mutational profile. FIG. 3A is a Venn diagram showing overlapping genes between NSE-p25OE mice and human tumors [35]. FIG. 3B is a GO term enrichment analysis shows biological processes and pathways associated with the altered genes shared by NSE-p25OE mice and human tumors. FIG. 3C is a Venn diagram showing overlapping mutated genes in CGRP-p25OE mice and human tumors [35]. FIG. 3D is a GO analysis of overlapping genes shared by CGRP-p25OE mice and human tumors. Bars sorted by p-value ranking; p-value cut-off<0.05; GO, Gene Ontology.

FIGS. 4A-4G illustrate transcriptomic analysis of mouse models. FIG. 4A is principal component analysis of RNA-seq reads in NSE-p25OE (n=4 growing; n=5 arrested) and CGRP-p25OE mice (n=5 growing; n=5 arrested). FIG. 4B is a volcano plot presenting differentially expressed genes (DEGs) in growing vs. arrested tumors of NSE-p25OE and CGRP-p25OE models. Red=upregulated DEGs; Green=downregulated DEGs; log₂ FC>1.5. FIG. 4C is a UpSet plot summarize unique and overlapping DEGs in NSE and CGRP p25OE models. FIG. 4D-4G are illustrations showing visualization of enrichment network of DEGs up and downregulated (FIGS. 4D-4E) in NSE-p25OE mice, and (FIGS. 4F-4G) in CGRP-p25OE mice. Cluster annotation is indicated by color code, nodes with the same color are closely spaced and associated with the same cluster, clusters were labeled manually; enriched terms with a similarity score of >0.3 are connected by edges.

FIGS. 5A-5J illustrate the impact of genetic alterations on mRNA expression. FIG. 5A is an UpSet plot depicts the overlap between mutations and corresponding gene expression in NSE-p25OE mice. FIGS. 5B and 5C are bar graphs showing pathway visualization of overlapping genes afflicted both with mutations and changes in mRNA expression: where mRNA was up- (FIG. 5B) or down-regulated (FIG. 5C) in NSE-p25OE mice. FIG. 5D is a series of violin plots comparing mRNA reads of mutated genes involved in spindle assembly and Notch signaling. FIG. 5E is a series of violin plots comparing mRNA reads of mutated genes enriched in cancer-associated proliferative signaling (G, growing; A, arrested tumors). FIG. 5F is a bar graph showing intersection size of mutated genes and corresponding mRNAs in CGRP-p25OE mice. FIGS. 5G and 5H are bar graphs showing functional enrichment of overlapping mutated genes where mRNA was either up- (FIG. 5G) or down-regulated (FIG. 5H) in the CGRP-p25OE mice. FIGS. 5I and 5J are each a pair of plots showing mRNA reads of mutated genes associated with metabolic pathways and cell migration (FIG. 5I), or mRNA reads of mutated genes involved in mTOR signaling (FIG. 5J).

FIGS. 6A-6E illustrate efficacy of monoclonal antibodies for detection of aberrant Cdk5 activity. FIG. 6A is a graph showing cell proliferation in mouse MTC cells under p25OE vs. p25OFF conditions, n=3, Mean±SD, *** p<0.001. FIG. 6B is a series of images showing immunocytochemical analysis of Cdk5-dependent phosphorylations in mouse MTC cells as indicated, scale; 70 μm. FIG. 6C is a series of images of immunohistochemical staining of Cdk5 phosphorylations in growing (p25OE) and arrested (p25OFF) tumor sections derived from NSE/CGRP models, scale; 50 μm. FIGS. 6D and 6E are each a series of images showing immunofluorescent (FIG. 6D) and immunohistochemical micrographs (FIG. 6E) of Cdk5 phosphosites in human TT cells and patient tumor sections, respectively (n=5), scale; 50 μm.

FIGS. 7A-7D illustrate histological evaluation of aberrant Cdk5 activity in human tissue microarray. FIGS. 7A and 7B are representative immunohistochemical micrographs showing Cdk5 phosphosites viz. P-RBL1 (FIG. 7A) and P-LARP6 (FIG. 7B) in tissue microarray sections of MTC tumors. magnification; 4× (scale, 200 μm), 20× (scale, 50 μm). FIGS. 7C and 7D are each a series of plots showing quantification presented as the optical density for (FIG. 7C) P-RBL1 (TMA 1-3), and (FIG. 7D) P-LARP6 (TMA 1-3). TMA 1: Nor=5, tumor=25; TMA 2: Nor=5, tumor=12; TMA 3: n=5, tumor=11. *p<0.05, **p<0.01, ***p<0.001; values compared using Student's t-test with Welch's correction.

FIGS. 8A-8B illustrate enrichment analysis of mutated genes in mouse tumors. KEGG and GO enrichment in (A) NSE-p25OE and, (B) CGRP-p25OE mice. y-axis=pathway description; x-axis=fold enrichment. Analysis performed in Shiny GO 0.76.2; the bubble size indicates the number of genes, and the bar color code signifies the corrected p-value as indicated. GO: Gene Ontology (biological process); KEGG: Kyoto Encyclopedia of Genes and Genomes.

FIGS. 9A-9C illustrate mitotic cell cycle pathways shared by NSE mouse and human tumors. FIG. 9A is a UpSet plot showing intersecting mutated genes in mouse and human tumors. FIG. 9B is a Lollipop chart and FIG. 1C is a network visualization summarize significantly enriched GO terms common in NSE and human tumors. In a network diagram, two nodes are connected if they share >20% of their genes, FDR cutoff=0.05.

FIGS. 10A-10B illustrate metabolic pathways shared by CGRP mouse and human tumors. FIG. 10A is a plot showing counts of unique and overlapping genes between mouse and human tumors. FIG. 10B is a pathway and process enrichment plot and FIG. 10C is a network tree of significantly enriched gene sets common in CGRP-p25OE mouse and human MTC, FDR cutoff=0.05.

FIGS. 11A-11B illustrates enrichment of transcription factors in NSE-p25OE mice. FIG. 11A is a bar graph showing putative regulators of DEGs in NSE-p25OE mice determined via TRRUST database [73]. FIG. 11B is a bar graph summarizing the enriched transcription factor in CGRP-p25OE mice. TRRUST database revealed putative regulators of DEGs in CGRP-p25OE mice. Terms with a p-value<0.01; minimum count of 3, enrichment factor>1.5 [74].

FIG. 12 illustrates detection of Cdk5 activity in NSE and CGRP tumors. Immunoblots show expression levels of p25GFP, P-RBL1, and P-H1.5 in tissue lysates extracted from growing (p25OE) vs. arrested (p25OFF) tumors. Polyclonal antibodies were used for probing the indicated phosphosites; mMTC=mouse MTC.

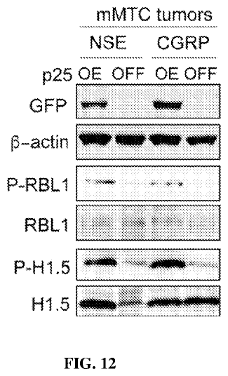

FIGS. 13A-13E illustrate characterization of recombinant phospho-specific mcAbs in mouse tumors. Comparative analysis of Cdk5 phosphorylation state-specific polyclonal antibodies (pcAb) vs. recombinant mcAbs. Lysates from growing (“G”) and arrested (“A”) tumors were analyzed via immunoblotting using Abs as indicated. Phospho-sites probed were (FIG. 13A) phospho-Ser988 RBL1, (FIG. 13B) phospho-Thr202 LARP6, (FIG. 13C) phospho-Ser17 Histone H1.5, (FIG. 13D) phospho-Ser391 SUV39H1, and (FIG. 13E) phospho-Thr143 FAM53C.

### DETAILED DESCRIPTION OF THE INVENTION

### I. Definitions

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multispecific antibodies (e.g., bispecific antibodies), and antibody fragments so long as they specifically bind the target antigen. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. The term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia, C. et al. (1987) “Canonical Structures For The Hypervariable Regions Of Immunoglobulins,” J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies of the invention). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass. An “antibody fragment” or “antigen binding fragment” of an antibody is defined as at least a portion of the variable region of the immunoglobulin molecule that binds to its target, i.e., the antigen binding region (also antigen binding domain). In one embodiment it specifically covers single antibodies and clones thereof and anti-antibody compositions with polyepitopic specificity. The antibody of the present methods and compositions can be monoclonal or polyclonal. An antibody can be in the form of an antigen binding antibody fragment including a Fab fragment, F(ab′)₂ fragment, a single chain variable region, and the like. Fragments of intact molecules can be generated using methods well known in the art and include enzymatic digestion and recombinant means. Thus, the “fragment” may be a recombinant protein, e.g., a fusion protein.

As used herein, any form of the “antigen” can be used to generate an antibody that is specific for the target antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least a portion of the extracellular domain of the target antigen of interest.

The antibodies or antigen binding fragments thereof provided herein may be conjugated to a “bioactive agent.” As used herein, the term “bioactive agent” refers to any synthetic or naturally occurring compound that binds the antigen and/or enhances or mediates a desired biological effect.

In one embodiment, the binding fragments useful in the present invention are biologically active fragments. As used herein, the term “biologically active” refers to an antibody or antibody fragment that is capable of binding the desired the antigenic epitope and directly or indirectly exerting a biologic effect.

“Bispecific” antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison et al., Proc. Natl. Acad. Sci. USA 81: 6851-6855 (1984)).

The term “specifically binds” or “immuno-specifically binds” refers to the binding of an antibody to its cognate antigen while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The term “monoclonal antibody” or “mAb” refers to an antibody obtained from a substantially homogeneous population of antibodies, i.e., the individual antibodies within the population are identical except for possible naturally occurring mutations that may be present in a small subset of the antibody molecules.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

Hyperactive Cdk5 plays an important role in MTC progression [10], and is believed to play a role in other diseases and conditions as well. A positive correlation was demonstrated between Cdk5 and its downstream targets in human MTC. Protein phosphorylation substrates of this aberrantly active kinase included P-T143 FAM53C, P-T202 LARP6, P-S988 RBL1, P-S17 H1.5, and P-S391 SUV39H1 [48]. In the experiments below, both NSE/CGRP models manifest aberrant Cdk5 activation as indicated by increased Cdk5-dependent phosphorylation in growing (p25OE) versus arrested tumors (p25OFF) support the conclusion that these phosphosites can serve as biomarkers for the detection of Cdk5-driven human tumors. However, existing polyclonal antibodies to these Cdk5 targets were raised against short phospho-peptide epitopes that were limited in specificity and often detected cross-reactive proteins harboring similar phosphorylation site motifs. Thus, the existing reagents for detecting these biomarkers are insufficient to use for detection and diagnostic purposes with high confidence. To solve this problem, improved antibodies have been developed, as well as assays using them to detect the biomarkers, and diagnostic and prognostic applications.

Disclosed are antibodies and their antigen binding fragments and other molecules that are capable of immunospecifically binding phosphorylated proteins downstream of aberrantly active Cdk5. More particularly, antibodies and their antigen binding fragments and other molecules capable of immunospecifically binding P-T143 FAM53C, P-T202 LARP6, P-S988 RBL1, P-S17 H1.5, and P-S391 SUV39H1 are provided.

As discussed elsewhere herein in more details, in non-limiting embodiments, such molecules can be, for example, a monoclonal antibody, a human antibody, a chimeric antibody or a humanized antibody, or a fragment thereof, and fusion proteins formed therefrom. The antibodies and antigen binding fragments can be monospecific, bispecific, trispecific or multispecific.

### A. Variable Region Sequences

### 1. RBL1

Polypeptides that selectively bind RB transcriptional corepressor like 1 (RBL1) are provided. RBL1 regulates the cell cycle, and proliferation by modulating chromatin structure. See, e.g., RB transcriptional corepressor like 1 [Homo sapiens (human)], NCBI Gene ID: 5933.

In some embodiments, the polypeptide specifically binds the epitope: CSIYI-pS-PHKN (SEQ ID NO:1).

The molecules and antibodies that bind to RBL1 can include an antigen binding domain that includes six CDRs, wherein the CDRs include at least one, two, three, four, five, or six consensus CDRs of the CDRs of anti-RBL1 antibody RBL1-1. In some embodiments, the molecule or antibody includes a heavy and/or light chain variable region of antibody RBL1-1. In some embodiments, the CDRs and/or the heavy and light chain variable regions are in the same orientation as antibody RBL1-1.

An exemplary consensus amino acid sequence for the mature heavy chain variable region of RBL1-1 is:

(SEQ ID NO: 2)
QSLEESGGRLVTPGTPLKLTCTVSGFSLSDYNVGWVRQAPGKGLEWIGI
MNIGISTWYASWAKGRFTISRTSTTVDLKMTSLTTEDTATYFCARGFSR
NSYDIWGPGTLVTVSS
CDR-H1:
(SEQ ID NO: 4)
DYNVG
CDR-H2:
(SEQ ID NO: 5)
IMNIGISTWYASWAKG
CDR-H3:
(SEQ ID NO: 6)
GFSRNSYDI

An exemplary consensus amino acid sequence for the mature light chain variable region of RBL1-1 is:

(SEQ ID NO: 3)
AVLTQTPSPVSAAVGGTVTIKCQSSQSVVKNNYLSWYQQKPGQPPKLLI
YETSKLASGVPSRFKGSGSGTQFTLTISDVQCDDAATYYCAGGYSSISD
TTFGGGTEVVVK,
CDR-L1:
(SEQ ID NO: 7)
QSSQSVVKNNYLS
CDR-L2:
(SEQ ID NO: 8)
ETSKLAS
CDR-L3:
(SEQ ID NO: 9)
AGGYSSISDTT

See also SEQ ID NOS:10-13, which provide the amino acid and nucleic acid sequences of full heavy and light chains of RBL1-1, including the cleavable signal sequences and the constant regions.

### 2. H1.5

Polypeptides that selectively bind H1.5 are provided. H1.5 is a linker histone that facilitates chromatin compaction. See, e.g., H1.5 linker histone, cluster member [Homo sapiens (human)], NCBI Gene ID: 3009.

In some embodiments, the polypeptide specifically binds the epitope: CAPVEK-pS-PAK (SEQ ID NO:14).

The molecules and antibodies that bind to H1.5 can include an antigen binding domain that includes six CDRs, wherein the CDRs include at least one, two, three, four, five, or six consensus CDRs of the CDRs of anti-H1.5 antibody H1-5-1. In some embodiments, the molecule or antibody includes a heavy and/or light chain variable region of antibody H1-5-1. In some embodiments, the CDRs and/or the heavy and light chain variable regions are in the same orientation as antibody H1-5-1.

An exemplary consensus amino acid sequence for the mature heavy chain variable region of H1-5-1 is:

(SEQ ID NO: 15)
QSVEESGGRLVTPGTPLTLTCTVSGIDLSSNVMMWVRQAPGKGLEYIGI
ITNSGIRYYASWAKGRFTISKTSTTVDLKITSPTTEDTATYFCARGAPN
TGNIWGPGTLVTVSL
CDR-H1:
(SEQ ID NO: 17)
SNVMM
CDR-H2:
(SEQ ID NO: 18)
IITNSGIRYYASWAKG
CDR-H3:
(SEQ ID NO: 19)
GAPNTGNI

An exemplary consensus amino acid sequence for the mature light chain variable region of H1-5-1 is:

(SEQ ID NO: 16)
QVLTQTASSVSAAVGGTVTINCQSSQSVYDNNYLSWYQQKPGQPPKLLI
YQASKLASGVPSRFKGSGSGTQFTLTISDLECDDAVTYYCAGAYFGNIY
TFGGGTEVVVK
CDR-L1:
(SEQ ID NO: 20)
QSSQSVYDNNYLS
CDR-L2:
(SEQ ID NO: 21)
QASKLAS
CDR-L3:
(SEQ ID NO: 22)
AGAYFGNIYT

See also SEQ ID NOS:23-26, which provide the amino acid and nucleic acid sequences of full heavy and light chains of H1-5-1, including the cleavable signal sequences and the constant regions.

### 3. LARP6

Polypeptides that selectively bind La Ribonucleoprotein 6 (LARP6) are provided. LARP6 is a translational regulator shuttles between cytoplasm and nucleus promotes nucleic acid binding. See, e.g., LARP6 La ribonucleoprotein 6, translational regulator [Homo sapiens (human)], NCBI Gene ID: 55323.

In some embodiments, the polypeptide specifically binds the epitope: CALA-pT-PQKNGRV (SEQ ID NO:27).

The molecules and antibodies that bind to LARP6 can include an antigen binding domain that includes six CDRs, wherein the CDRs include at least one, two, three, four, five, or six consensus CDRs of the CDRs of anti-LARP6 antibody LARP6-1. In some embodiments, the molecule or antibody includes a heavy and/or light chain variable region of antibody LARP6-1. In some embodiments, the CDRs and/or the heavy and light chain variable regions are in the same orientation as antibody LARP6-1.

An exemplary consensus amino acid sequence for the mature heavy chain variable region of LARP6-1 is:

(SEQ ID NO: 28)
QSLEESGGGLVQPEGSLTLTCKASGFSFSSGYYMCWVRQAPGKGLEWSG
CISARSGRTYYATWAKGRFTISKTSSTTVTLQVTSLTAADTATYFCARG
NRFVSSSGDSMWGPGTLVTVSS
CDR-H1:
(SEQ ID NO: 30)
SGYYMC
CDR-H2:
(SEQ ID NO: 31)
CISARSGRTYYATWAKG
CDR-H3:
(SEQ ID NO: 32)
GNRFVSSSGDSM

An exemplary consensus amino acid sequence for the mature light chain variable region of LARP6-1 is:

(SEQ ID NO: 29)
DPVLTQTPSPVSAAVGGTVTINCQASQSVEFSNNQLAWFQQKPGQPPKQ
LIYGASTLASGVSSRFKGSGYGTRFTLTISDVQCDDTATYYCLGEFTCS
SVDCNAFGGGTEVVVE,
CDR-L1:
(SEQ ID NO: 33)
QASQSVFSNNQLA
CDR-L2:
(SEQ ID NO: 34)
GASTLAS
CDR-L3:
(SEQ ID NO: 35)
LGEFTCSSVDCNA

See also SEQ ID NOS:36-39, which provide the amino acid and nucleic acid sequences of full heavy and light chains of LARP6-1, including the cleavable signal sequences and the constant regions.

### 4. SUV39H1

Polypeptides that selectively bind SUV39H1 are provided. SUV39H1 is a histone lysine methyltransferase. See, e.g., SUV39H1 histone lysine methyltransferase [Homo sapiens (human)] NCBI Gene ID: 6839.

In some embodiments, the polypeptide specifically binds the epitope: CLAGLPG-pS-PKKRVR (SEQ ID NO:40).

The molecules and antibodies that bind to SUV39H1 can include an antigen binding domain that includes six CDRs, wherein the CDRs include at least one, two, three, four, five, or six consensus CDRs of the CDRs of anti-SUV39H1 antibody SUV39H1-1. In some embodiments, the molecule or antibody includes a heavy and/or light chain variable region of antibody SUV39H1-1. In some embodiments, the CDRs and/or the heavy and light chain variable regions are in the same orientation as antibody SUV39H1-1.

An exemplary consensus amino acid sequence for the mature heavy chain variable region of SUV39H1-1 is:

(SEQ ID NO: 41)
QSVEESGGRLVTPGTPLTLTCTVSGFSLSTYHMCWVRQAPGKGLEYIGM
INRRAITSYASWAKGRFTISKTSTTVDLKITSPTTEDTATYFCARYSSG
NDFDADIWGPGTLVTVSL
CDR-H1:
(SEQ ID NO: 43)
TYHMC
CDR-H2:
(SEQ ID NO: 44)
MINRRAITSYASWAKG
CDR-H3:
(SEQ ID NO: 45)
YSSGNDFDADI

An exemplary consensus amino acid sequence for the mature light chain variable region of SUV39H1-1 is:

(SEQ ID NO: 42)
AAVLTQTPSPVSAAVGGTVTISCQSSKSVYDRNLLSWFQQKPGQPPKLL
IYKASTLASGVPSRFKGSGSGTQFTLTISDVQCDDAATYYCAGGYSGTS
DAYPFGGGTEVVVK,
CDR-L1:
(SEQ ID NO: 46)
QSSKSVYDRNLLS
CDR-L2:
(SEQ ID NO: 47)
KASTLAS
CDR-L3:
(SEQ ID NO: 48)
AGGYSGTSDAYP

See also SEQ ID NOS:49-52, which provide the amino acid and nucleic acid sequences of full heavy and light chains of SUV39H1-1, including the cleavable signal sequences and the constant regions.

### 5. FAM53C

Polypeptides that selectively bind family with sequence similarity 53 member C (FAM53C) are provided. FAM53C bind to a transcriptional regulator that modulates cell proliferation. See, e.g., FAM53C family with sequence similarity 53 member C [Homo sapiens (human)] NCBI Gene ID: 51307.

In some embodiments, the polypeptide specifically binds the epitope: CAPSKLW-pT-PIKH (SEQ ID NO:53).

The molecules and antibodies that bind to FAM53C can include an antigen binding domain that includes six CDRs, wherein the CDRs include at least one, two, three, four, five, or six consensus CDRs of the CDRs of anti-FAM53C antibody FAM53C-1. In some embodiments, the molecule or antibody includes a heavy and/or light chain variable region of antibody FAM53C-1. In some embodiments, the CDRs and/or the heavy and light chain variable regions are in the same orientation as antibody FAM53C-1.

An exemplary consensus amino acid sequence for the mature heavy chain variable region of the FAM53C-1 is:

(SEQ ID NO: 54)
QSLEESGGDLVKPGASLTLTCKGSGFSFTIRYNICWVRQAPGKGLEWIA
CVNSAYASWAKGRFTISKTSSTTVTLQMTSLTAADTATYFCVRYVDSRY
YGVDLWGPGTLVTVSS
CDR-H1:
(SEQ ID NO: 56)
IRYNIC
CDR-H2:
(SEQ ID NO: 57)
CVNSAYASWAKG
CDR-H3:
(SEQ ID NO: 58)
YVDSRYYGVDL

An exemplary consensus amino acid sequence for the mature light chain variable region of FAM53C-1 is:

(SEQ ID NO: 55)
QVLTQTPSSVSAALGGTVTISCQSSESVYKNNYLSWYQQKPGQPPKLLI
YGASTLASGVPSRFKGSGSGTQFTLTISSVQCDDAATYYCQGGYRGDYS
SGDGILFGGGTEVVVK ,
CDR-L1:
(SEQ ID NO: 59)
QSSESVYKNNYLS
CDR-L2:
(SEQ ID NO: 34)
GASTLAS
CDR-L3:
(SEQ ID NO: 60)
QGGYRGDYSSGDGIL

See also SEQ ID NOS:61-64, which provide the amino acid and nucleic acid sequences of full heavy and light chains of SUV39H1-1, including the cleavable signal sequences and the constant regions.

### B. Constant Regions

In some embodiments, the disclosed antibodies and molecule include at least a portion of an immunoglobulin constant region (Fc), for example, that of a human immunoglobulin. The constant domains can be selected with respect to the proposed function of the antibody. In some embodiments, the constant domains of the antibodies are (or include) human IgA, IgD, IgE, IgG or IgM domains. In a specific embodiment, human IgG constant domains, especially of the IgG1 and IgG3 isotypes are used.

In some embodiments, the antibodies and other molecules contain both the light chain as well as at least the variable domain of a heavy chain. In some embodiments, the antibody or other molecule further includes one or more of the CH1, hinge, CH2, CH3, and CH4 regions of the heavy chain. The antibody can be selected from any class of immunoglobulins, including IgM, IgG, IgD, IgA and IgE, and any isotype, including IgG₁, IgG₂, IgG₃ and IgG₄.

The heavy chain constant region of the antibodies utilized in the examples below is:

(SEQ ID NO: 65)
GQPKAPSVFPLAPCCGDTPSSTVTLGCLVKGYLPEPVTVTWNSGTLTNG
VRTFPSVRQSSGLYSLSSVVSVTSSSQPVTCNVAHPATNTKVDKTVAPS
TCSKPMCPPPELLGGPSVFIFPPKPKDTLMISRTPEVTCVVVDVSQDDP
EVQFTWYINNEQVRTARPPLREQQFNSTIRVVSTLPIAHQDWLRGKEFK
CKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQ
RGDVFTCSVMHEALHNHYTQKSISRSPGK.

The light chain constant regions of the antibodies utilized in the examples below are:

(SEQ ID NO: 66)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC,
or
(SEQ ID NO: 67)
GDPVAPTVLIFPPSADLVATGTVTIVCVANKYFPDVTVTWEVDGTTQTT
GIENSKTPQNSADCTYNLSSTLTLTSTQYNSHKEYTCKVTQGTTSVVQS
FNRGDC.

### C. Antibody Derivatives, Variants, and Conjugates

The disclosure particularly contemplates the production and use of “derivatives” of any of the above-described antibodies and their antigen-binding fragments. The term “derivative” refers to an antibody or antigen-binding fragment thereof that immunospecifically binds to an antigen but which includes, one, two, three, four, five or more amino acid substitutions, additions, deletions or modifications relative to a “parental” (or wild-type) molecule.

Thus, in some embodiments, derivative is or includes a variant amino acid sequence. For example, provided are variants of SEQ ID NOS:1-67 with at least 60%, 70%, 80%, 85%, 90%, or 95% sequence identity to the reference sequence. With respect to the heavy and light chain variable regions, in some embodiments, the CDRs have or include variation relative to reference sequence. In other embodiments, the CDRs do not have or include variation relative to reference sequence. Thus, in some embodiments, the variation is limited to the heavy and light chain variable region framework residues.

Such amino acid substitutions or additions may be introduce naturally occurring (i.e., DNA-encoded) or non-naturally occurring amino acid residues. Such amino acids may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity.,” J. Biol. Chem. 277(30): 26733-26740).

In some embodiments, a humanized antibody is a derivative. Such a humanized antibody can include amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated).

The disclosed antibodies encompass modification of framework residues of the humanized antibodies. Framework residues in the framework regions may be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al. (1988) “Reshaping Human Antibodies For Therapy,” Nature 332:323-327).

A derivative antibody or antibody fragment can be modified by chemical modifications using techniques known to those of skill in the art, including, but not limited to, specific chemical cleavage, acetylation, formulation, metabolic synthesis of tunicamycin, etc. In one embodiment, an antibody derivative possess a similar or identical function as the parental antibody. In another embodiment, an antibody derivative exhibits an altered activity relative to the parental antibody. For example, a derivative antibody (or fragment thereof) can bind to its epitope more tightly or be more resistant to proteolysis than the parental antibody.

Derivatized antibodies can be used to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months.

Substitutions, additions or deletions in the derivatized antibodies may be in the Fc region of the antibody. The Fc portion of an antibody be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed proteins and methods for making them are known in the art, see for example Mueller, J. P. et al. (1997) “Humanized Porcine VCAM-Specific Monoclonal Antibodies With Chimeric Igg2/G4 Constant Regions Block Human Leukocyte Binding To Porcine Endothelial Cells,” Mol. Immun. 34(6):441-452, Swann, P. G. (2008) “Considerations For The Development Of Therapeutic Monoclonal Antibodies,” Curr. Opin. Immun. 20:493-499 (2008), and Presta, L. G. (2008) “Molecular Engineering And Design Of Therapeutic Antibodies,” Curr. Opin. Immun. 20:460-470. In some embodiments the Fe region is the native IgG1, IgG2, or IgG4 Fc region. In some embodiments the Fe region is a hybrid, for example a chimeric consisting of IgG2/IgG4 Fc constant regions. Modifications to the Fc region include, but are not limited to, IgG4 modified to prevent binding to Fe gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region can include the entire hinge region, or less than the entire hinge region.

The antibodies can also be modified by the methods and coupling agents described by Davis et al. (See U.S. Pat. No. 4,179,337). Thus provided are antibodies with a heterologous molecule fused, conjugated, or otherwise attached thereto. Such heterologous molecules may be detectable substance, enzymes, hormones, cell surface receptors, drug moieties, such as: toxins, etc. In some forms, such antibodies are referred to as antibody-drug conjugates (ADCs). In some embodiments, heterologous molecules are polypeptides having at least 10, at least 20, at least 30, at least 40, at least 50, at least 60, at least 70, at least 80, at least 90 or at least 100 amino acids.

Techniques for conjugating such moieties to antibodies are well known; see, e.g., Arnon et al., “Monoclonal Antibodies For Immunotargeting Of Drugs In Cancer Therapy”, in MONOCLONAL ANTIBODIES AND CANCER THERAPY, Reisfeld et al. (eds.), 1985, pp. 243-56, Alan R. Liss, Inc.); Hellstrom et al., “Antibodies For Drug Delivery”, in CONTROLLED DRUG DELIVERY (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in MONOCLONAL ANTIBODIES '84: BIOLOGICAL AND CLINICAL APPLICATIONS, Pinchera et al. (eds.), 1985, pp. 475-506); “Analysis, Results, And Future Prospective Of The Therapeutic Use Of Radiolabeled Antibody In Cancer Therapy”, in MONOCLONAL ANTIBODIES FOR CANCER DETECTION AND THERAPY, Baldwin et al. (eds.), 1985, pp. 303-16, Academic Press; Thorpe et al. (1982) “The Preparation And Cytotoxic Properties Of Antibody-Toxin Conjugates,” Immunol. Rev. 62:119-158; Carter, P. J. et al. (2008) “Antibody-Drug Conjugates for Cancer Therapy,” Cancer J. 14(3):154-169; Alley, S. C. et al. (2010) “Antibody-Drug Conjugates: Targeted Drug Delivery For Cancer,” Curr. Opin. Chem. Biol. 14(4):529-537; Carter, P. et al. (2005) “Designer Antibody-Based Therapeutics For Oncology,” Amer. Assoc. Cancer Res. Educ. Book. 2005(1):147-154; Carter, P. J. et al. (2008) “Antibody-Drug Conjugates For Cancer Therapy,” Cancer J. 14(3):154-169; Chari, R. V. J. (2008) “Targeted Cancer Therapy: Conferring Specificity To Cytotoxic Drugs,” Acc. Chem Res. 41(1):98-107; Doronina, S. O. et al. (2003) “Development Of Potent Monoclonal Antibody Auristatin Conjugates For Cancer Therapy,” Nat. Biotechnol. 21(7):778-784; Ducry, L. et al. (2010) “Antibody-Drug Conjugates: Linking Cytotoxic Payloads To Monoclonal Antibodies,” Bioconjug Chem. 21(1):5-13; Senter, P. D. (2009) “Potent Antibody Drug Conjugates For Cancer Therapy,” Curr. Opin. Chem. Biol. 13(3):235-244; and Teicher, B. A. (2009) “Antibody-Drug Conjugate Targets,” Curr Cancer Drug Targets. 9(8):982-1004.

Also encompassed are antibodies or their antigen-binding fragments that are conjugated to a diagnostic or therapeutic agent or any other molecule. The antibodies can be used diagnostically (in vivo, in situ or in vitro) to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (213Bi), carbon (14C), chromium (51Cr), cobalt (57Co), fluorine (18F), gadolinium (153Gd, 159Gd), gallium (68Ga, 67Ga), germanium (68Ge), holmium (166Ho), indium (115In, 113In, 112In, 111In), iodine (131I, 125I, 123I, 121I), lanthanum (140La), lutetium (177Lu), manganese (54Mn), molybdenum (99Mo), palladium (103Pd), phosphorous (32P), praseodymium (142Pr), promethium (149Pm), rhenium (186Re, 188Re), rhodium (105Rh), ruthenium (97Ru), samarium (153Sm), scandium (47Sc), selenium (75Se), strontium (85Sr), sulfur (35S), technetium (99Tc), thallium (201Ti), tin (113Sn, 117Sn), tritium (3H), xenon (133Xe), ytterbium (169Yb, 175Yb), yttrium (90Y), zinc (65Zn); positron emitting metals using various positron emission tomographies, and nonradioactive paramagnetic metal ions.

The molecules of the disclose can be conjugated to a second antibody to form an antibody heteroconjugate as described by Segal in U.S. Pat. No. 4,676,980. Such heteroconjugate antibodies may additionally bind to haptens (such as fluorescein, etc.), or to cellular markers.

Bispecific and multispecific antibodies that bind the disclosed targets and e.g., a second cancer antigen or immune cell antigen are provided.

Any of the molecules can be fused to marker sequences, such as a peptide, to facilitate purification. In some embodiments, the marker amino acid sequence is a hexa-histidine peptide, the hemagglutinin “HA” tag, which corresponds to an epitope derived from the influenza hemagglutinin protein (Wilson, I. A. et al. (1984) “The Structure Of An Antigenic Determinant In A Protein,” Cell, 37:767-778) and the “flag” tag (Knappik, A. et al. (1994) “An Improved Affinity Tag Based On The FLAG Peptide For The Detection And Purification Of Recombinant Antibody Fragments,” Biotechniques 17(4):754-761).

The molecules of the disclosure can be attached to solid supports, which are particularly useful for immunoassays or purification of the target antigen or of other molecules that are capable of binding to target antigen that has been immobilized to the support via binding to an antibody or antigen-binding fragment of the present invention. Such solid supports include, but are not limited to, glass, cellulose, polyacrylamide, nylon, polystyrene, polyvinyl chloride or polypropylene.

As discussed elsewhere in more detail, also provided are nucleic acid molecules (DNA or RNA) that encode any such antibodies, fusion proteins or fragments, as well as vector molecules (such as plasmids) that are capable of transmitting or of replication such nucleic acid molecules. The nucleic acids can be single-stranded, double-stranded, may contain both single-stranded and double-stranded portions.

### D. Nucleic Acids and Host Cells

Nucleic acids and vectors encoding or expressing the disclosed polypeptides are also described. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence provided herein.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

The nucleic acids can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the polypeptides described herein.

### III. Biomarkers of Aberrantly Expressed CDK5 and Uses Thereof

As introduced above, P-T143 FAM53C, P-T202 LARP6, P-S988 RBL1, P-S17 H1.5, and P-S391 SUV39H1, can be used as biomarkers for MTC diagnosis and/or other diseases and disorders characterized by aberrantly activated Cdk5, identifying the corresponding subject as a target for treatment, and guiding such treatment selection. They can also be used to increase the positive predictive value of current screening modalities and for selection and monitoring the efficacy of treatment regimens. Such compositions and methods are disclosed herein.

The methods typically include detecting one or more of P-T143 FAM53C, P-T202 LARP6, P-S988 RBL1, P-S17 H1.5, and P-S391 SUV39H1 in cells of a sample from a subject. The cells can be suspected of having aberrantly expressed CDK5. Aberrant Cdk5 activity can be characterized by the simultaneous activation of multiple pathways regulating cell proliferation and invasion. In some embodiments, aberrant Cdk5 activity includes over or unregulated expression of Cdk5, hyper-phosphorylation of Cdk5 substrates including, but not limited to, P-T143 FAM53C, P-T202 LARP6, P-S988 RBL1, P-S17 H1.5, and P-S391 SUV39H1, or a combination thereof.

In some embodiments, the cells are suspected or known to be cancer cells. Any of the methods can further include detecting one or more cancer antigen(s) or other disease markers to further characterize the sample. For example, overexpression of Cdk5 co-activators i.e., p35/p25 have been detected in several cancers (colorectal, prostate, pituitary adenoma, pancreatic) that may serve as additional diagnostic markers of aberrant Cdk5 disease. p35 is subjected to rapid proteasomal degradation in its membrane-bound form. Particularly neuroendocrine tumors are characterized by enhanced proteolytic cleavage of p35 to p25. Cdk5 complexed with more stable p25 leads to aberrant kinase activation and subsequent phosphorylation of downstream targets associated with cancer or other diseases

### A. Source of the Biomarkers

The disclosed biomarkers are proteins. Some embodiments provide these biomolecules in isolated form such as in a biological sample. The preferred biological source for detection of the biomarkers is tissue (e.g., tissue suspected of being cancerous) including biopsy material from a tumor, or cells thereof. In some embodiments, the cells are blood cells such as white blood cells such as immune cells, or plasma cells.

In some embodiments, intact cells are subjected to biomarker detection. For example, a sample may be obtained and processed using well-known and routine clinical methods. In some aspects, the biological sample includes a plurality of cells. In certain aspects, the biological sample includes fresh or frozen tissue. In specific aspects, the biological sample includes formalin fixed, paraffin embedded tissue.

In some embodiments, the cells are permeabilized. In some embodiments, a cell lysate or homogenate is subjected to biomarker detection.

### B. Methods of Detecting Biomarkers

The disclosed biomarkers for aberrant expression of Cdk5 are typically detected using one or more of the antibodies or other antigen binding molecules provided herein. The biomarkers can be detected by any suitable method utilizing the provided antibodies. In preferred embodiments, the biomarkers are detected and/or measured by an immunoassay. Immunoassays utilize biospecific capture reagents, such as antibodies, to capture or locate the biomarkers.

The steps of various useful immunodetection methods have been described in the scientific literature. In general, the immunobinding methods include obtaining a sample, and contacting the sample with an antibody specific for the protein to be detected, as the case may be, under conditions effective to allow the formation of immunocomplexes. In general, the detection of immunocomplex formation is well known in the art and may be achieved through the application of numerous approaches. These methods are generally based upon the detection of a label or marker, such as any of those radioactive, fluorescent, biological and enzymatic tags. Of course, one may find additional advantages through the use of a secondary binding ligand such as a second antibody and/or a biotin/avidin ligand binding arrangement, as is known in the art.

The antibody employed in the detection may itself be linked to a detectable label (also referred to as a detectable substance or reporter), wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Traditional immunoassays including, for example, sandwich immunoassays including ELISA or fluorescence-based immunoassays, as well as other enzyme immunoassays can be used for detecting the biomarkers. In other embodiments, the detection of the biomarker is carried out on slides of test material (e.g., immunohistochemistry), Western blotting, surface plasmon resonance (e.g. Biacore), or by flow cytometry (FACS) analysis), e.g., as exemplified in the experiments below.

Other specific examples include, but are not limited to, enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, and immuno-chromatography assays. Most assays now use nonradioactive labels. Enzyme immunoassays (enzyme-linked immunosorbent assays, or ELISA; immunometric assays) can use enzymes as labels, such as, for example, horseradish peroxidase or alkaline phosphatase. Chemiluminescent immunoassays (CIA) can use luminol. Fluorimetric immunoassays (FIA) use fluorescent compounds (e.g., fluorescein) as labels.

The assays can be homogenous or heterogeneous assays, competitive and non-competitive assays. There are four main kinds of ELISA: sandwich, competitive, direct, and indirect assays. These methods differ in how the antibody or antigen is attached to the solid plate, and how the signal is detected. For example, in a sandwich ELISA, for example, an antibody is immobilized on a plate. The sample containing the target antigen is added, which binds to the antibody and so is immobilized on the plate. Next, a second type of antibody is added, which also binds to the target antigen on the plate, forming a ‘sandwich’ with the target antigen in the middle. The second antibody is linked to an enzyme, called a reporter enzyme, which allows the binding reaction to be measured by creating a color signal. To create this signal, first any unbound antibody is washed away, and a colorimetric substrate is added. The enzyme catalyzes a reaction of the substrate, creating a color change. A stronger color signal indicates more target antigen is present. An example of this is a home pregnancy test. In some embodiments, the first or second antibody is one of the disclosed antibodies, and the other antibody is one that detects the protein of interest or the biomarker (e.g., FAM53C, LARP6, RBL1, H1.5, and SUV39H1), but may or may not detect its phosphorylated state, and thus may target a different antigen of the protein (e.g., a non-phosphorylated antigen).

In some embodiments, the assay is in the form of a sandwich assay, which is a noncompetitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person.

Immunohistochemistry (IHC) is a process of localizing antigens (e.g., proteins) in tissue utilizing antigen-specific antibodies. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avidin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag.

Quantitative immunochemical techniques can also be used.

For example, the Quantitative Tissue Biomarker Platform from HistoRx and/or measuring immunofluorescence level(s) can be used to quantify levels of biomarkers.

Western blotting can be used and can be quantitative or qualitative. A typical Western blotting procedure includes the steps of immunoprecipitating a target protein from a lysate of cells expressing the protein, performing an SDS-PAGE with said protein, transferring the protein to a nitrocellulose membrane, incubating the nitrocellulose membrane with said antibody, detecting said antibody with a secondary antibody conjugated to a fluorescent or chromogenic compound (e.g. peroxidases such as horseradish peroxidase (HRP), alkaline phosphatase (AP), IRDye near-infrared (NIR) fluorescent dyes), and quantifying the respective signal of said compound (e.g. fluorescence, luminescence, chromogenic enzyme substrate).

The ratio of two signals generated by Western blotting employing the same antibody but two different samples can be calculated, thereby determining how much more/less (fold-change) of the biomarker is present in one sample compared to another.

In some embodiments, the methods provided herein involve determining the presence, absence, and/or concentration of biomarker in a cell, and/or the number of biomarker positive cells in sample by fluorescence activated cell sorting (FACS) using a flow cytometry device (e.g., Beckman Coulter Z2 Coulter Counter, Beckman Coulter Inc.). In some embodiments, a FACs-based method include the step of preparing the output composition for detection by flow cytometry before the biomarker can be detected. For example, the output composition can be incubated with a fluorescently labeled antibody that is specific for one or more biomarkers, and then the sample can be analyzed using a flow cytometer. In some embodiments, the cells are permeabilized to facilitate antibody access to intracellular biomarker. In flow cytometry, cells bound by fluorescently labeled affinity reagents are carried in a fluidic stream, are separated based on size and/or fluorescent signal and are subsequently analyzed and counted using a FACS software program (e.g., FlowJo software). The number or approximate number of cells can be determined by detection of the fluorescent signal, which optionally can be determined or processed by the FACS software program to provide the total or approximate number of particles in the output composition.

In some embodiments, a sample is analyzed by means of a biochip. Biochips generally include solid substrates and have a generally planar surface, to which a capture reagent (also called an adsorbent or affinity reagent) is attached. Frequently, the surface of a biochip includes a plurality of addressable locations, each of which has the capture reagent bound there.

Protein biochips are biochips adapted for the capture of polypeptides. Many protein biochips are described in the art. These include; for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden).

Photonic biosensors can be used in label-free assays. For example, photonic biosensors combine photonic sensing with bio recognition technology to create label-free testing on-chip. Instead of moving electrons around on silicon chips, light is moved around on silicon chips via waveguides. This technology has allowed the development of miniature lab-on-a-chip label-free immunoassay (LFIA) devices. These devices are functionalized with capture antibodies and have a resonance condition of light. This resonance wavelength will be shifted by a reaction between the capture antibody and the target antigen due to the change in refractive index. Measuring the shift in resonance wavelength provides a readout of a binding event. Label-free assays therefore enable the detection of antigen-antibody binding without the use of an additional label, resulting in increased assay sensitivity and decreased working time.

Any of the detection methodologies can be multiplexed to detect two, three, four, or all five of the disclosed biomarker. In a particular embodiments, the methodology is or includes a multiplex immunoassay utilizing Luminex microbead technology.

### C. Diagnosis

### 1. Single Markers

The biomarkers can be used in diagnostic tests to assess cancer and/or other Cdk5-related disease and disorder status in a subject, e.g., to distinguish between normal cells and diseased cells, and disease status. For example, disease status includes, without limitation, the presence or absence of disease (e.g., cancer v. non-cancer), characterization of cells including cancer cells (e.g., level of aberrant Cdk5 activity), the risk of developing disease, the stage of the disease (e.g., non-invasive or early-stage cancer v. invasive or metastatic cancer), the progress of disease (e.g., progress of disease or remission of disease over time) and the effectiveness or response to treatment of disease. Based on this status, further procedures may be indicated, including additional diagnostic tests or therapeutic procedures or regimens. Representative cancers and therapies are discussed in more detail below.

The biomarkers discussed herein can be present and/or expressed in cancer including but not limited to MTC, and, therefore, each is individually useful in aiding in the determination of cancer and/or other Cdk5-related diseases and disorders. The method involves, first, measuring the selected biomarker in a subject sample using the methods described herein, and, second, comparing the measurement with a diagnostic amount or cut-off that distinguishes a positive cancer and/or other Cdk5-related disease and disorder status from a negative cancer and/or other Cdk5-related disease and disorder status. The diagnostic amount represents a measured amount of a biomarker above which a subject is classified as having a particular status. For example, because the biomarker is up-regulated compared to normal during cancer and/or other Cdk5-related diseases and disorders, then a measured amount above the diagnostic cutoff provides a diagnosis or status of the cancer and/or other Cdk5-related diseases and disorders. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different cancer statuses and drawing the cut-off to suit the diagnostician's desired levels of specificity and sensitivity.

### 2. Combinations of Markers

While individual biomarkers are useful diagnostic biomarkers, a combination of biomarkers may provide greater predictive value of a particular status than single biomarkers alone. Specifically, the detection of a plurality of biomarkers in a sample can increase the sensitivity and/or specificity of the test. Thus, in one embodiment, two or more, three or more, four or more or even all five of the biomarkers can be detected and used to assess the status of cancer and/or other Cdk5-related disease and disorder in a subject.

### D. Determining Risk of Developing Disease

Methods for determining the risk of developing disease in a subject are also provided. Biomarker amounts or patterns can be characteristic of various risk states, e.g., high, medium, or low. The risk of developing a disease is determined by measuring the relevant biomarker or biomarkers and then either submitting them to a classification algorithm or comparing them with a reference amount and/or pattern of biomarkers that is associated with the particular risk level.

### E. Determining Stage of Disease

Another embodiment provides methods for determining the stage of disease in a subject. Each stage of the disease can have a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

### F. Determining Course (Progression/Remission) of Disease

Still another embodiment provides methods for determining the course of disease in a subject. Disease course refers to changes in disease status over time, including disease progression (worsening) and disease regression (improvement). Over time, the amounts or relative amounts (e.g., the pattern) of the biomarkers changes. This method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

### G. Subject Management

In certain embodiments of the method including the detection and/or analysis of one or more biomarkers further include managing subject treatment based on the status. Such management includes the actions of the physician or clinician subsequent to determining cancer and/or other Cdk5-related disease and disorder status. For example, if a physician makes a diagnosis of cancer and/or other Cdk5-related disease and disorder, then a certain regime of treatment, such as prescription or administration of chemotherapy, radiation, immunotherapy, including, but not limited to administration of the compositions discussed in more detail below, might follow. Alternatively, a diagnosis of non-cancer or benign tumor might be followed with further testing to determine a specific disease that the patient might be suffering from. Also, if the diagnostic test gives an inconclusive result on cancer and/or other Cdk5-related disease and disorder, further tests may be required.

One embodiment provides a method for selecting a subject for treatment for cancer and/or other Cdk5-related disease and disorder by detecting the presence or quantity of one or more biomarkers provided herein in a sample from a subject suspected of having cancer and/or other Cdk5-related disease and disorder, comparing the levels of biomarker in the sample to a predetermined standard, wherein the patient is selected for treatment for cancer and/or other Cdk5-related disease and disorder if certain biomarkers or levels of biomarkers are detected in the sample. Such treatments can be those known to be effective and/or preferred for treating subjects with aberrant Cdk5-positive conditions.

In some embodiments, the methods additionally or alternatively include identifying the subject as not having a Cdk5-related disease and disorder, when the test is negative. Thus, although the subject may have a cancer or another diseases or disorder, the subject can be identified as negative for aberrant Cdk5-related cancer and other diseases and disorder. Such embodiments may lead to selection of alternative treatments and may avoid treatments known to be effective or preferred for treating subjects with aberrant Cdk5-positive conditions, and/or may include treatments that are known not to be effective and/or preferred for treating subjects with aberrant Cdk5-positive conditions.

Additional embodiments relate to the communication of assay results or diagnoses or both to technicians, physicians or patients, for example. In certain embodiments, computers will be used to communicate assay results or diagnoses or both to interested parties, e.g.: physicians and their patients. In some embodiments, the assays will be performed or the assay results analyzed in a country or jurisdiction which differs from the country or jurisdiction to which the results or diagnoses are communicated.

In a preferred embodiment a diagnosis based on the presence or absence in a test subject of any of the disclosed biomarkers is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. In certain embodiments all or some of the method steps, including the assaying of samples, diagnosing of diseases, and communicating of assay results or diagnoses, may be carried out in diverse (e.g., foreign) jurisdictions.

### H. Biomarkers in Screening Assays

The biomarkers can be used to screen for compounds that modulate the expression of the biomarkers in vitro or in vivo, which compounds in turn may be useful in treating or preventing cancer and/or other Cdk5-related disease and disorder in patients. Compounds suitable for therapeutic testing may be screened initially by identifying compounds which reduce the presence of one or more biomarkers in the cancer and/or other Cdk5-related disease and disordered cells.

Test compounds capable of modulating the presence and/or expression of any of the biomarkers in cancer cells or other Cdk5-related disease and disorder may be administered to patients who are suffering from or are at risk of developing cancer having the biomarkers. For example, the administration of a test compound that decreases the activity of a particular biomarker may decrease the risk of cancer and/or other Cdk5-related disease and disorder in a patient if the increased activity of the biomarker is responsible or indicative, at least in part, for the onset of the cancer and/or other Cdk5-related disease and disorder.

At the clinical level, screening a test compound includes obtaining samples from test subjects before and after the subjects have been exposed to a test compound. The levels in the samples of one or more of the biomarkers can be measured and analyzed to determine whether the levels of the biomarkers change after exposure to a test compound. The samples can be analyzed by any appropriate means known to one of skill in the art including e.g., by the means described herein. In a further embodiment, the changes in the level of expression of one or more of the biomarkers can be measured using in vitro methods and materials. For example, human tissue cultured cells which express, or are capable of expressing, one or more of the biomarkers may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with cancer and/or other Cdk5-related disease and disorder, test compounds will be screened for their ability to slow or stop the progression of the disease.

I. Assessing the Effectiveness of Treatment or Risk for Developing Cancer and/or Other Cdk5-Related Disease and Disorder

Methods for determining the course of cancer and/or other Cdk5-related diseases and disorders in a subject are also provided. Disease course refers to changes in disease status over time, including disease progression (worsening) and disease regression (improvement). Over time, the amounts or relative amounts (e.g., the pattern) of the biomarkers changes. Accordingly, this method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

In yet another example, the biomarkers can be used in studies to determine if the subject is at risk for developing cancer and/or other Cdk5-related disease and disorder.

### IV. Methods of Treatment

Any of the disclosed methods can be coupled to a method of treating a subject in need thereof. Thus, any of the disclosed methods can further include treating a positive for cancer and/or other Cdk5-related disease and disorder with a treatment known to be effective and/or preferred for treating subjects with aberrant Cdk5-positive conditions.

In other embodiments, the method includes treating a subject with a disease or disorder, but negative for aberrant Cdk5 activity with a treatment not to be effective and/or preferred for treating subjects with aberrant Cdk5-positive conditions and/or selecting against treatments known to be effective or preferred for treating subjects with aberrant Cdk5-positive conditions.

In certain embodiments, the compositions are administered systemically, locally, or regionally. In some embodiments, the compositions are taken orally, injected, topically applied, or otherwise administered directly into the vasculature or onto vascular tissue at or adjacent to a site of cancerous growth. Typically, local administration causes an increased localized concentration of the compositions, which is greater than that which can be achieved by systemic administration.

### A. Conditions to be Diagnosed and Treated

The subject to be treated can have a disease, disorder, or condition such as but not limited to, cancer, neurological diseases (such as Alzheimer's disease, Parkinson's disease, Huntington's disease, Cerebral ischemia, Traumatic brain injury, and addiction), an immune system disorder such as autoimmune disease, an inflammatory disease, an infectious disease, or combinations thereof. In some embodiments, the disease, disorder, or condition is characterized by aberrant (e.g., increase) Cdk5 activity. The disease, disorder, or condition can be associated with an elevated expression or specific expression of an antigen, e.g., P-T143 FAM53C, P-T202 LARP6, P-S988 RBL1, P-S17 H1.5, and/or P-S391 SUV39H1.

Typically, the cancer or other disease or disorder is analyzed for one or more of P-T143 FAM53C, P-T202 LARP6, P-S988 RBL1, P-S17 H1.5, and P-S391 SUV39H1, increased presence of which indicates the subject has a disease or disorder associated aberrant Cdk5 activity.

In some embodiments, the subject is previously, contemporaneously, or subsequently analyzed for one or more additional indicators for cancer. Such indicators can be, for example, morphological or molecular (e.g., other biomarkers). For example, in some embodiments, the subject is analyzed for one or more cancer antigens were detected on the cancer cells, optionally leading to a cancer diagnosis. Thus, in some embodiments, the cancer was already diagnosed and/or characterized prior to or as an adjunct to treatment.

Typically, the subjects to be treated have a proliferative disease, such as a benign or malignant tumor. In some embodiments, the subjects to be treated have been diagnosed with stage I, stage II, stage III, or stage IV cancer. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

In some embodiments, the compositions and methods are useful for treating subjects having benign or malignant tumors by delaying or inhibiting the growth of a tumor in a subject, reducing the growth or size of the tumor, inhibiting, or reducing metastasis of the tumor, and/or inhibiting or reducing symptoms associated with tumor development or growth.

Malignant tumors that may be treated are classified according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Tumors may develop in various organs or tissues throughout the body to establish malignant cancer.

The types of cancer that can be treated with the provided compositions and methods include, but are not limited to, cancers such as colorectal cancer, peritoneal carcinomatosis, pancreatic cancer, multiple myeloma, sarcomas, brain, breast, esophageal, liver, lung, stomach, and uterine. In some embodiments, the compositions are used to treat multiple cancer types concurrently. The compositions can also be used to treat metastases or tumors at multiple locations.

Typically, the cancer is one in which Cdk5 is up-regulated. Cdk5 has emerged as an important instigator of tumorigenic signaling in several types of neuroendocrine tumors (NETs) including pancreatic, adrenal, thyroid, and gastrointestinal tumors. Non-neuroendocrine malignancies such as colorectal cancer and melanomas are also afflicted with pathological activation of Cdk5 signaling. Cdk5 has been shown to promote tumor growth and invasion by regulating the activity of proteins involved in metabolic reprogramming, cytoskeletal remodeling, and epithelial-mesenchymal transition.

Cdk5 is reported to be over-expressed or hyperactivated in various cancer tissues and tumor cell lines. For example, in patients with lung cancer it is associated with clinical pathological characteristics and poorer prognoses. In head and neck squamous cell carcinoma, aberrant over-expression of Cdk5 significantly induces tumor cell motility and epithelial mesenchymal transition (EMT), which is considered as a pivotal process of cancer metastasis. In hepatocellular carcinoma (HCC), over-expression and hyperactivation of Cdk5 play an oncogenic role by inducing proliferation and clonogenic growth of HCC. In pancreatic cancer cells, Cdk5 is widely active. In breast cancer cells treated with transforming growth factor beta 1 (TGF-β1, an EMT inducer), both p35 and Cdk5 are up-regulated and hyperactivated, leading to subsequent EMT and cancer cell motility by phosphorylating Fak, which is known to be involved in cellular adhesion and spreading processes. Cdk5 and p35 are also significantly increased in clinical breast cancer tissues and in breast cancer cell lines exposed to paclitaxel at transcriptional and translational levels. See, e.g., Liu, et al., Molecular Cancer volume 16, Article number: 60 (2017), 9 pages, and references cited therein.

Furthermore, results provided in the experiments below not only illustrate the importance of aberrant Cdk5 expression in Medullary Thyroid Carcinoma (MTC), but also highlight its involvement in metabolic dysregulations instilling aggressive MTC phenotype. An increasing number of studies show that malignant tumors are highly dependent on lipid metabolism and fatty acid synthesis for growth and survival [58, 59]. Cdk5 is an emerging candidate entangled in several metabolic conditions including cancer, diabetes, and obesity [60] [61]. Cdk5-dependent phosphorylation of PPARγ and PRKAG2 impairs key metabolic sensors such as adipsin, adiponectin, and AMPK kinase [12, 62]. A direct connection between Cdk5 and lipid metabolism was recently reported where Cdk5-mediated phosphorylation of acetyl-CoA synthetase 2 (ACSS2) was shown to promote glioblastoma growth by regulating lipid production [63]. Thus, in some embodiments, the subject has a non-cancer metabolic disorder such as diabetes or obesity.

Therapeutic treatment can involve administering to a subject a therapeutically effective amount of a treatment after cancer or another condition has been diagnosed.

### B. Therapies

In some embodiments, patients are also subject to one or more therapies or procedures for the treatment of the disease or disorder. When two or more therapies or procedures are used, they can be simultaneous or sequential combination therapy.

In some embodiments, the therapy is a conventional treatment for cancer, more preferably a conventional treatment for the particular cancer type, e.g., prostate or breast cancer. For example, in some embodiments, the additional therapy or procedure is surgery, a radiation therapy, or chemotherapy.

In some embodiments, the conventional cancer therapy is in the form of one or more active agents. Therefore, in some embodiments, the methods administer compositions in combination with one or more additional active agents. Such active agent can be, for example, chemotherapeutic agents, cytokines, chemokines, radiation therapy, or immunotherapy. The majority of chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, the therapy is a chemotherapeutic agent. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5, and combinations thereof.

In some embodiments, the treatment is or includes immunotherapy such as inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some embodiments, the treatment is or includes adoptive T cell therapy. Methods of adoptive T cell therapy are known in the art and used in clinical practice. Generally adoptive T cell therapy involves the isolation and ex vivo expansion of tumor-specific T cells to achieve greater number of anti-tumor T cells than what could be obtained by vaccination alone. The tumor-specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

In some embodiments, the treatment is or includes a cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen is delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance the body's own anti-tumor immune functions.

In preferred embodiments, for subjects with aberrant Cdk5-positive, the treatment targets Cdk5. Dinaciclib (formerly SCH727965), a potent and selective small molecule inhibitor of Cdk2, Cdk5, Cdk1 and Cdk9, is the first Cdks inhibitor to enter the clinic trail. Its promising anti-cancer results and acceptable safety profile have been respectively proven in preclinical studies and human phase I trial. In preclinical model of ovarian cancer, SCH727965 has been shown to synergize with cisplatin in killing cancer cells. In phase I trial of SCH727965, subjects with advanced malignancies experienced acceptable and tolerable dose-limiting toxicities, including orthostatic hypotension, elevated uric acid, nausea, anemia, neutropenia, etc. Because Cdk5 is hyperactivated in a degradation-dependent pathway by proteasome calpain, it is believed that the proteasome inhibitors such as Bortezomib (Velcade), which has been approved by FDA for mantle cell lymphoma treatment, may play anti-cancer function to some extent by regulating Cdk5-related pathways in other cancers. Liu, et al., Molecular Cancer volume 16, Article number: 60 (2017), 9 pages, and references cited therein.

In some embodiments, the treatment is a functional nucleic acid that targets Cdk5. Functional nucleic acid molecules can be divided into the following non-limiting categories: antisense molecules, siRNA, miRNA, aptamers, ribozymes, triplex forming molecules, RNAi, external guide sequences, and other gene editing compositions. Such compositions can be used to reduce expression of Cdk5.

Cdk5 also plays an important role in treatment of chemo-resistant cancers. For example, in cervical cancer the expression of cyclin I is up-regulated by cisplatin treatment, which in turn confers cancer cells resistance to cisplatin by activating Cdk5 and its anti-apoptosis effect. Knockdown of Cdk5 with siRNA can significantly increase the sensitivity to cisplatin in Hela cell lines with over-expressed cyclin I [76]. Similar results are also observed in Cdk5-inhibited HCC cells [66] and Cdk5-depleted ovarian cancer cell lines [77], in which cancer cells exhibit higher sensitivity to DNA damaging agents. Thus, some treatments include simultaneous reduction of Cdk5 activity, and chemotherapy.

In some embodiments, the treatment is or includes roscovitine. Roscovitine [CY-202, (R)-Roscovitine, Seliciclib] is a small molecule that inhibits cyclin-dependent kinases (CDKs) through direct competition at the ATP-binding site (Cicenas, et al., Ann Transl Med. 2015 June; 3(10):135. doi: 10.3978/j.issn.2305-5839.2015.03.61. PMID: 26207228; PMCID: PMC4486920.). It is a broad-range purine inhibitor, which inhibits CDK1, CDK2, CDK5 and CDK7, but is a poor inhibitor for CDK4 and CDK6. Roscovitine is widely used as a biological tool in cell cycle, cancer, apoptosis and neurobiology studies. Moreover, it is currently evaluated as a potential drug to treat cancers, neurodegenerative diseases, inflammation, viral infections, polycystic kidney disease and glomerulonephritis.

In some embodiments, the treatment is or includes Indolinone A. Indolinone A. Selective Cdk5 inhibition by Indolinone A (IndoA) blocked all human NE cancer cell growth more potently than it affected normal human fibroblasts or rat INS cells (Carter, et al., PNAS, 117 (31) 18401-18411 (2020) doi:10.1073/pnas.2010103117.

In some embodiments, the treatment is or includes MRT3-007. MRT3-007 is a potent Cdk5 inhibitor that can reverse the phospho-cascade, invoking a senescence-like phenotype, a therapeutic approach that halted tumor progression in vivo (Gupta, et al., Cell Rep. 2022 Aug. 16; 40(7):111218. doi: 10.1016/j.celrep.2022.111218. PMID: 35977518; PMCID: PMC9822535).

In some embodiments, the treatment is or includes the peptide inhibitor TFP5/TP5. TP5 has been shown to decrease glioblastoma cell viability and tumor growth by blocking cell cycle and increasing apoptosis through the inhibition of ATM phosphorylation, and act synergistically with radiotherapy and temozolomide by impairing DNA damage repair (Tabouret, et al., Cancers (Basel). 2020 Jul. 17; 12(7):1935. doi: 10.3390/cancers12071935. PMID: 32708903; PMCID: PMC7409269.).

### C. Controls

The effect of the compositions can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout, including but not limited to, the presence of level of the biomarkers provided herein. For example, the effect of a treatment on a particular cancer can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art. In some embodiments, an untreated control subject suffers from the same disease or condition as the treated subject.

### V. Kits

The disclosed antibodies and optionally additional elements for carrying out the disclosed methods can be packaged in kit. In some embodiments, the kit can includes the materials shipping and storage and/or administration, and instructions for using the materials. Instructions can direct the user on how to apply the disclosed antibodies in detection and/or diagnostic assay, and/or how to select a tested subject for treatment and/or how to the select the treatment. In other embodiments, the kit can include a single dose or a plurality of doses of a treatment in a pharmaceutically acceptable carrier for shipping and storage and/or administration, and instructions for administering the compositions. Specifically, the instructions direct that an effective amount of the composition be administered to an individual with a particular condition/disease as indicated. The composition can be formulated as described above with reference to a particular treatment method and can be packaged in any convenient manner.

The present invention can be further understood by reference to the following numbered paragraphs:

1. An antibody or other molecule including an antigen binding domain of an antibody that immunospecifically binds to the epitope:

(i)
(SEQ ID NO: 1)
CSIYI-pS-PHKN;
(ii)
(SEQ ID NO: 14)
CAPVEK-pS-PAK;
(iii)
(SEQ ID NO: 27)
CALA-pT-PQKNGRV;
(iv)
(SEQ ID NO: 40)
CLAGLPG-pS-PKKRVR;
or
(v)
(SEQ ID NO: 53)
CAPSKLW-pT-PIKH.

2. The antibody or other molecule of paragraph 1, wherein the antigen binding domain includes six complementarity-determining regions (CDRs), wherein the CDRs include one, two, three, four, five, or six consensus CDRs of the CDRs of:
(i) anti-RBL1 antibody RBL1-1;
(ii) anti-H1.5 antibody H1-5-1;
(iii) anti-LARP6 antibody LARP6-1;
(iv) anti-SUV39H1 antibody SUV39H1-1; or
(v) anti-FAM53C antibody FAM53C-1.

3. The antibody or other molecule of paragraph 2, wherein the CDRs include one, two, three, four, five, or six consensus CDRs of the CDRs of:
(i) a heavy chain variable region including the amino acid sequence of SEQ ID NO:2 and/or a light chain variable region including the amino acid sequence of SEQ ID NO:3;
(ii) a heavy chain variable region including the amino acid sequence of SEQ ID NO:15 and/or a light chain variable region including the amino acid sequence of SEQ ID NO:16;
(iii) a heavy chain variable region including the amino acid sequence of SEQ ID NO:28 and/or a light chain variable region including the amino acid sequence of SEQ ID NO:29;
(iv) a heavy chain variable region including the amino acid sequence of SEQ ID NO:41 and/or a light chain variable region including the amino acid sequence of SEQ ID NO:42; or
(v) a heavy chain variable region including the amino acid sequence of SEQ ID NO:54 and/or a light chain variable region including the amino acid sequence of SEQ ID NO:55.

4. The antibody or other molecule of paragraphs 2 or 3, wherein the CDRs include one, two, three, four, five, or six including the amino acid sequence(s) of:
(i) SEQ ID NOS:4-9 or a variant thereof with at least 70% sequence identity thereto;
(ii) SEQ ID NOS:17-22 or a variant thereof with at least 70% sequence identity thereto;
(iii) SEQ ID NOS:30-35 or a variant thereof with at least 70% sequence identity thereto;
(iv) SEQ ID NOS:41-48 or a variant thereof with at least 70% sequence identity thereto; or
(v) SEQ ID NOS:56-59, 34, and 60 or a variant thereof with at least 70% sequence identity thereto.

5. The antibody or other molecule of any one of paragraphs 2-4, wherein the CDRs include the six CDRs of:
(i) SEQ ID NOS:4-9 in the same order and orientation as presented in SEQ ID NOS:2 and 3, respectively;
(ii) SEQ ID NOS: 17-22 in the same order and orientation as presented in SEQ ID NOS:15 and 16, respectively;
(iii) SEQ ID NOS:30-35 in the same order and orientation as presented in SEQ ID NOS:28 and 29, respectively;
(iv) SEQ ID NOS:41-48 in the same order and orientation as presented in SEQ ID NOS:41 and 42, respectively; or
(v) SEQ ID NOS:56-59, 34, and 60 in the same order and orientation as presented in SEQ ID NOS:54 and 55, respectively.

6. The antibody or other molecule of any one of paragraphs 1-5, including
(i) a heavy chain variable region including the amino acid sequence of SEQ ID NOS:2 or a variant thereof with at least 70% sequence identity thereto and/or a light chain variable region including the amino acid sequence of SEQ ID NOS:3 or a variant thereof with at least 70% sequence identity thereto;
(ii) a heavy chain variable region including the amino acid sequence of SEQ ID NOS:15 or a variant thereof with at least 70% sequence identity thereto and/or a light chain variable region including the amino acid sequence of SEQ ID NOS:16 or a variant thereof with at least 70% sequence identity thereto;
(iii) a heavy chain variable region including the amino acid sequence of SEQ ID NOS:28 or a variant thereof with at least 70% sequence identity thereto and/or a light chain variable region including the amino acid sequence of SEQ ID NOS:29 or a variant thereof with at least 70% sequence identity thereto;
(iv) a heavy chain variable region including the amino acid sequence of SEQ ID NOS:41 or a variant thereof with at least 70% sequence identity thereto and/or a light chain variable region including the amino acid sequence of SEQ ID NOS:42 or a variant thereof with at least 70% sequence identity thereto; or
(v) a heavy chain variable region including the amino acid sequence of SEQ ID NOS:54 or a variant thereof with at least 70% sequence identity thereto and/or a light chain variable region including the amino acid sequence of SEQ ID NOS:55 or a variant thereof with at least 70% sequence identity thereto.

7. The antibody or other molecule of any one of paragraphs 1-6 including
(i) a heavy chain variable region wherein the heavy chain variable region includes the amino acid sequence of SEQ ID NOS:2 and a light chain variable region wherein the light chain variable region includes the amino acid sequence of SEQ ID NOS:3 or a variant thereof with at least 70% sequence identity thereto;
(ii) a heavy chain variable region wherein the heavy chain variable region includes the amino acid sequence of SEQ ID NOS:15 and a light chain variable region wherein the light chain variable region includes the amino acid sequence of SEQ ID NOS:16 or a variant thereof with at least 70% sequence identity thereto;
(iii) a heavy chain variable region wherein the heavy chain variable region includes the amino acid sequence of SEQ ID NOS:28 and a light chain variable region wherein the light chain variable region includes the amino acid sequence of SEQ ID NOS:29 or a variant thereof with at least 70% sequence identity thereto;
(iv) a heavy chain variable region wherein the heavy chain variable region includes the amino acid sequence of SEQ ID NOS:41 and a light chain variable region wherein the light chain variable region includes the amino acid sequence of SEQ ID NOS:42 or a variant thereof with at least 70% sequence identity thereto; or
(v) a heavy chain variable region wherein the heavy chain variable region includes the amino acid sequence of SEQ ID NOS:54 and a light chain variable region wherein the light chain variable region includes the amino acid sequence of SEQ ID NOS:55 or a variant thereof with at least 70% sequence identity thereto.

8. The antibody or other molecule of any one of paragraphs 1-6 including
(i) a heavy chain variable region wherein the heavy chain variable region includes the amino acid sequence of SEQ ID NOS:2 and a light chain variable region wherein the light chain variable region includes the amino acid sequence of SEQ ID NOS:3 or a variant thereof with at least 70% sequence identity thereto, without any variation in the CDRs of SEQ ID NOS:2 and 3;
(ii) a heavy chain variable region wherein the heavy chain variable region includes the amino acid sequence of SEQ ID NOS:15 and a light chain variable region wherein the light chain variable region includes the amino acid sequence of SEQ ID NOS:16 or a variant thereof with at least 70% sequence identity thereto, without any variation in the CDRs of SEQ ID NOS:15 and 16;
(iii) a heavy chain variable region wherein the heavy chain variable region includes the amino acid sequence of SEQ ID NOS:28 and a light chain variable region wherein the light chain variable region includes the amino acid sequence of SEQ ID NOS:29 or a variant thereof with at least 70% sequence identity thereto, without any variation in the CDRs of SEQ ID NOS:28 and 29;
(iv) a heavy chain variable region wherein the heavy chain variable region includes the amino acid sequence of SEQ ID NOS:41 and a light chain variable region wherein the light chain variable region includes the amino acid sequence of SEQ ID NOS:42 or a variant thereof with at least 70% sequence identity thereto, without any variation in the CDRs of SEQ ID NOS:41 and 42; or
(v) a heavy chain variable region wherein the heavy chain variable region includes the amino acid sequence of SEQ ID NOS:54 and a light chain variable region wherein the light chain variable region includes the amino acid sequence of SEQ ID NOS:55 or a variant thereof with at least 70% sequence identity thereto, without any variation in the CDRs of SEQ ID NOS:54 and 55;

9. The antibody or other molecule of any one of paragraphs 1-8 including:
(i) the amino acid sequences of SEQ ID NOS:10 and/or 11, with or without the cleavable signal sequence;
(ii) the amino acid sequences of SEQ ID NOS:23 and/or 24, with or without the cleavable signal sequence;
(iii) the amino acid sequences of SEQ ID NOS:36 and/or 37, with or without the cleavable signal sequence;
(iv) the amino acid sequences of SEQ ID NOS:49 and/or 50, with or without the cleavable signal sequence; or
(v) the amino acid sequences of SEQ ID NOS:61 and/or 62, with or without the cleavable signal sequence.

10. The antibody or other molecule of any one of paragraphs 1-9 includes an amino acid sequence encoded by the nucleic acid sequence of:
(i) SEQ ID NOS:12 and/or 13;
(ii) SEQ ID NOS:25 and/or 26;
(iii) SEQ ID NOS:38 and/or 39;
(iv) SEQ ID NOS:51 and/or 52; or
(v) SEQ ID NOS:63 and/or 64.

11. The antibody or molecule of any one of paragraphs 1-10 including a heavy chain constant region.

12. The antibody or molecule of paragraph 11, wherein the heavy chain constant region includes the amino acid sequence of SEQ ID NO:65 or a variant thereof with at least 70% sequence identity to SEQ ID NO:65.

13. The antibody or molecule of any one of paragraphs 1-12 including a light chain constant region.

14. The antibody or molecule of paragraph 13, wherein the light chain constant region includes the amino acid sequence of SEQ ID NO:66 or 67 or a variant thereof with at least 70% sequence identity to SEQ ID NO:66 or 67.

15. The antibody or molecule of any one of paragraphs 1-14, wherein the molecule or antibody is an antibody.

16. The antibody or molecule of paragraph 15, wherein the antibody is an intact antibody or functional antibody fragment or fusion protein.

17. The antibody or molecule of paragraph 16, wherein the functional fragment or fusion protein is selected from Fab fragments, F(ab′)2 fragments, Fab′ fragments, Fv fragments, recombinant IgG (rIgG) fragments, single chain antibody fragments optionally single chain variable fragments (scFv), and single domain antibodies optionally selected from sdAb, sdFv, and nanobody fragments.

18. The antibody or molecule of any one of paragraphs 15-17, wherein the antibody is selected from intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv.

19. The antibody or molecule of any one of paragraphs 11-14 wherein the antibody is an IgM, IgE, IgA, IgD, or IgG optionally an IgG1, IgG2, IgG3, or IgG4.

20. The antibody or molecule of any one of paragraphs 1-19, wherein the molecule or antibody is detectably labeled or includes a conjugated toxin, drug, receptor, enzyme, receptor ligand.

21. A method of detecting Cdk5 activity including detecting the level of phosphorylation of one or more of P-T143 FAM53C, P-T202 LARP6, P-S988 RBL1, P-S17 H1.5, and P-S391 SUV39H1 by contacting a biological sample with one or more antibodies of any one of paragraphs 1-20, and detecting binding between the antibody or antibodies and the one or more of FAM53C, LARP6, RBL1, H1.5, and SUV39H1.

22. The method of paragraph 21 further including determining that the sample includes aberrant Cdk5 activity if the level of detected binding is higher in the biological sample than in a control.

23. The method of paragraphs 21 or 22, wherein binding is detected by an immunoassay, immunohistochemistry, Western blotting, surface plasmon resonance, flow cytometry (FACS) analysis, or a biochip.

24. The method of any one of paragraphs 21-23, wherein the immunoassay is selected from an enzyme immunoassay (EIA), radioimmunoassay (RIA), fluoroimmunoassay (FIA), chemiluminescent immunoassay (CLIA) and counting immunoassay (CIA), homogeneous enzyme-multiplied immunoassays (“EMIT”), apoenzyme reactivation immunoassay (“ARIS”), dipstick immunoassays, or immuno-chromatography assays.

25. The method of any one of paragraphs 21-24, wherein the biological sample is cells or a cell lysate or a fraction thereof.

26. The method of paragraph 25, wherein the cells or cell lysate or fraction thereof are derived from a biopsy from a subject.

27. The method of paragraph 26, wherein the biopsy contains or is suspected of containing tumor cells.

28. The method of paragraphs 27, wherein the biopsy is a tumor biopsy.

29. A method of diagnosing a subject with a Cdk5-related disease or disorder including detecting aberrant Cdk5 according to the method of any one of paragraphs 22-28.

30. The method of paragraph 29, wherein the Cdk5-related disease or disorder is cancer, diabetes, obesity, an immune system disorder, an inflammatory disease, an infectious disease, or a neurological disease optionally selected from Alzheimer's disease, Parkinson's disease, Huntington's disease, Cerebral ischemia, Traumatic brain injury, and addiction.

31. The method of paragraph 30, wherein the Cdk5-related disease or disorder is a cancer selected from neuroendocrine cancers, colorectal cancer, melanomas, lung cancer, head and neck squamous cell carcinoma, hepatocellular carcinoma (HCC), pancreatic cancer, breast cancer.

32. The method of paragraph 21, wherein the neuroendocrine cancer is Medullary Thyroid Carcinoma (MTC), pancreatic, adrenal, thyroid, and gastrointestinal cancer.

33. The method of any one of paragraphs 29-32 further including treating the subject.

34. The method of paragraph 33, wherein the treatment includes a therapy effective for treating diseases and disorders characterized by aberrant Cdk5 activity.

35. The method of paragraph 34, wherein the treatment includes administering the subject an effective amount of Dinaciclib (SCH727965), a proteasome inhibitor optionally Bortezomib (Velcade), Indolinone A (IndoA), MRT3-007, TFP5/TP5, or a functional nucleic acid that reduces expression of the Cdk5 gene, mRNA, or protein, optionally wherein the functional nucleic acid is siRNA, miRNA, RNAi, or shRNA.

The present invention will be further understood by reference to the following non-limiting examples.

### EXAMPLES

Cyclin-dependent kinase 5 (Cdk5), and its co-activators p35/25 have emerged as important molecular players in the tumorigenesis [9]. Aberrant activation of Cdk5 under the control of NE cell-specific promoter develops clinically accurate neuroendocrine tumors (NETs) [10-12]. In this study, a new Cdk5-driven model is established that exhibits early tumor onset and increased tumor volume doubling time compared to the previously established model. The model replicates the aggressive phenotype observed in humans.

### Material and Methods

### Generation of Transgenic MTC Models

The NSE-p25 bitransgenic mice were generated as described previously [13]. PiggyBac technology (Cyagen Biosciences) was used to generate single-copy CGRP transgenic mice in C57BL/6 background. Bitransgenic mice were then generated by crossing the TetOp-p25GFP mouse with that of CGRP-tTA or NSE-tTA. p25OE was controlled by doxycycline administration (Dox, 0.1 g/L) dissolved in drinking water. Doxycycline was removed at three weeks of age inducing tumors to grow for ~10 weeks. Tumors were arrested by re-administration of doxycycline. At the end of the experiments, bilateral tumors were harvested and snap-frozen for sequencing experiments, immunoblotting, and fixed for immunohistological staining. All mice were group-housed on a 12 h light/dark cycle with access to food and water ad libitum. All animal procedures were performed under protocols approved by the UAB Institutional Animal Care and Use Committee.

### Polymerase Chain Reaction

Positive pups carrying CGRP-tTA transgene confirmed by genotyping using primers-forward (F): ATCAAGAGTCACCGCCTCGC; Reverse (R): TTTGAGCGAGTTTCCTTGTCGTC. Transgene product size 215 bp. All positive pups were confirmed by PCR to not contain any integration of the helper plasmid. The pups carrying the NSE-tTA transgene were confirmed using the following primers—tTA 1080R: TTT CTG TAG GCC GTG TAC CTA; tTA 906F: GAT GTT AGA TAG GCG CCC TAC TCA C; Gdf5-D1: GGA GCA CTT CCA CTA TGG GAC & Gdf5-D2: AAA GAG TGA GGA GTT TGG GGA G. Transgene product size 243 bp. The Tet-op p25 gene was evaluated by the following primers—HS 18: CCA TCG ATC TAG TAC AGC TCG TCC ATG C; HS 28: AAG GAC GAC GGC AAC TAC; Gdf5-D1: GGA GCA CTT CCA CTA TGG GAC & Gdf5-D2: AAA GAG TGA GGA GTT TGG GGA G. Transgene product size 400 bp. Bitransgenic mice were positive for both the NSE or CGRP and p25-GFP alleles while control littermates were positive only for one of the two alleles. All reactions were carried out using the 2× master mix from Promega.

### Magnetic Resonance Imaging

MRI was performed with a Bruker Biospec 9.4 Tesla instrument using Paravision 5.1 software (Bruker Biospin, Billerica, MA). A Bruker 72 mm ID volume coil was used for excitation and a custom 24 mm surface coil for signal reception (Doty Scientific Inc., Columbia, SC). Mice were anesthetized with isoflurane gas and respiration observed with a MRI-compatible physiological monitoring system (SA Instruments Inc., Stony Brook, NY). Animals were imaged in supine position on a Bruker animal bed system with circulating heated water to maintain body temperature. A 2D T2-weighted RARE sequence was used for imaging of the abdomen. The following imaging parameters were used: TR/TE=2000/25 ms, echo spacing=12.5 ms, ETL=4, 2 averages, 29 contiguous axial slices with 1 mm thickness, FOV=30×30 mm and matrix=300×300 for an in-plane resolution of 100 μm. Prospective respiratory gating was used to minimize motion artifacts. Tumor volumes were quantitated using ImageJ software.

### Immunoblotting and Immunohistological Staining

Cells and tumor tissues were lysed in 1% SDS plus 50 mM NaF. Samples were sonicated briefly, spun at 20,000 g for 5 min, and supernatant combined with Laemmli buffer for analysis by SDS-PAGE followed by transfer to nitrocellulose membrane and subsequent detection of target proteins using a Li-Cor Odyssey imaging system. Immunoblotting was performed using antibodies for Cdk5 (Rockland 200-301-163; 1:1000), GFP (Cell Signaling Technology 2956; 1:2000), P-T202 LARP6 (disclosed herein; 1:1000), LARP6 (Invitrogen PA5-41881; 1:1000), P-S17 H1.5 (disclosed herein; 1:1000), H1.5 (Santa Cruz sc-247158; 1:1000), P-S988 RBL1 (disclosed herein; 1:1000), RBL1 (Santa Cruz sc-318; 1:500), P-S391 SUV39H1 (disclosed herein; 1:1000), SUV39H1 (sc-377112; 1:1000), P-T143 FAM53C (disclosed herein; 1:1000), FAM53C (Invitrogen PA5-114093; 1:1000) and β-actin (Invitrogen AM4302; 1:5000).

For immunostaining, tissues were fixed in formalin, embedded in paraffin, and sliced into 5 μm sections for placement on glass slides. Samples were deparaffinized and subjected to high temperature antigen retrieval in citrate buffer (pH 6.0). For IHC, samples were permeabilized in 0.3% Triton X-100, blocked with 5% normal goat serum, and then incubated overnight at 4° C. in primary antibodies to GFP (Cell Signaling 2956; 1:200), ChrA (Abcam ab15160; 1:1000), P-T202 LARP6 (disclosed herein; 1:200), P-S17 H1.5 (disclosed herein; 1:50), and P-S988 RBL1 (disclosed herein; 1:100) diluted in 5% normal goat serum and 0.3% Tween 20. Sections were then incubated in 0.3% hydrogen peroxide and biotinylated secondary antibodies (Pierce 31820 or 31800; 1:500) applied to slides for 1 h at room temperature followed by 30 min of streptavidin-HRP. Slides were then incubated with DAB Chromogen (Dako Liquid DAB+ substrate K3468) and counter-stained with hematoxylin. Standard procedures were used for H&E staining (Feldman and Wolfe, Methods Mol Biol. 2014; 1180:31-43. doi: 10.1007/978-1-4939-1050-2_3. PMID: 25015141.). Archival human tissues and tissue microarrays were obtained in accordance with UAB IRB protocol IRB-300002147. Stains of human tissues were reviewed for quality by a board-certified pathologist with expertise in thyroid pathology.

### Whole Exome Sequencing (WES) and RNA Sequencing (RNA-Seq)

Total DNA was extracted from the frozen tumors using DNeasy Blood and tissue kit (Qiagen) according to the manufacturer's instructions, and RNA was purified using RNEasy Plus Mini Kit (Qiagen). For WES, exome capture was performed using the Agilent SureSelect Mouse All Exome QXT capture kit (Agilent). Briefly, the genomic DNA was subjected to tagmentation reactions inserting adaptor sequences randomly throughout the genome. The DNA was PCR amplified and then incubated with biotin labeled RNA capture probes complementary to every exon. Following purification of the exome sequences through streptavidin-magnetic bead separation, the DNA was amplified with primers that introduced 8-nucleotide index so that separate samples can run in the same lane for sequence analysis. The exomic libraries were run on the NextSeq500 next generation sequencer from Illumina (Illumina, San Diego, CA) with paired end 75 bp reads using standard techniques. RNA-seq was performed on the same instrument. Briefly, RNA quality was assessed using the Agilent 2100 Bioanalyzer. RNA Integrity Number (RIN) of ≥7.0 was used for sequencing library preparation. Quality controlled RNA was converted to a sequencing ready library using the NEBNext Ultra II Directional RNA library kit with polyA selection as per the manufacturer's instructions (New England Biolabs). The cDNA libraries were quantitated using qPCR in a Roche LightCycler 480 with the Kapa Biosystems kit for Illumina library quantitation (Kapa Biosystems, Woburn, MA) before cluster generation.

### Bioinformatics Analysis

Exome Seq—MoCaSeq pipeline was used to analyze raw WES data (source code: github.com/roland-rad-lab/MoCaSeq)[14]. Using Docker and Ubuntu Linux, the pipeline was set up. With Trimmomatic (v0.38) [15] and BWA-MEM (v0.7.17)[16], the raw reads were aligned to the mouse reference genome GRCm38.p6. For further post-processing, Picard 2.20.0 and GATK (v4.1.0.0) were used [17]. The cutoff of the variant allele frequency was set at ≥10% as recommended [14]. For the loss of heterozygosity (LOH) analyses from WES data, somatic SNP calling was performed using Mutect2[18]. LOH analyses were limited to reads with a mapping quality of 60 to avoid ambiguous SNP positions caused by mis-mapping. CopywriteR (v2.6.1.216) [19], which extracts DNA copy number information from off-target reads, was used to call CNVs. Custom Python (v.3.10) and Shell scripts were used for downstream analysis and visualization. RNA Seq—To remove low-quality reads from raw sequences, fastp (v0.21.0) was used [20]. Sequence alignment was performed using STAR v2.7.3a-GCC-6.4.0-2.28 aligner[21] and GRCm39 assembly. Using the accepted alignment hits, gene counts were obtained using HTSeq (HTSeq v0.12.3-foss-2018b-Python-3.6.6)[22]. The differentially expressed genes (1.5<=Fold change and 0.05=>FDR) were identified using DESeq2 (DESeq2_1.36.0) [23] and R (v4.2.0). The enriched gene sets and pathways were analyzed using GSEApy (0.13.0), Enrichr, Shiny GO, and Metascape.

### Cell Proliferation

Cell proliferation assay was performed on mouse MTC cells[10] in the presence or absence of doxycycline using CyQUANT™ Direct Cell Proliferation Assay following the manufacturer's protocol (Thermo Fisher Scientific)[24].

### Generation and Purification of Monoclonal Antibodies

The rabbit monoclonal antibodies were developed directly from isolated B cells of immunized animals without the use of hybridomas. Briefly, an antigen peptide containing the phosphorylated site of interest is synthesized with an N-terminal cysteine and conjugated via the thiol-group to carrier proteins. At least two rabbits are immunized with the peptide. After immunization and subsequent boosts, peripheral blood is drawn and the titer of the antiserum against the antigen is determined via indirect ELISA assays against the phosphorylated peptide. The rabbit with the highest titer and desired activities was used for the isolation of peripheral blood mononuclear cells (PBMCs). Antigen-specific B cells are cultured in vitro in multi-well plates and supernatant samples are screened to identify desired antibodies. The cDNAs encoding the heavy (H) and light (L) chains of the antibodies were obtained by reverse transcription-polymerase chain reaction (RT-PCR) of RNA samples isolated from positive B cell clones. H and L cDNAs cloned into mammalian expression vectors and were transiently transfected into Chinese hamster ovary (CHO) cells. Recombinant monoclonal antibodies were generated from the expressed heavy and light chains. Before purification, recombinant antibodies were screened by ELISA and additional application assays. For antibodies that are specific for phosphorylated sites on proteins, the non-phosphorylated antigens were used for counter-screening assays (monoclonal rabbit antibodies by Excel BioPharm LLC).

### DNA and Protein Sequences of Final Selected Clones

cDNAs encoding H- and K-chain of selected IgGs were cloned into the XbaI and EcoRV sites of plasmid vector pcDNA3.4. The XbaI site was destroyed.

Detailed information on pcDNA3.4 vector is available on Thermo website: tools.thermofisher.com/content/sfs/manuals/pcdna3_4_topo_ta_cloning_kit_man.pdf

In the sequences below, the variable regions are denoted with italics (also separately provided above), the CDRs are bolded (also separately provided above), and the cleavable signal sequence is underlined.

### RB1-1

```
>Clone pEB1930P-1C4-H1 in pcDNA3.4
. . . (pcDNA3.4) TCCGGACTCTAGCACC (SEQ ID NO: 68)
                                BspEI XbaI (partial)
ATGGAGACTGGGCTGCGCTGGCTTCTCCTGGTCGCTGTGCTCAAAGGTGTCCAGTGTCAGTCGCT
GGAGGAGTCCGGGGGTCGCCTGGTCACGCCTGGGACACCCCTGAAACTCACCTGCACAGTCTCTG
GATTCTCCCTCAGTGACTACAACGTGGGCTGGGTCCGCCAGGCTCCAGGGAAGGGGCTGGAATGG
ATCGGAATCATGAATATTGGIATTAGCACATGGTACGCGAGCTGGGCAAAAGGCCGATTCACCAT
CTCCAGAACCTCGACCACGGTGGATCTGAAAATGACCAGTCTGACAACCGAGGACACGGCCACCT
ATTTCTGTGCCAGAGGGTTTAGTCGTAATAGTTATGATATATGGGGCCCAGGCACCCTGGTCACC
GTCTCCTCAGGGCAACCTAAGGCTCCATCAGTCTTCCCACTGGCCCCCTGCTGCGGGGACACACC
CAGCTCCACGGTGACCCTGGGCTGCCTGGTCAAAGGCTACCTCCCGGAGCCAGTGACCGTGACCT
GGAACTCGGGCACCCTCACCAATGGGGTACGCACCTTCCCGTCCGTCCGGCAGTCCTCAGGCCTC
TACTCGCTGAGCAGCGTGGTGAGCGTGACCTCAAGCAGCCAGCCCGTCACCTGCAACGTGGCCCA
CCCAGCCACCAACACCAAAGTGGACAAGACCGTTGCGCCCTCGACATGCAGCAAGCCCATGTGCC
CACCCCCTGAACTCCTGGGGGGACCGTCTGTCTTCATCTTCCCCCCAAAACCCAAGGACACCCTC
ATGATCTCACGCACCCCCGAGGTCACATGCGTGGTGGTGGACGTGAGCCAGGATGACCCCGAGGT
GCAGTTCACATGGTACATAAACAACGAGCAGGTGCGCACCGCCCGGCCGCCGCTACGGGAGCAGC
AGTTCAACAGCACGATCCGCGTGGTCAGCACCCTCCCCATCGCGCACCAGGACTGGCTGAGGGGC
AAGGAGTTCAAGTGCAAAGTCCACAACAAGGCACTCCCGGCCCCCATCGAGAAAACCATCTCCAA
AGCCAGAGGGCAGCCCCTGGAGCCGAAGGTCTACACCATGGGCCCTCCCCGGGAGGAGCTGAGCA
GCAGGTCGGTCAGCCTGACCTGCATGATCAACGGCTTCTACCCTTCCGACATCTCGGTGGAGTGG
GAGAAGAACGGGAAGGCAGAGGACAACTACAAGACCACGCCGGCCGTGCTGGACAGCGACGGCTC
CTACTTCCTCTACAGCAAGCTCTCAGTGCCCACGAGTGAGTGGCAGCGGGGCGACGTCTTCACCT
GCTCCGTGATGCACGAGGCCTTGCACAACCACTACACGCAGAAGTCCATCTCCCGCTCTCCGGG
TAAATGA
(SEQ ID NO: 12)
GCGCTGTGCCGGCGATATC (SEQ ID NO: 69) (pcDNA3.4) . . .
                          EcoRV
Translated H-Chain with CDRs Marked
```

| | |
|---|---|
| METGLRWLLLVAVLKGVQCQSLEESGGRLVTPGTPLKLTCTVSGFSLSDY | 50 |
| NVGWVRQAPGKGLEWIGIMNIGISTWYASWAKGRFTISRTSTTVDLKMTS | 100 |
| LTTEDTATYFCARGFSRNSYDIWGPGTLVTVSSGQPKAPSVFPLAPCCGD | 150 |
| TPSSTVTLGCLVKGYLPEPVTVTWNSGTLINGVRTFPSVRQSSGLYSLSS | 200 |
| VVSVTSSSQPVTCNVAHPATNTKVDKTVAPSTCSKPMCPPPELLGGPSVF | 250 |
| IFPPKPKDTLMISRTPEVTCVVVDVSQDDPEVQFTWYINNEQVRTARPPL | 300 |
| REQQFNSTIRVVSTLPIAHQDWLRGKEFKCKVHNKALPAPIEKTISKARG | 350 |
| QPLEPKVYTMGPPREELSSRSVSLTCMINGFYPSDISVEWEKNGKAEDNY | 400 |
| KTTPAVLDSDGSYFLYSKLSVPTSEWQRGDVFTCSVMHEALHNHYTQKSI | 450 |
| SRSPGK* (SEQ ID NO: 10) | |

```
>Clone pEB1930P-1C4-K1 in pcDNA3.4
. . . (pcDNA3.4) TCCGGACTCTAGCACC (SEQ ID NO: 68)
                            BspEI XbaI (partial)
ATGGACACGAGGGCCCCCACTCAGCTGCTGGGGCTCCTGCTGCTCTGGCTCCCAGGTGCCACATT
TGCCGCCGTGCTGACCCAGACTCCATCTCCCGTGTCTGCAGCTGTGGGAGGCACAGTCACCATCA
AGTGCCAGTCCAGTCAGAGTGTTGTTAAGAACAACTACTTATCCTGGTATCAGCAGAAACCAGGG
CAACCTCCCAAACTCCTGATCTACGAAACATCCAAACTGGCATCTGGGGTCCCATCGCGGTTCAA
AGGCAGTGGATCTGGGACACAGTTCACTCTCACCATCAGCGACGTGCAGTGTGACGATGCTGCCA
CTTACTACTGTGCAGGCGGTTATAGTAGTATTAGTGATACTACTTTCGGCGGAGGGACCGAGGTG
GTGGTCAAAGGTGATCCAGTTGCACCTACTGTCCTCATCTTCCCACCAGCTGCTGATCAGGTGGC
AACTGGAACAGTCACCATCGTGTGTGTGGCGAATAAATACTTTCCCGATGTCACCGTCACCTGGG
AGGTGGATGGCACCACCCAAACAACTGGCATCGAGAACAGTAAAACACCGCAGAATTCTGCAGAT
TGTACCTACAACCTCAGCAGCACTCTGACACTGACCAGCACACAGTACAACAGCCACAAAGAGTA
CACCTGCAAGGTGACCCAGGGCACGACCTCAGTCGTCCAGAGCTTCAATAGGGGTGACTGT TAG
(SEQ ID NO: 13)
AGCGATATC (pcDNA3.4) . . .
      EcoRV
Translated K-Chain with CDRs Marked
```

| | |
|---|---|
| MDTRAPTQLLGLLLLWLPGATFAAVLTQTPSPVSAAVGGTVTIKCQSSQS | 50 |
| VVKNNYLSWYQQKPGQPPKLLIYETSKLASGVPSRFKGSGSGTQFTLTIS | 100 |
| DVQCDDAATYYCAGGYSSISDTTFGGGTEVVVKGDPVAPTVLIFPPAADQ | 150 |
| VATGTVTIVCVANKYFPDVTVTWEVDGTTQTTGIENSKTPQNSADCTYNL | 200 |
| SSTLTLTSTQYNSHKEYTCKVTQGTTSVVQSFNRGDC* | |

```
(SEQ ID NO: 11)
***
H1-5-1
>Clone pEB1931P-1B6-H2 in pcDNA3.4
. . . (pcDNA3.4) TCCGGACTCTAGCACC (SEQ ID NO: 68)
                            BspEI XbaI (partial)
ATGGAGACTGGGCTGCGCTGGCTTCTCCTGGTCGCTGTGCTCAAAGGTGTCCAGTGTCAGTCGGT
GGAGGAGTCCGGGGGTCGCCTGGTCACGCCTGGGACACCCCTGACACTCACCTGCACAGTCTCTG
GAATCGACCTCAGTAGCAATGTAATGATGTGGGTCCGCCAGGCTCCAGGGAAGGGGCTGGAATAC
ATCGGAATCATTACTAATAGTGGTATTAGATACTACGCGAGCTGGGCGAAAGGCCGATTCACCAT
CTCCAAAACCTCGACCACAGTGGATCTGAAAATCACCAGTCCGACAACCGAGGACACGGCCACCT
ATTTCTGTGCCAGAGGGGCTCCTAATACTGGTAACATCTGGGGCCCAGGCACCCTGGTCACCGTC
TCCTTAGGGCAACCTAAGGCTCCATCAGTCTTCCCACTGGCCCCCTGCTGCGGGGACACACCCAG
CTCCACGGTGACCCTGGGCTGCCTGGTCAAAGGCTACCTCCCGGAGCCAGTGACCGTGACCTGGA
ACTCGGGCACCCTCACCAATGGGGTACGCACCTTCCCGTCCGTCCGGCAGTCCTCAGGCCTCTAC
TCGCTGAGCAGCGTGGTGAGCGTGACCTCAAGCAGCCAGCCCGTCACCTGCAACGTGGCCCACCC
AGCCACCAACACCAAAGTGGACAAGACCGTTGCGCCCTCGACATGCAGCAAGCCCATGTGCCCAC
CCCCTGAACTCCTGGGGGGACCGTCTGTCTTCATCTTCCCCCCAAAACCCAAGGACACCCTCATG
ATCTCACGCACCCCCGAGGTCACATGCGTGGTGGTGGACGTGAGCCAGGATGACCCCGAGGTGCA
GTTCACATGGTACATAAACAACGAGCAGGTGCGCACCGCCCGGCCGCCGCTACGGGAGCAGCAGT
TCAACAGCACGATCCGCGTGGTCAGCACCCTCCCCATCGCGCACCAGGACTGGCTGAGGGGCAAG
GAGTTCAAGTGCAAAGTCCACAACAAGGCACTCCCGGCCCCCATCGAGAAAACCATCTCCAAAGC
CAGAGGGCAGCCCCTGGAGCCGAAGGTCTACACCATGGGCCCTCCCCGGGAGGAGCTGAGCAGCA
GGTCGGTCAGCCTGACCTGCATGATCAACGGCTTCTACCCTTCCGACATCTCGGTGGAGTGGGAG
AAGAACGGGAAGGCAGAGGACAACTACAAGACCACGCCGGCCGTGCTGGACAGCGACGGCTCCTA
CTTCCTCTACAGCAAGCTCTCAGTGCCCACGAGTGAGTGGCAGCGGGGCGACGTCTTCACCTGCT
CCGTGATGCACGAGGCCTTGCACAACCACTACACGCAGAAGTC
CATCTCCCGCTCTCCGGGTAAATGA
(SEQ ID NO: 25)
GCGCTGTGCCGGCGATATC (SEQ ID NO: 69) (pcDNA3. 4) . . .
                                    EcoRV
Translated H-Chain with CDRs Marked
```

| | |
|---|---|
| METGLRWLLLVAVLKGVQCQSVEESGGRLVTPGTPLILTCTVSGIDLSSN | 50 |
| VMMWVRQAPGKGLEYIGIITNSGIRYYASWAKGRFTISKTSTTVDLKITS | 100 |
| PTTEDTATYFCARGAPNTGNIWGPGTLVTVSLGQPKAPSVFPLAPCCGDT | 150 |
| PSSTVTLGCLVKGYLPEPVTVTWNSGTLINGVRTFPSVRQSSGLYSLSSV | 200 |
| VSVTSSSQPVTCNVAHPATNTKVDKTVAPSTCSKPMCPPPELLGGPSVFI | 250 |
| FPPKPKDTLMISRTPEVTCVVVDVSQDDPEVQFTWYINNEQVRTARPPLR | 300 |
| EQQFNSTIRVVSTLPIAHQDWLRGKEFKCKVHNKALPAPIEKTISKARGQ | 350 |
| PLEPKVYTMGPPREELSSRSVSLTCMINGFYPSDISVEWEKNGKAEDNYK | 400 |
| TTPAVLDSDGSYFLYSKLSVPTSEWQRGDVFTCSVMHEALHNHYTQKSIS | 450 |
| RSPGK* | |

```
(SEQ ID NO: 23)
>Clone pEB1931P-1B6-K2 in pcDNA3.4
. . . (pcDNA3.4) TCCGGACTCTAGCACC (SEQ ID NO: 68)
                            BspEI XbaI (partial)
ATGGACACGAGGGCCCCCACTCAGCTGCTGGGGCTCCTGCTGCTCTGGCTCCCAGGTGCCACATT
TGCGCAAGTGCTGACCCAGACTGCATCGTCCGTGTCTGCAGCTGTGGGAGGCACAGTCACCATCA
ATTGCCAGTCCAGTCAGAGTGTTTATGATAACAACTACTTATCCTGGTATCAACAGAAACCAGGG
CAGCCTCCCAAGCTCCTGATCTACCAGGCATCCAAACTGGCATCIGGGGTCCCATCGCGGITCAA
AGGCAGTGGATCTGGGACACAGTTCACTCTCACCATCAGCGACCTGGAGTGTGACGATGCTGTCA
CTTACTACTGTGCAGGCGCTTATTTTGGTAATATTTATACTTTCGGCGGAGGGACCGAGGTGGTG
GTCAAAGGTGATCCAGTTGCACCTACTGTCCTCATCTTCCCACCAGCTGCTGATCAGGTGGCAAC
TGGAACAGTCACCATCGTGTGTGTGGCGAATAAATACTTTCCCGATGTCACCGTCACCTGGGAGG
TGGATGGCACCACCCAAACAACTGGCATCGAGAACAGTAAAACACCGCAGAATTCTGCAGATTGT
ACCTACAACCTCAGCAGCACTCTGACACTGACCAGCACACAGTACAACAGCCACAAAGAGTACAC
CTGCAAGGTGACCCAGGGCACGACCTCAGTCGTCCAGAGCTTCAATAGGGGTGACTGTTAG
(SEQ ID NO: 26)
AGCGATATC (SEQ ID NO: 70) (pcDNA3. 4) . . .
        EcoRV
Translated K-Chain with CDRs Marked
```

| | |
|---|---|
| MDTRAPTQLLGLLLLWLPGATFAQVLTQTASSVSAAVGGTVTINCQSSQS | 50 |
| VYDNNYLSWYQQKPGQPPKLLIYQASKLASGVPSRFKGSGSGTQFTLTIS | 100 |
| DLECDDAVTYYCAGAYFGNIYTFGGGTEVVVKGDPVAPTVLIFPPAADQV | 150 |
| ATGTVTIVCVANKYFPDVTVTWEVDGTTQTTGIENSKTPQNSADCTYNLS | 200 |
| STLTLTSTQYNSHKEYTCKVTQGTTSVVQSFNRGDC* | |

(SEQ ID NO: 24)
***
LARP6-1
>Clone pEB1932P-1D5-H2 in pcDNA3.4
. . . (pcDNA3.4)TCCGGACTCTAGCACC (SEQ ID NO: 68)
BspEI XbaI (partial)
ATGGAGACTGGGCTGCGCTGGCTTCTCCTGGTCGCTGTGCTCAAAGGTGTCCAGTGTCAGTCGTT
GGAGGAGTCCGGGGGAGGCCTGGTCCAGCCTGAGGGATCCCTGACACTCACCTGCAAAGCCTCTG
GATTCTCCTTCAGTAGCGGCTACTACATGTGCTGGGTCCGCCAGGCTCCAGGGAAGGGGCTGGAG
TGGAGCGGTTGCATTAGTGCTCGTAGTGGTAGAACTTACTACGCGACCTGGGCGAAAGGCCGATT
CACCATCTCCAAAACCTCGTCGACCACGGTGACTCTGCAAGTGACCAGTCTGACAGCCGCGGACA
CGGCCACCTATTTCTGTGCGAGAGGGAATAGATTTGTTAGTAGTAGTGGTGATTCCATGTGGGGC
CCAGGCACCCTGGTCACCGTCTCCTCAGGGCAACCTAAGGCTCCATCAGTCTTCCCACTGGCCCC
CTGCTGCGGGGACACACCCAGCTCCACGGTGACCCTGGGCTGCCTGGTCAAAGGCTACCTCCCGG
AGCCAGTGACCGTGACCTGGAACTCGGGCACCCTCACCAATGGGGTACGCACCTTCCCGTCCGTC
CGGCAGTCCTCAGGCCTCTACTCGCTGAGCAGCGTGGTGAGCGTGACCTCAAGCAGCCAGCCCGT
CACCTGCAACGTGGCCCACCCAGCCACCAACACCAAAGTGGACAAGACCGTTGCGCCCTCGACAT
GCAGCAAGCCCATGTGCCCACCCCCTGAACTCCTGGGGGGACCGTCTGTCTTCATCTTCCCCCCA
AAACCCAAGGACACCCTCATGATCTCACGCACCCCCGAGGTCACATGCGTGGTGGTGGACGTGAG
CCAGGATGACCCCGAGGTGCAGTTCACATGGTACATAAACAACGAGCAGGTGCGCACCGCCCGGC
CGCCGCTACGGGAGCAGCAGTTCAACAGCACGATCCGCGTGGTCAGCACCCTCCCCATCGCGCAC
CAGGACTGGCTGAGGGGCAAGGAGTTCAAGTGCAAAGTCCACAACAAGGCACTCCCGGCCCCCAT
CGAGAAAACCATCTCCAAAGCCAGAGGGCAGCCCCTGGAGCCGAAGGTCTACACCATGGGCCCTC
CCCGGGAGGAGCTGAGCAGCAGGTCGGTCAGCCTGACCTGCATGATCAACGGCTTCTACCCTTCC
GACATCTCGGTGGAGTGGGAGAAGAACGGGAAGGCAGAGGACAACTACAAGACCACGCCGGCCGT
GCTGGACAGCGACGGCTCCTACTTCCTCTACAGCAAGCTCTCAGTGCCCACGAGTGAGTGGCAGC
GGGGCGACGTCTTCACCTGCTCCGTGATGCACGAGGCCTTGCACAACCACTACACGCAGAAGTC
CATCTCCCGCTCTCCGGGTAAATGA
(SEQ ID NO: 38)
GCGCTGTGCCGGCGATATC (SEQ ID NO: 69) (pcDNA3. 4) . . .
EcoRV
Translated H-Chain with CDRs Marked

| | |
|---|---|
| METGLRWLLLVAVLKGVQCQSLEESGGGLVQPEGSLTLTCKASGFSFSSG | 50 |
| YYMCWVRQAPGKGLEWSGCISARSGRTYYATWAKGRFTISKTSSTTVTLQ | 100 |
| VTSLTAADTATYFCARGNRFVSSSGDSMWGPGTLVTVSSGQPKAPSVFPL | 150 |
| APCCGDTPSSTVTLGCLVKGYLPEPVTVTWNSGTLINGVRTFPSVRQSSG | 200 |
| LYSLSSVVSVTSSSQPVTCNVAHPATNTKVDKTVAPSTCSKPMCPPPELL | 250 |
| GGPSVFIFPPKPKDTLMISRTPEVTCVVVDVSQDDPEVQFTWYINNEQVR | 300 |
| TARPPLREQQFNSTIRVVSTLPIAHQDWLRGKEFKCKVHNKALPAPIEKT | 350 |
| ISKARGQPLEPKVYTMGPPREELSSRSVSLTCMINGFYPSDISVEWEKNG | 400 |
| KAEDNYKTTPAVLDSDGSYFLYSKLSVPTSEWQRGDVFTCSVMHEALHNH | 450 |
| YTQKSISRSPGK* | |

```
(SEQ ID NO: 36)
>Clone pEB1932P-1D5-K1 in pcDNA3.4
. . . (pcDNA3.4) TCCGGACTCTAGCACC (SEQ ID NO: 68)
                            BspEI XbaI (partial)
ATGGACACGAGGGCCCCCACTCAGCTGCTGGGGCTCCTGCTGCTCTGGCTCCCAGGTGCCATATG
TGACCCTGTGCTGACCCAGACTCCATCCCCTGTGTCTGCAGCTGTGGGAGGCACAGTCACCATCA
ATTGCCAGGCCAGTCAGAGTGTTTTTAGTAACAACCAACTAGCCTGGTTTCAGCAGAAACCAGGG
CAGCCTCCCAAGCAACTGATCTATGGIGCATCCACTCIGGCATCTGGGGTCTCATCGCGGTTCAA
AGGCAGTGGATATGGGACACGGTTCACTCTCACCATCAGCGACGTGCAGTGTGACGATACTGCCA
CTTACTACTGTCTAGGCGAATTTACTTGTAGTAGTGTTGATTGTAATGCTTTTGGCGGAGGGACC
GAGGTGGTCGTCGAGGGTGATCCAGTTGCACCTACTGTCCTCATCTTCCCACCATCTGCTGATCT
TGTGGCAACTGGAACAGTCACCATCGTGTGTGTGGCGAATAAATACTTTCCCGATGTCACCGTCA
CCTGGGAGGTGGATGGCACCACCCAAACAACTGGCATCGAGAACAGTAAAACACCGCAGAATTCT
GCAGATTGTACCTACAACCTCAGCAGCACTCTGACACTGACCAGCACACAGTACAACAGCCACAA
AGAGTACACCTGCAAGGTGACCCAGGGCACGACCTCAGTCGTCCAGAGCTTCAATAGGGGTGACT
GTTAG (SEQ ID NO: 39)
AGCGATATC (SEQ ID NO: 70) (pcDNA3.4) . . .
      EcoRV
Translated K-Chain with CDRs Marked
```

| | |
|---|---|
| MDTRAPTQLLGLLLLWLPGAICDPVLTQTPSPVSAAVGGTVTINCQASQS | 50 |
| VFSNNQLAWFQQKPGQPPKQLIYGASTLASGVSSRFKGSGYGTRFTLTIS | 100 |
| DVQCDDTATYYCLGEFTCSSVDCNAFGGGTEVVVEGDPVAPTVLIFPPSA | 150 |
| DLVATGTVTIVCVANKYFPDVTVTWEVDGTTQTTGIENSKTPQNSADCTY | 200 |
| NLSSTLTLTSTQYNSHKEYTCKVTQGTTSVVQSFNRGDC* | |

```
(SEQ ID NO: 37)
***
SUV39H1-1
>Clone pEB1933P-2C8-H2 in pcDNA3.4
....
....
. . . (pcDNA3.4) TCCGGACTCTAGCACC (SEQ ID NO: 68)
                            BspEI XbaI (partial)
ATGGAGACTGGGCTGCGCTGGCTTCTCCTGGTCGCTGIGCICAAAGGTGTCCAGTGTCAGTCGGT
GGAGGAGTCCGGGGGTCGCCTGGTCACGCCTGGGACACCCCTGACACTCACCTGCACAGTCTCTG
GATTCTCCCTAAGTACTTACCACATGTGCTGGGTCCGCCAGGCTCCAGGGAAGGGGCTGGAATAC
ATCGGAATGATTAATAGGCGTGCTATTACATCCTACGCGAGCTGGGCGAAAGGCCGATTCACCAT
CTCCAAAACCTCGACCACGGTGGATCTGAAAATCACCAGTCCGACAACCGAGGACACGGCCACCT
ATTTCTGTGCCAGATATAGTAGTGGTAATGATTTTGATGCGGACATCIGGGGCCCAGGCACCCTG
GTCACCGTCTCCTTAGGGCAACCTAAGGCTCCATCAGTCTTCCCACTGGCCCCCTGCTGCGGGGA
CACACCCAGCTCCACGGTGACCCTGGGCTGCCTGGTCAAAGGCTACCTCCCGGAGCCAGTGACCG
TGACCTGGAACTCGGGCACCCTCACCAATGGGGTACGCACCTTCCCGTCCGTCCGGCAGTCCTCA
GGCCTCTACTCGCTGAGCAGCGTGGTGAGCGTGACCTCAAGCAGCCAGCCCGTCACCTGCAACGT
GGCCCACCCAGCCACCAACACCAAAGTGGACAAGACCGTTGCGCCCTCGACATGCAGCAAGCCCA
TGTGCCCACCCCCTGAACTCCTGGGGGGACCGTCTGTCTTCATCTTCCCCCCAAAACCCAAGGAC
ACCCTCATGATCTCACGCACCCCCGAGGTCACATGCGTGGTGGTGGACGTGAGCCAGGATGACCC
CGAGGTGCAGTTCACATGGTACATAAACAACGAGCAGGTGCGCACCGCCCGGCCGCCGCTACGGG
AGCAGCAGTTCAACAGCACGATCCGCGTGGTCAGCACCCTCCCCATCGCGCACCAGGACTGGCTG
AGGGGCAAGGAGTTCAAGTGCAAAGTCCACAACAAGGCACTCCCGGCCCCCATCGAGAAAACCAT
CTCCAAAGCCAGAGGGCAGCCCCTGGAGCCGAAGGTCTACACCATGGGCCCTCCCCGGGAGGAGC
TGAGCAGCAGGTCGGTCAGCCTGACCTGCATGATCAACGGCTTCTACCCTTCCGACATCTCGGTG
GAGTGGGAGAAGAACGGGAAGGCAGAGGACAACTACAAGACCACGCCGGCCGTGCTGGACAGCGA
CGGCTCCTACTTCCTCTACAGCAAGCTCTCAGTGCCCACGAGTGAGTGGCAGCGGGGCGACGTCT
TCACCTGCTCCGTGATGCACGAGGCCTTGCACAACCACTACACGCAGAAGTCCATCTCCCGCTC
TCCGGGTAAATGA
(SEQ ID NO: 51)
GCGCTGTGCCGGCGATATC (SEQ ID NO: 69) (pcDNA3. 4) . . .
                                EcoRV
Translated H-Chain with CDRs Marked
```

| | |
|---|---|
| METGLRWLLLVAVLKGVQCQSVEESGGRLVTPGTPLTLTCTVSGFSLSTY | 50 |
| HMCWVRQAPGKGLEYIGMINRRAITSYASWAKGRFTISKTSTTVDLKITS | 100 |
| PTTEDTATYFCARYSSGNDFDADIWGPGTLVTVSLGQPKAPSVFPLAPCC | 150 |
| GDTPSSTVTLGCLVKGYLPEPVTVTWNSGTLINGVRTFPSVRQSSGLYSL | 200 |
| SSVVSVTSSSQPVTCNVAHPATNTKVDKTVAPSTCSKPMCPPPELLGGPS | 250 |
| VFIFPPKPKDTLMISRTPEVTCVVVDVSQDDPEVQFTWYINNEQVRTARP | 300 |
| PLREQQFNSTIRVVSTLPIAHQDWLRGKEFKCKVHNKALPAPIEKTISKA | 350 |
| RGQPLEPKVYTMGPPREELSSRSVSLTCMINGFYPSDISVEWEKNGKAED | 400 |
| NYKTTPAVLDSDGSYFLYSKLSVPTSEWQRGDVFTCSVMHEALHNHYTQK | 450 |
| SISRSPGK* | |

```
(SEQ ID NO: 49)
>Clone pEB1933P-2C8-K2 in pcDNA3.4
. . . (pcDNA3.4) TCCGGACTCTAGCACC (SEQ ID NO: 68)
                            BspEI XbaI (partial)
ATGGACACGAGGGCCCCCACTCAGCTGCIGGGGCTCCTGCTGCTCTGGCTCCCAGGTGCCAGATG
TGCCGCCGTGCTGACCCAGACTCCATCTCCCGTGTCTGCAGCTGTGGGAGGCACAGTCACCATCA
GTTGCCAGTCCAGTAAGAGTGTTTATGATAGAAACCTCTTATCCTGGTTTCAGCAGAAACCAGGG
CAGCCTCCCAAGCTCCTGATCTACAAGGCTTCCACTCTGGCATCTGGGGTCCCATCGCGGTTCAA
AGGCAGTGGATCTGGGACACAGTTCACTCTCACCATCAGCGACGTGCAGTGTGACGATGCTGCCA
CTTACTACTGTGCAGGCGGTTATAGTGGTACTAGTGATGCTTATCCTTTCGGCGGAGGGACCGAG
GTGGTGGTCAAAGGTGATCCAGTTGCACCTACTGTCCTCATCTTCCCACCAGCTGCTGATCAGGT
GGCAACTGGAACAGTCACCATCGTGTGTGTGGCGAATAAATACTTTCCCGATGTCACCGTCACCI
GGGAGGTGGATGGCACCACCCAAACAACTGGCATCGAGAACAGTAAAACACCGCAGAATTCTGCA
GATTGTACCTACAACCTCAGCAGCACTCTGACACTGACCAGCACACAGTACAACAGCCACAAAGA
GTACACCTGCAAGGTGACCCAGGGCACGACCTCAGTCGTCCAGAGCTTCAATAGGGGTGACTGTT
AG
(SEQ ID NO: 52)
AGCGATATC (SEQ ID NO: 70) (pcDNA3.4) . . .
        EcoRV
Translated K-Chain with CDRs Marked
```

| | |
|---|---|
| MDTRAPTQLLGLLLLWLPGARCAAVLTQTPSPVSAAVGGTVTISCQSSKS | 50 |
| VYDRNLLSWFQQKPGQPPKLLIYKASTLASGVPSRFKGSGSGTQFTLTIS | 100 |
| DVQCDDAATYYCAGGYSGTSDAYPFGGGTEVVVKGDPVAPTVLIFPPAAD | 150 |
| QVATGTVTIVCVANKYFPDVTVTWEVDGTTQTTGIENSKTPQNSADCTYN | 200 |
| LSSTLTLTSTQYNSHKEYTCKVTQGTTSVVQSFNRGDC* | |

```
(SEQ ID NO: 50)
***
FAM53C-1
>Clone pEB1934P-4A5-H2 in pcDNA3.4
. . . (pcDNA3.4) TCCGGACTCTAGCACC (SEQ ID NO: 68)
                          BspEI XbaI (partial)
ATGGAGACTGGGCTGCGCTGGCTTCTCCTGGTCGCTGTGCTCAAAGGTGTCCTGTGTCAGTCGTT
GGAGGAGTCCGGGGGAGACCTGGTCAAGCCTGGGGCATCCCTGACACTCACCTGCAAAGGCTCTG
GATTCTCCTTCACTATCAGATACAACATCTGCTGGGTCCGCCAGGCTCCAGGGAAGGGGCTGGAG
TGGATCGCATGTGTTAATAGTGCCTACGCGAGCTGGGCGAAAGGCCGATTCACCATCTCCAAAAC
CTCGTCGACCACGGTGACTCTGCAAATGACCAGTCTGACAGCCGCGGACACGGCCACCTATTTCT
GTGTGAGATATGTTGATAGTCGTTACTACGGCGTGGACCTCTGGGGCCCAGGGACCCTCGTCACC
GTCTCTTCAGGGCAACCTAAGGCTCCATCAGTCTTCCCACTGGCCCCCTGCTGCGGGGACACACC
CAGCTCCACGGTGACCCTGGGCTGCCTGGTCAAAGGCTACCTCCCGGAGCCAGTGACCGTGACCT
GGAACTCGGGCACCCTCACCAATGGGGTACGCACCTTCCCGTCCGTCCGGCAGTCCTCAGGCCTC
TACTCGCTGAGCAGCGTGGTGAGCGTGACCTCAAGCAGCCAGCCCGTCACCTGCAACGTGGCCCA
CCCAGCCACCAACACCAAAGTGGACAAGACCGTTGCGCCCTCGACATGCAGCAAGCCCATGTGCC
CACCCCCTGAACTCCTGGGGGGACCGTCTGTCTTCATCTTCCCCCCAAAACCCAAGGACACCCTC
ATGATCTCACGCACCCCCGAGGTCACATGCGTGGTGGTGGACGTGAGCCAGGATGACCCCGAGGT
GCAGTTCACATGGTACATAAACAACGAGCAGGTGCGCACCGCCCGGCCGCCGCTACGGGAGCAGC
AGTTCAACAGCACGATCCGCGTGGTCAGCACCCTCCCCATCGCGCACCAGGACTGGCTGAGGGGC
AAGGAGTTCAAGTGCAAAGTCCACAACAAGGCACTCCCGGCCCCCATCGAGAAAACCATCTCCAA
AGCCAGAGGGCAGCCCCTGGAGCCGAAGGTCTACACCATGGGCCCTCCCCGGGAGGAGCTGAGCA
GCAGGTCGGTCAGCCTGACCTGCATGATCAACGGCTTCTACCCTTCCGACATCTCGGTGGAGTGG
GAGAAGAACGGGAAGGCAGAGGACAACTACAAGACCACGCCGGCCGTGCTGGACAGCGACGGCTC
CTACTTCCTCTACAGCAAGCTCTCAGTGCCCACGAGTGAGTGGCAGCGGGGCGACGTCTTCACCT
GCTCCGTGATGCACGAGGCCTTGCACAACCACTACACGCAGAAGTCCATCTCCCGCTCTCCGGG
TAAATGA
(SEQ ID NO: 63)
GCGCTGTGCCGGCGATATC (SEQ ID NO: 69) (pcDNA3.4) . . .
                                    EcoRV
Translated H-Chain with CDRs Marked
```

| | |
|---|---|
| METGLRWLLLVAVLKGVLCQSLEESGGDLVKPGASLILTCKGSGFSFTIR | 50 |
| YNICWVRQAPGKGLEWIACVNSAYASWAKGRFTISKTSSTTVTLQMTSLT | 100 |
| AADTATYFCVRYVDSRYYGVDLWGPGTLVTVSSGQPKAPSVFPLAPCCGD | 150 |
| TPSSTVTLGCLVKGYLPEPVTVTWNSGTLINGVRTFPSVRQSSGLYSLSS | 200 |
| VVSVTSSSQPVTCNVAHPATNTKVDKTVAPSTCSKPMCPPPELLGGPSVF | 250 |
| IFPPKPKDTLMISRTPEVTCVVVDVSQDDPEVQFTWYINNEQVRTARPPL | 300 |
| REQQFNSTIRVVSTLPIAHQDWLRGKEFKCKVHNKALPAPIEKTISKARG | 350 |
| QPLEPKVYTMGPPREELSSRSVSLTCMINGFYPSDISVEWEKNGKAEDNY | 400 |
| KTTPAVLDSDGSYFLYSKLSVPTSEWQRGDVFTCSVMHEALHNHYTQKSI | 450 |
| SRSPGK* | |

```
(SEQ ID NO: 61)
>Clone pEB1934P-4A5-K1 in pcDNA3.4
. . . (pcDNA3.4) TCCGGACTCTAGCACC (SEQ ID NO: 68)
                            BspEI XbaI (partial)
ATGGACACGAGGGCCCCCACTCAGCTCCTGGGGCTCCTGCTGCTCTGGCTCCCAGGTGCCACATT
TGCCCAAGTGCTGACCCAGACTCCATCGTCCGTGTCTGCAGCTCTGGGAGGCACAGTCACCATCA
GTTGCCAGTCCAGTGAGAGTGTTTATAAGAACAACTACTTATCCTGGTATCAACAGAAACCAGGG
CAGCCTCCCAAGCTCCTGATCTATGGTGCATCCACTCTGGCATCTGGGGTCCCATCGCGGTTCAA
AGGCAGTGGATCTGGGACACAGTTCACTCTCACCATCAGCAGCGTGCAGTGTGACGATGCTGCCA
CTTACTACTGTCAAGGCGGTTATAGGGGCGATTATAGTAGTGGTGATGGTATTCTTTTCGGCGGA
GGGACCGAGGTGGTCGTCAAAGGTGATCCAGTTGCACCTACTGTCCTCATCTTCCCACCATCTGC
TGATCTTGTGGCAACTGGAACAGTCACCATCGTGTGTGTGGCGAATAAATACTTTCCCGATGTCA
CCGTCACCTGGGAGGTGGATGGCACCACCCAAACAACTGGCATCGAGAACAGTAAAACACCGCAG
AATTCTGCAGATTGTACCTACAACCTCAGCAGCACTCTGACACTGACCAGCACACAGTACAACAG
CCACAAAGAGTACACCTGCAAGGTGACCCAGGGCACGACCTCAGTCGTCCAGAGCTTCAATAGGG
GTGACTGTTAG
(SEQ ID NO: 64)
AGCGATATC (SEQ ID NO: 70) (pcDNA3.4) . . .
                EcoRV
Translated K-Chain with CDRs Marked
```

| | |
|---|---|
| MDTRAPTQLLGLLLLWLPGATFAQVLTQTPSSVSAALGGTVTISCQSSES | 50 |
| VYKNNYLSWYQQKPGQPPKLLIYGASTLASGVPSRFKGSGSGTQFTLTIS | 100 |
| SVQCDDAATYYCQGGYRGDYSSGDGILFGGGTEVVVKGDPVAPTVLIFPP | 150 |
| SADLVATGTVTIVCVANKYFPDVTVTWEVDGTTQTTGIENSKTPQNSADC | 200 |
| TYNLSSTLTLTSTQYNSHKEYTCKVTQGTTSVVQSFNRGDC* | |

(SEQ ID NO: 62)

### Statistics

Statistical analysis was performed using Prism 8.4.2 (GraphPad Software). Student's t-test was used to determine the significant difference between the two groups. Experimental replicates or sample sizes presented as n are provided in the figure legends. Differences between the groups were considered significant at p<0.05. The degrees of significance were reported as *p<0.05, **p<0.01, ***p<0.001.

### Results

### CGRP Promoter Driven Aberrant Cdk5 Develops Aggressive Tumors

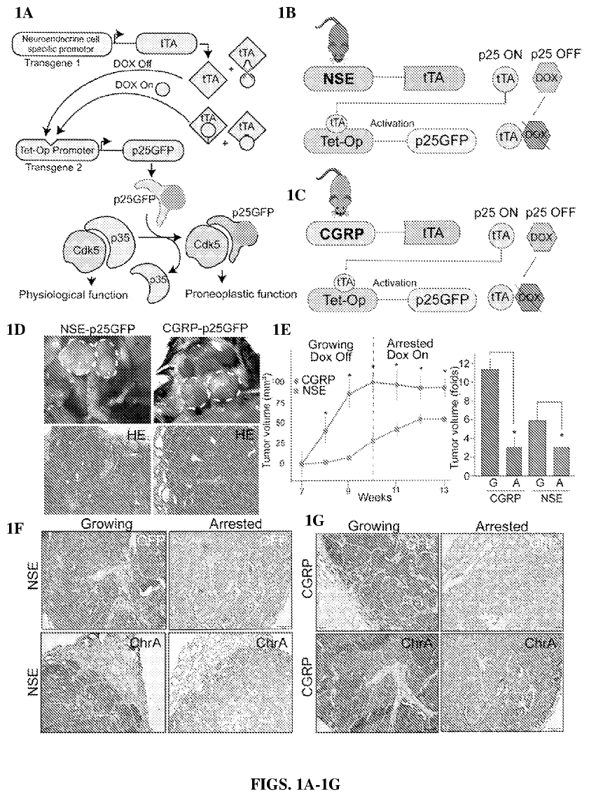

While Cdk5 is widely implicated in brain disorders, its tumorigenic function is still nascent. To gain insights into the functional role of Cdk5 in tumor progression at the genomic and transcriptomic level, bi-transgenic mouse tumor models were engineered where tumorigenesis is induced by aberrant Cdk5 activation. A doxycycline-regulated system was deployed where NE cell-specific promoter-driven tetracycline transactivator (tTA) induces p25 overexpression (p25OE) resulting in tumor development at the orthotopic site. Eventually, p25OE triggers reciprocity of Cdk5 with p25 over p35 rendering aberrant kinase activity. The resultant Cdk5/p25 interaction facilitates pro-neoplastic signaling compared to its physiological counterpart, i.e. Cdk5/p35 (FIG. 1A).

p25OE in calcitonin-secreting C cells is controlled by the neuron-specific enolase (NSE) promoter developed bilateral MTCs in mice [10](FIG. 1B). Calcitonin gene-related peptide (CGRP) is a splice variant of the calcitonin gene which translates into a neuropeptide localized in neuronal and neuroendocrine cells. Studies have shown that CGRP promoters are more efficient in restricting the transgene expression in calcitonin-secreting C cells in comparison to neuronal cells [25, 26]. Hence, the CGRP promoter was exploited in this transgenic system to induce stringent p25OE in thyroid C cells while preventing leaky expression in off-target tissues (FIG. 1C). Indeed, both NSE and CGRP promoter-driven p25OE developed MTC tumors (FIG. 1D). However, the tumor onset and growth rate of CGRP-p25OE model was significantly higher than that of NSE-p25OE. The tumor size in CGRP was ~100 mm³ vs. 25 mm³ in NSE at 10 weeks after doxycycline removal, showing a ~4-fold tumor growth rate increase in CGRP-p25OE mice (FIG. 1E). Both murine models overexpressed p25GFP and chromogranin A (Chr A) in growing tumors signify typical NETs features (FIG. 1F-1G). Of note, p25GFP expression was drastically decreased in arrested conditions confirming the dependency of tumor growth on Cdk5 (FIG. 1F-1G). The comparable p25GFP expression between NSE and CGRP growing tumors indicates the involvement of other factors causing rapid tumor growth in CGRP over the NSE model. The data show that CGRP-p25OE develops early-onset aggressive forms of MTCs compared to the previously reported NSE-p25OE mice. These results prompted examination of the downstream nodes of Cdk5 signaling in these models to identify plausible effectors associated with rapid tumor growth.

### Genomic Alteration Landscape in Mouse and Human Tumors

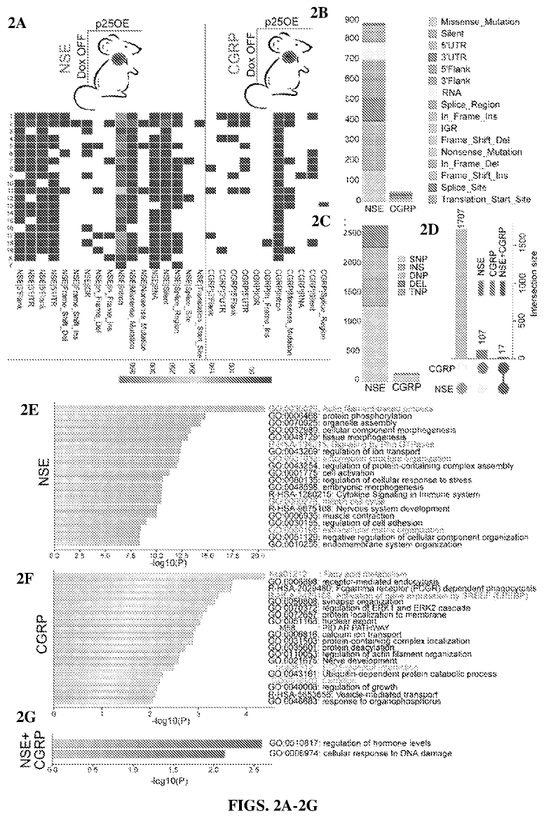

Whole-exome sequencing (WES) was conducted to determine the mutational profile of NSE and CGRP MTC models. Both models displayed highly heterogeneous mutational profiles across the chromosomes (FIG. 2A) where NSE-p25OE harbored a higher average number of somatic mutations ~876 compared to ~50 in CGRP-p25OE (FIG. 2B). Of these, the majority include 154 missense, 176 silent, and 66 5′UTR mutations in NSE-p25OE. In contrast, CGRP-p25OE accumulated 12 missense, 9 silent, and 6 5′UTR (FIG. 2B). Furthermore, exome data displayed drastic differences in the number of SNPs, insertions, and deletions between NSE and CGRP tumors, likely causing frameshifts (FIG. 2C). The number of mutated genes unique to NSE tumors were 1707, while CGRP tumors harbored mutations in 107 unique genes. Also, 17 mutated genes were common between the two models (FIG. 2D). Of note, not all SNPs are associated with cancer progression and their impact may vary depending on the location of the SNPs within the genome. The high frequency of genetic variation including silent SNPs may not have a direct impact on tumor progression of NSE but can still indicate crucial information about the genetic landscape of tumors. The direct effect of genetic variations identified in NSE/CGRP tumors is not yet fully understood and requires further investigation.

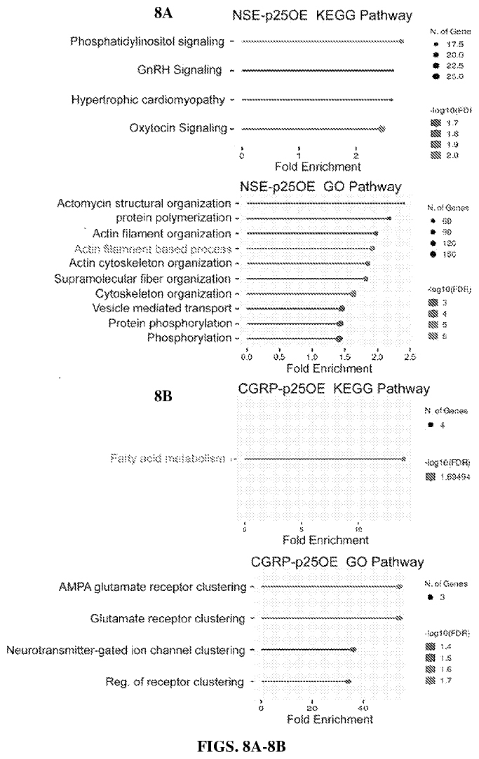

To understand the molecular processes underlying tumorigenesis of NSE/CGRP models, pathway enrichment analyses of altered genes were performed. The most highly enriched mutated gene set in NSE-p25OE tumors was involved in ‘actin filament-based process’ (FIG. 2E). Other altered cancer-related GO pathways included mitotic cell cycle, Rho-GTPases, cytokine signaling, cell adhesion, and extracellular matrix organization (FIG. 2E). In addition, KEGG database indicated enrichment of phosphatidylinositol, GnRH (gonadotrophin receptor hormone), and oxytocin signaling including actin/cytoskeletal-based processes (FIG. 8A). Dysregulation in the GnRH and oxytocin signaling pathways have been implicated in several cancers [27, 28], but unexplored in MTC, indicating an important avenue for further investigation. In contrast, KEGG enrichment of mutated genes in CGRP-p25OE tumors displayed ‘Fatty acid metabolism’ as the most significantly altered pathway besides glutamate receptor clustering (FIGS. 2F and 8B). Of note, glutamate signaling is actively involved in bioenergetics and metabolic pathways in cancer [29]. In agreement, glutamate receptor antagonists are known to suppress MTC and carcinoid NET growth and metabolic activity [30]. Likewise, the activation of SREBP, a master transcription factor, and regulator of lipid metabolism [31] was altered in CGRP mice (FIG. 2F). These data provide evidence of metabolic derangement in CGRP-p25OE mice. The overlapping mutated genes between NSE and CGRP were prominently enriched in the ‘regulation of hormone’, a characteristic of MTC patients showing high biosynthetic and hormone secretory activity [32]. In addition, ‘cellular response to DNA damage’ processes were identified in both NSE/CGRP models (FIG. 2G). Based on previous reports, activation of DNA damage response genes is common in MTC whereby modification of chromatin machinery favors a drug-resistant phenotype [33, 34].

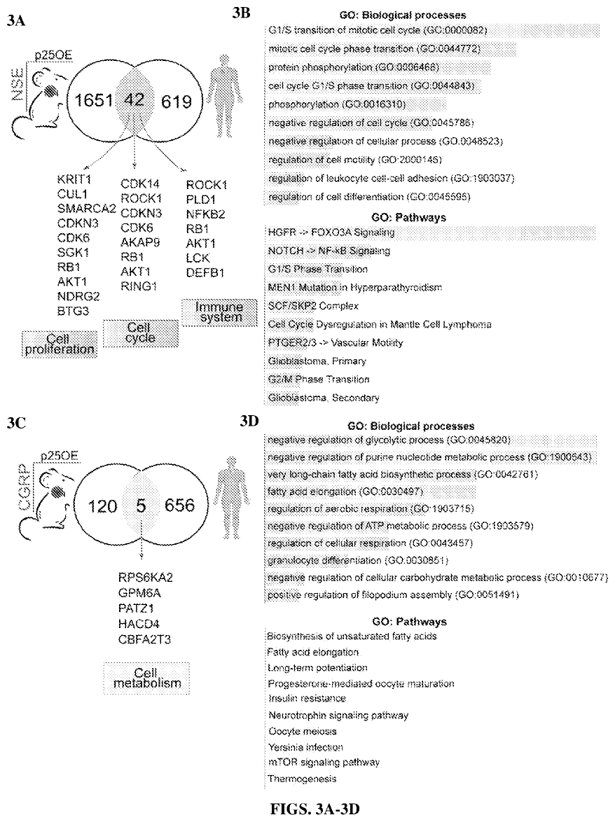

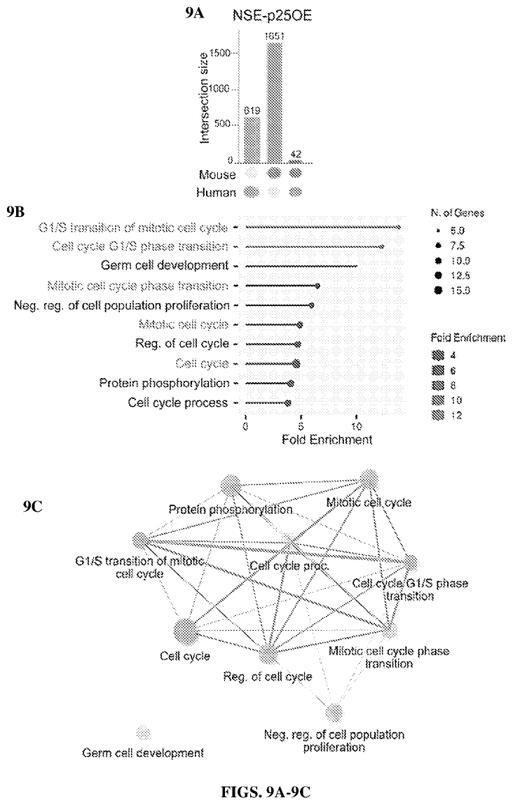

Heretofore, it was shown that activation of aberrant Cdk5 develops human-like MTC that accumulates mutations altering cell cycle and metabolic profiles in respective models. However, it is important to understand if the mutational landscapes of mouse tumors emulate their human counterparts. To evaluate the application of these tumor models as prototypes for human disease, mutated genes in mouse and human tumors were compared. Out of 1693 altered genes, 42 orthologs were common between NSE-p25OE and human MTC [35](FIG. 3A, 9A). The prominent genes intersecting between human and mouse MTC, such as RB1, AKT1, and NDGR2, are integral to processes including cell proliferation, cell cycle, and immune system function [36-38]. Several other common mutated genes including SMARCA2, Cdk6, BTG3, ROCK1, PLD1, and DEFB1 play important roles in cancer progression but are unexplored in MTC and represent interesting targets for future investigation (FIG. 3A). The top significantly enriched biological processes common between NSE-p25OE and human tumors include ‘G1/S transition of mitotic cycle’, and ‘mitotic cell cycle phase transition’ (FIGS. 3B and 9B-9C). Moreover, pathways linked to mutated genes were highly clustered across FOXO3A signaling, NOTCH-NFkB signaling, and G1/S phase transition, consistent with those previously reported in MTC patients (FIG. 3B) [39-41].

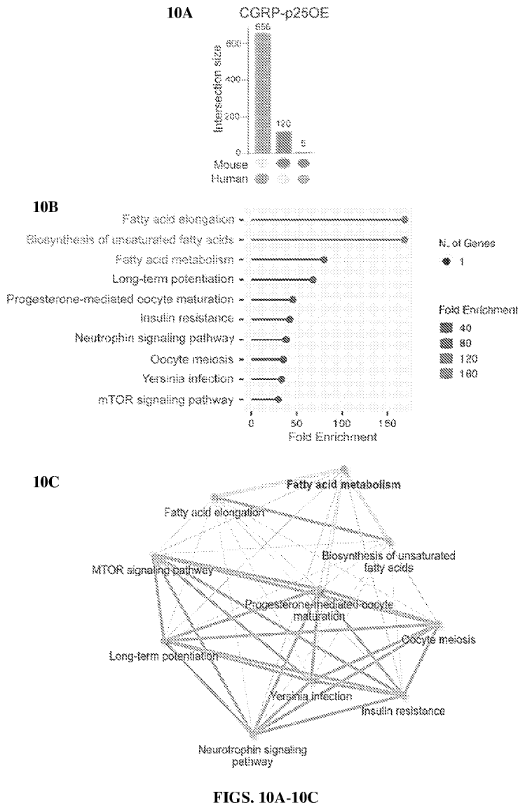

Conversely, CGRP-p25OE mouse vs. human tumor comparison revealed five intersecting genes harboring mutations in RPS6KA2, GPM6A, PATZ1, HACD4, and CBFA2T3 (FIG. 3C, 10A). Biological processes connected to altered genes were mainly involved in cellular metabolism (i.e., fatty acid metabolism, glycolytic process, and nucleotide metabolism). Notably, a significant impact on the biosynthesis of unsaturated fatty acids and fatty acid elongation pathways was recognized (FIGS. 3D and 103B-10C). In concordance, impairments in fatty acid metabolism, purine metabolism, and tri-carboxylic acid cycle were reported in MTC patients compared to healthy controls [42]. In summary, the results demonstrated a profile of signaling pathways altered in human tumors and replicated in the mouse models. Of particular interest are the ‘mitotic cell cycle process’ in NSE-p25OE and ‘metabolic impairment’ in CGRP-p25OE models. Further in-depth understanding of these processes can uncover the underlying cause of the differential rate of tumor progression in individual models and possibly human patients.

Functional Impact of p25OE on Transcriptomics of Mouse Tumors

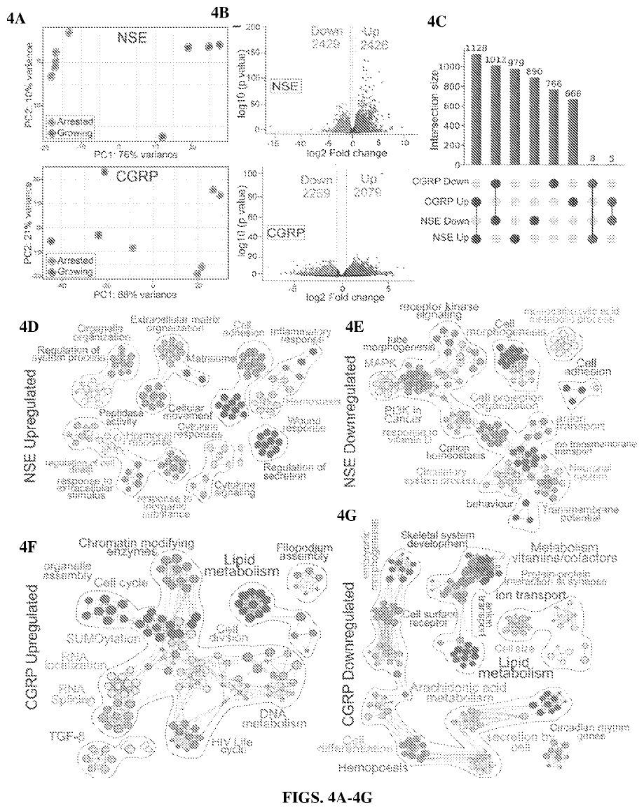

Gene mutations have the propensity to induce transcriptional changes which influence cancer cell progression and responses to chemotherapy. To identify the alterations in gene expression, bulk RNA sequencing was performed on growing and arrested MTC tumors derived from NSE and CGRP models. The principal component analysis demonstrated distinct segregation of gene expression between growing and arrested tumors derived from NSE/CGRP (FIG. 4A). Further gene expression analysis indicates a total of 4920 differentially expressed genes (DEGs), of which 2426 were upregulated and 2429 were downregulated in growing NSE-p25OE tumors. Whereas CGRP-p25OE revealed a total of 4348 DEGs, of which 2079 were upregulated and 2269 were down-regulated (FIG. 4B). Intersection size of unique and common DEGs up- and down-regulated in NSE and CGRP tumors was determined by upset plots. Notably, both NSE/CGRP tumors showed upregulated transcripts of Cdk5R1 and Cdk5RAP2 indicating augmented transcriptional regulation of Cdk5 signaling components in these models (FIG. 4C).

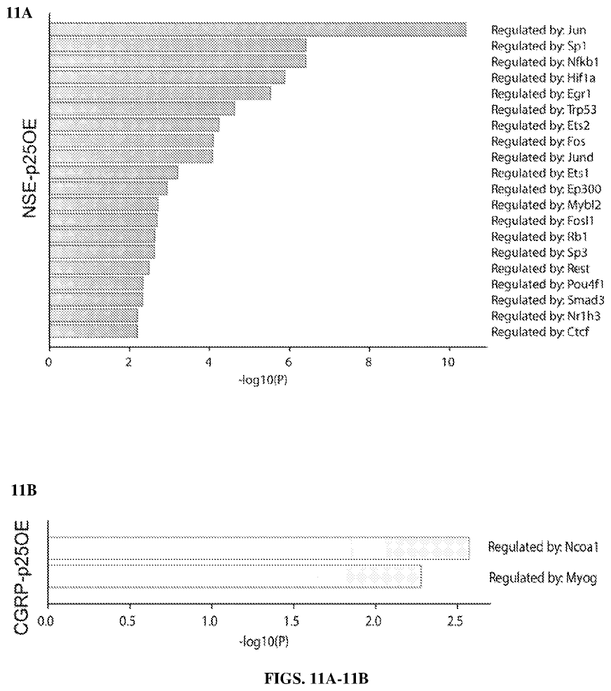

Pathway and process analyses performed on DEGs were clustered based on similarities of enriched terms (p-value<0.01, gene count, and enrichment factor>1.5). Network analysis of enriched nodes with a similarity score of >0.3 was connected by edges. Accordingly, upregulated DEGs in NSE tumors show hallmark clusters involved in processes such as cell adhesion, positive regulation of cell migration, cell motility, actin filament polymerization, cytokine signaling, T-cell activation, and lymphocyte proliferation (FIG. 4D). The downregulated DEG in NSE tumors clustered cell morphogenesis, ion transport, receptor kinases, MAPK, and PI3K activities (FIG. 4E). Notably, processes such as tissue morphogenesis, ion transport, cytokine signaling, and extracellular matrix organization were afflicted both with mutations and transcriptional dysregulation in NSE tumors. Further, TRRUST database uncovered the transcriptional factors (TF) namely, c-Jun, Sp1, and NFkB as putative regulators of DEGs in NSE-p25GE (FIG. 11A). Interestingly, the activity of c-Jun is regulated via Rho GTPases which in turn drives the transcription of genes involved in the cell cycle progression [43]. This aligns with the exome data showing altered Rho GTPase signaling in NSE mice (FIG. 2E). Likewise, the Sp1 is a known regulator of ion transport in the thyroid cancer [44] while the enrichment of NFkB signifies a plausible contribution to cytokine and inflammatory responses in NSE-p25 tumors [45].

Analysis of upregulated DEGs in CGRP-p25OE revealed enrichment clusters across the cell cycle, cell division, metabolism of lipids, nucleotide metabolic processes, SUMOylation, RNA splicing/localization, chromatin-modifying enzyme, and transforming growth factor beta (TGFβ) (FIG. 4F). The downregulated transcripts in CGRP tumors altered many metabolic pathways like those afflicted by mutations (FIG. 4G and FIG. 2F). Also, TRRUST query identified Ncoa1 as the putative transcriptional regulator of DEGs in CGRP tumors, which is shown to regulate lipogenic and glucose metabolic pathways [46, 47](FIG. 11B). These results indicate dysregulation of distinct pathways impacted both by mutations and gene expression changes downstream of hyperactive Cdk5 in NSE/CGRP models of MTC.

### Consequence of Somatic Mutations on Gene Expression of Mouse Tumors

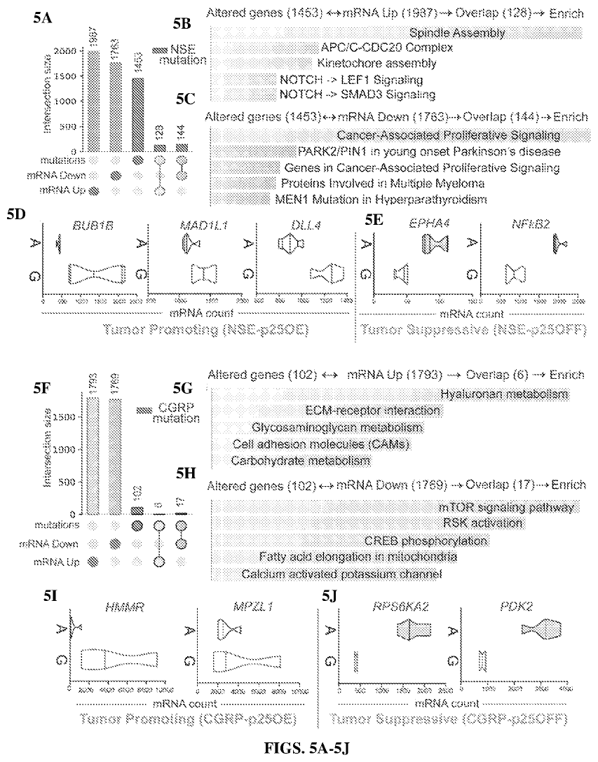

Genetic abnormalities can influence gene expression and signaling pathways contributing to the tumorigenic process. To prioritize the genes that confer a growth advantage in the MTC models, the influence of mutations on the expression level of their residing genes was determined. The upset plot shows 128 and 144 altered genes that correlate with changes in mRNA expression of NSE-p25OE tumors (FIG. 5A). The top-upregulated mRNA gene sets hit by somatic mutations were enriched in spindle assembly, APC/C-CDC20 complex, kinetochore assembly, and NOTCH signaling pathways (FIG. 5B). Conversely, the key mutated genes that correlated with downregulated transcripts were primarily involved in cancer-associated proliferative signaling (FIG. 5C). Subsequent prioritization of mutated genes based on changes in gene expression displayed BUB1B, MAD1L1, and DDL4 as main targets, which were elevated in growing tumors compared to arrested tumors signifying a tumor-promoting role (FIG. 5D). In addition, the suppression of EPHA4 and NFkB2 mRNA levels in growing tumors indicates tumor suppressive function of the residing mutations (FIG. 5E).

Next, the consequence of mutations on the expression profile of the CGRP-p25OE model was determined. Overlap of WES with RNA seq data revealed six alterations that caused mRNA upregulation (FIG. 5F). Pathways analysis disclosed metabolic processes such as hyaluronan metabolism, glycosaminoglycan metabolism, and carbohydrate metabolism in addition to extracellular matrix-receptor interaction and cell adhesion processes associated with upregulated mutated genes (FIG. 5G). Further, 17 mutated genes correlated with transcriptional repression (FIG. 5F). Enrichment analysis of intersecting genes indicates suppression of mTOR signaling, RSK activation, CREB phosphorylation, and fatty acid elongation in mitochondria (FIG. 5H). Finally, gene prioritization based on alterations in gene and transcript levels revealed HMMR, and MPZL1 as tumor promoters, whereas RPS6KA2 and PDK2 as tumor suppressors in growing CGRP tumors (FIG. 5I-5J). In conclusion, the comparative mutational and transcriptomic landscape of CGRP/NSE models revealed candidate genes serving as potential regulators of tumor progression downstream of aberrant Cdk5.

### Histodiagnostic Characterization of Cdk5 Activity in Mouse and Human Tumors

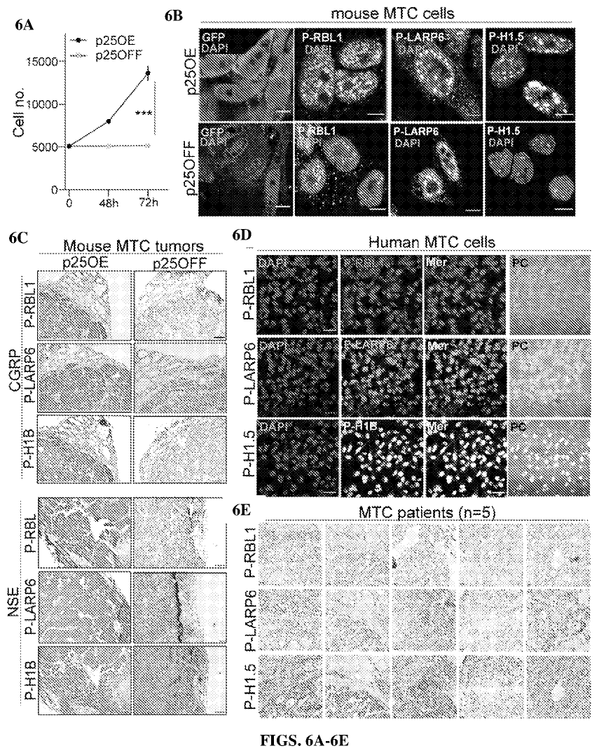

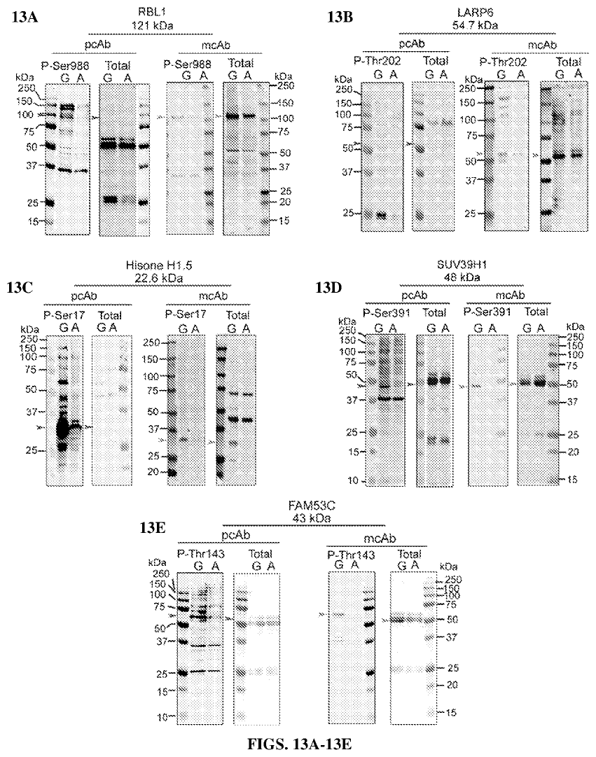

Hyperactive Cdk5 plays an important role in MTC progression [10]. A positive correlation was demonstrated between Cdk5 and its downstream targets in human MTC. The main protein phosphorylation substrates of this aberrantly active kinase included P-T143 FAM53C, P-T202 LARP6, P-S988 RBL1, P-S17 H1.5, and P-S391 SUV39H1 [48]. Both NSE/CGRP models manifest aberrant Cdk5 activation as indicated by increased Cdk5-dependent phosphorylation in growing (p25OE) versus arrested tumors (p25OFF) (FIG. S5A), indicating these phosphosites may serve as biomarkers for the detection of Cdk5-driven human tumors. However, the polyclonal antibodies first used to detect these Cdk5 targets were raised against short phospho-peptide epitopes that were limited in specificity and often detected cross-reactive proteins harboring similar phosphorylation site motifs. Considering the potential importance of aberrant Cdk5 in MTC diagnosis, more selective and specific monoclonal antibodies (mcAb) that could precisely probe Cdk5 activity in mouse and human tumors were desirable. To solve this problem, recombinant phosphorylation state-specific mcAb were generated. The specificity of mcAb in growing (G) and arrested (A) tumors were evaluated for their detection of Cdk5 phosphorylation sites by immunoblotting, immuno-cyto, and -histochemistry. Expression analysis of P-RBL1, P-LARP6, P-H1.5, P-SUV39H1, and P-FAM53C showed improved efficiency and reduced non-specific binding of mcAb over polyclonal (pcAb) (FIG. 13A-13E). Further, the mcAb was tested on mouse cells derived from NSE-p25OE cells. These cells preserve the characteristics of mouse MTC where cell proliferation is regulated by p25OE (FIG. 6A). Increased nuclear staining of Cdk5 target sites—P-RBL1, P-LARP6, and P-H1.5 were identified in growing/p25OE mouse cells compared to their arrested/p25OFF counterparts (FIG. 6B). A similar effect was mirrored in CGRP/NSE-derived mouse tumor tissues where the expression of phospho-targets was higher in p25OE vs p25OFF tumors, indicating dependency of these phosphosites on aberrant Cdk5 activity (FIG. 6C).

The improved detection of aberrant Cdk5 activity in inducible/arrestable mice models spur the efforts to evaluate these mcAbs in human cells and tissues. Human MTC TT cells known for Cdk5-dependent cell growth were utilized was probing key phosphoproteins [49]. The main phosphoproteins including, RBL1 (RB transcriptional corepressor like 1) regulate the cell cycle, and proliferation by modulating chromatin structure. LARP6 (La Ribonucleoprotein 6), a translational regulator shuttles between cytoplasm and nucleus promotes nucleic acid binding while linker histone H1.5 facilitates chromatin compaction. The immunocytochemical staining of TT cells displayed nuclear localization of these phosphoproteins similar to that observed in mouse MTC cells, consistent with the known function of these proteins in chromatin structure modulation. Having established selective detection of these aberrant Cdk5 effectors in human cells, histological tumor sections from a small cohort of MTC patients were obtained to assess the presence of these sites. Interestingly, the immunohistochemical results showed varying degrees of phospho-site staining across MTC patients confirming a characteristic heterogeneous expression profile of MTC tumors (FIG. 6E).

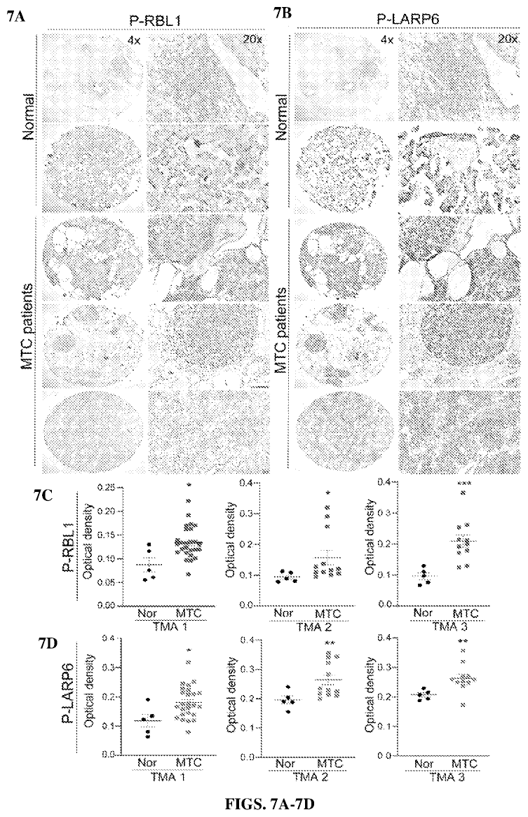

Data indicates that these phosphorylation biomarker detection reagents have the potential to identify aberrant Cdk5-driven human patient tumors. To explore this further, the expression of key Cdk5 phosphorylation sites including P-RBL1 and P-LARP6 was more broadly assessed across the three independent human MTC tissue microarrays (TMA 1-3), and compared with the normal non-tumor controls such as the prostate, placenta, spleen, and liver (FIG. 7A-7B). Quantification of phospho-site expression indicated by optical density (OD), shows 27% and 44% of patients with elevated P-RBL1 and P-LARP6 in TMA1 (stage: pT1bNX, pT2 N0) while 28% exhibited an increase in both P-LARP6/P-RBL1 (n=25; TMA1). In TMA 2, 25% of patients with lymph node metastasis showed increased P-RBL1/P-LARP6 (n=12; stage: pT3N1b). Quantification of TMA 3 displayed an increase of both phosphosites in ~36% of patients (n=11; stage: pT2NX and few cases with metastatic deposits in lymph nodes) (FIG. 7C-7D). Overall histo-analysis of tissue microarrays showed a significant increase of Cdk5 phosphosites in patient tumors trending towards metastasis compared to the normal tissues (FIG. 7A-7D). Until last year, the World Health Organization (WHO) did not recommend the testing of Ki-67 proliferation index in cases of MTC. Therefore, it is advisable to acknowledge the fact that the dataset cases were not tested for Ki-67 as it was not considered a standard practice during the time of surgery and pathological diagnosis. In summary, these findings characterize the efficacy of mcAbs in identifying aberrant Cdk5 activity across mouse and human tumors manifesting diagnostic value in stratifying cancer patients that are most likely to have clinical benefits from Cdk5 inhibitors.

### SUMMARY

The lack of reliable animal models that mimics aggressive MTC disease is a major setback in the investigation of genetic alterations, identification of diagnostic biomarkers, and molecular mechanisms associated with malignant growth. Cdk5 is aberrantly activated in several NETs including MTC, contributing to tumor development. To circumvent the lack of appropriate models, Cdk5 was used as a tool to generate conditional transgenic mice that develops ‘slow’ and ‘rapid’ onset human-like MTC tumors. Aberrant Cdk5 can be characterized by the simultaneous activation of multiple pathways regulating cell proliferation and invasion. Hence it is important to capture the complexity of signal transduction downstream of hyperactive Cdk5 to identify distinctive markers of aggressiveness and targets for therapeutic intervention. Here two mouse models of MTC are characterized, namely NSE-p25OE and CGRP-p25OE, mediating mild and aggressive onset of the disease. To understand the genes and pathway perturbation in these models, exomic and RNA sequencing was performed in tumors derived from the respective models. Computational analysis identified key pathways associated with the altered genes in mouse tumors and those intersecting with human tumors. In addition, important genes were prioritized based on changes in the gene and transcript levels.

Major pathways associated with altered genes in NSE-p25OE tumors were involved in actin filament-based processes. Actin-dependent enrichment included mitotic cell cycle, cell adhesion, Rho GTPase, actomyosin, and extracellular matrix organization, processes known for their role in cancer cell proliferation [50-53]. In agreement, the causal impact of unbalanced actin dynamics in MTC tumor invasiveness and growth has been mentioned [54]. Cdk5 is known to regulate actin microtubule cytoskeleton, indicating a Cdk5-dependent phenotype is acquired by the NSE model [55, 56]. Furthermore, the main overlapping mutated genes and pathways altered in NSE-p25OE and human tumors included RB1, AKT1, SMARCA2, PLD1, FOXO3a, Notch, NFkB, and G1/S phase transition. Notably, many of these pathways have previously demonstrated oncogenic or tumor-suppressive roles in thyroid cancer [36, 39, 40, 57]. Moreover, network pathway analysis of transcriptomic data revealed recurrent pathways afflicted both by mutations and gene expression changes. These data indicate that neoplastic transformation in the slow growing NSE-p25OE model recapitulates the indolent form of human disease that has a higher plausibility of dysregulated actin dynamics and mitotic cell cycle processes.

Conversely, the rapid growing CGRP-p25OE model harbored mutations that predominantly impacted fatty acid metabolism. This finding is supported by a recent study that demonstrated perturbation of fatty acid metabolism in MTC patients compared to healthy controls [42]. In addition, the overlapping mutated genes between CGRP-p25OE mouse and human tumors clustered pathways associated with fatty acid biosynthesis. This highlights the likelihood of metabolic dysregulations in instilling aggressive phenotype in CGRP mouse and human MTCs. An increasing number of studies show that malignant tumors are highly dependent on lipid metabolism and fatty acid synthesis for growth and survival [58, 59]. Cdk5 is an emerging candidate entangled in several metabolic conditions including cancer, diabetes, and obesity [60][61]. Cdk5-dependent phosphorylation of PPARγ and PRKAG2 impairs key metabolic sensors such as adipsin, adiponectin, and AMPK kinase [12, 62]. A direct connection between Cdk5 and lipid metabolism was recently reported where Cdk5-mediated phosphorylation of acetyl-CoA synthetase 2 (ACSS2) was shown to promote glioblastoma growth by regulating lipid production [63].

Following WES, transcriptomic analysis of CGRP-p25OE tumors also revealed alteration in metabolic pathways including lipid metabolism, arachidonic acid metabolism, DNA metabolism, and metabolism of vitamins/cofactors. Apart from metabolism, cell cycle, and cell division mRNA clusters were enriched in CGRP tumors. Periodic expression of cell cycle regulatory genes facilitates tumor cell proliferation [64]. Accordingly, altered expression of cell cycle regulatory genes such as PTTG1, AURKA, or loss of CDK/RB, p18, and p27 are known for promoting aggressiveness in MTC [65, 66]. It is believed that dysregulation of lipid metabolism and cell cycle processes in orchestration with aberrant Cdk5 contributes to the aggressive phenotype in CGRP-p25OE tumors.

The transcriptional correlates of mutated genes were also examined to prioritize important genes in mice models. The principal mutations that induced changes in the mRNA expression of NSE-p25OE were in the leading edge of mitotic spindle assembly and Notch signaling components. The findings, in concordance with previous studies, indicate that abnormal mitotic spindle and chromosomal instability can be vital drivers in MTC progression [67][68]. The involvement of Notch in the development of thyroid C-cells and MTC growth further corroborate our analysis [40]. The spindle assembly and Notch-related genes including BUB1B, MAD1L1, and DLL4, showed increased expression in growing tumors and, hence, may be viewed as tumor-promoters, whereas downregulated expression of mutated EPHA4 and NFKB2 in growing tumors as tumor suppressors.

Assessment of mutational impact on gene expression of CGRP-p25OE mouse revealed perturbation in metabolic and mTOR signaling pathways. In fact, the disruption of metabolic pathways was consistent across the exome-sequencing and transcriptomic data, indicating the need for functional investigation of metabolic targets in aggressive tumors. Expression of the mutated gene HMMR was highly elevated in growing CGRP-p25 tumors. HMMR (hyaluronan-mediated motility receptor) is a putative neoplastic marker and a functional component of metabolic pathways associated with cancer progression and poor clinical outcomes [69]. Moreover, mutations and concomitantly decreased expression of RPS6KA2 (Ribosomal protein S6 kinase A2) and PDK2 (Pyruvate dehydrogenase kinase 2) implicate these two genes as potential tumor repressors in CGRP tumors. Both RPS6KA2 and PDK2 are components of PI3K/Akt/mTOR pathway involved in the regulation of cell cycle progression and metabolic sensing in glycolytic cancers [70][71][72].

Alterations at the genetic and transcriptional levels of certain genes within a pathway can influence other genes eliciting interactions of multiple pathways. The findings reinforce the need to classify patients based on aberrant Cdk5 activity, and further subclassify them into mild or aggressive forms based on the activation of distinct molecular pathways as described here.

Biomarker detection agents in the form of recombinant monoclonal antibodies that can distinguish Cdk5-dependent tumors were also developed and tested. These biomarkers can predict anti-Cdk5 therapy responsiveness in patient-derived xenografts [48].

In summary, transgenic mouse models that recapitulate both slow and rapid-onset human-like MTC tumors driven by aberrant Cdk5 activity were established. The CGRP-p25OE model mimics aggressive tumors primarily characterized by alterations in metabolic pathways. NSE-p25OE mice develop slow growing tumors characterized by dysregulation of mitotic spindle assembly and Notch signaling components. Findings encourage the development of personalized therapeutic strategies involving Cdk5 inhibitors in combination with patient-specific signaling modulators such as those targeting fatty acid metabolism or cell cycle regulators.

### REFERENCES

1. Modigliani, E., et al., Prognostic factors for survival and for biochemical cure in medullary thyroid carcinoma: results in 899 patients. The GETC Study Group. Groupe d'etude des tumeurs a calcitonine. Clin Endocrinol (Oxf), 1998. 48(3): p. 265-73.
2. Bartz-Kurycki, M. A., O. E. Oluwo, and L. F. Morris-Wiseman, Medullary thyroid carcinoma: recent advances in identification, treatment, and prognosis. Ther Adv Endocrinol Metab, 2021. 12: p. 20420188211049611.
3. Maxwell, J. E., et al., Medical management of metastatic medullary thyroid cancer. Cancer, 2014. 120(21): p. 3287-301.
4. Sippel, R. S., M. Kunnimalaiyaan, and H. Chen, Current management of medullary thyroid cancer. Oncologist, 2008. 13(5): p. 539-47.
5. Chen, H., et al., The North American Neuroendocrine Tumor Society consensus guideline for the diagnosis and management of neuroendocrine tumors: pheochromocytoma, paraganglioma, and medullary thyroid cancer. Pancreas, 2010. 39(6): p. 775-83.
6. Pajak, C., et al., (68)Ga-DOTATATE-PET shows promise for diagnosis of recurrent or persistent medullary thyroid cancer: A systematic review. Am J Surg, 2022. 224(2): p. 670-675.
7. Chen, H., et al., Effective long-term palliation of symptomatic, incurable metastatic medullary thyroid cancer by operative resection. Ann Surg, 1998. 227(6): p. 887-95.
8. Oczko-Wojciechowska, M., et al., Current status of the prognostic molecular markers in medullary thyroid carcinoma. Endocr Connect, 2020. 9(12): p. R251-R263.
9. Pozo, K. and J. A. Bibb, The Emerging Role of Cdk5 in Cancer. Trends Cancer, 2016. 2(10): p. 606-618.
10. Pozo, K., et al., The role of Cdk5 in neuroendocrine thyroid cancer. Cancer Cell, 2013. 24(4): p. 499-511.
11. Carter, A. M., et al., Cdk5 drives formation of heterogeneous pancreatic neuroendocrine tumors. Oncogenesis, 2021. 10(12): p. 83.
12. Gupta, P., et al., Genetic impairment of succinate metabolism disrupts bioenergetic sensing in adrenal neuroendocrine cancer. Cell Rep, 2022. 40(7): p. 111218.
13. Meyer, D. A., et al., Striatal dysregulation of Cdk5 alters locomotor responses to cocaine, motor learning, and dendritic morphology. Proc Natl Acad Sci USA, 2008. 105(47): p. 18561-6.
14. Lange, S., et al., Analysis pipelines for cancer genome sequencing in mice. Nat Protoc, 2020. 15(2): p. 266-315.
15. Bolger, A. M., M. Lohse, and B. Usadel, Trimmomatic: a flexible trimmer for Illumina sequence data. Bioinformatics, 2014. 30(15): p. 2114-20.
16. Li, H. and R. Durbin, Fast and accurate short read alignment with Burrows-Wheeler transform. Bioinformatics, 2009. 25(14): p. 1754-60.
17. McKenna, A., et al., The Genome Analysis Toolkit: a MapReduce framework for analyzing next-generation DNA sequencing data. Genome Res, 2010. 20(9): p. 1297-303.
18. Benjamin, D., et al., Calling Somatic SNVs and Indels with Mutect2. 2019, bioRxiv.
19. Kuilman, T., et al., CopywriteR: DNA copy number detection from off-target sequence data. Genome Biol, 2015. 16: p. 49.
20. Chen, S., et al., fastp: an ultra-fast all-in-one FASTQ preprocessor. Bioinformatics, 2018. 34(17): p. i884-i890.
21. Dobin, A., et al., STAR: ultrafast universal RNA-seq aligner. Bioinformatics, 2013. 29(1): p. 15-21.
22. Anders, S., P. T. Pyl, and W. Huber, HTSeq—a Python framework to work with high-throughput sequencing data. Bioinformatics, 2015. 31(2): p. 166-9.
23. Love, M. I., W. Huber, and S. Anders, Moderated estimation of fold change and dispersion for RNA-seq data with DESeq2. Genome Biol, 2014. 15(12): p. 550.
24. Zhuang, Y., et al., The novel function of tumor protein D54 in regulating pyruvate dehydrogenase and metformin cytotoxicity in breast cancer. Cancer Metab, 2019. 7: p. 1.
25. Baetscher, M., et al., SV40 T antigen transforms calcitonin cells of the thyroid but not CGRP-containing neurons in transgenic mice. Oncogene, 1991. 6(7): p. 1133-8.
26. Johnston, D., D. Hatzis, and M. E. Sunday, Expression of v-Ha-ras driven by the calcitonin/calcitonin gene-related peptide promoter: a novel transgenic murine model for medullary thyroid carcinoma. Oncogene, 1998. 16(2): p. 167-77.
27. Grundker, C. and G. Emons, The Role of Gonadotropin-Releasing Hormone in Cancer Cell Proliferation and Metastasis. Front Endocrinol (Lausanne), 2017. 8: p. 187.
28. Liu, H., et al., The oxytocin receptor signalling system and breast cancer: a critical review. Oncogene, 2020. 39(37): p. 5917-5932.
29. Stepulak, A., et al., Glutamate and its receptors in cancer. J Neural Transm (Vienna), 2014. 121(8): p. 933-44.
30. Haas, H. S., et al., The influence of glutamate receptors on proliferation and metabolic cell activity of neuroendocrine tumors. Anticancer Res, 2013. 33(4): p. 1267-72.
31. Wen, Y. A., et al., Downregulation of SREBP inhibits tumor growth and initiation by altering cellular metabolism in colon cancer. Cell Death Dis, 2018. 9(3): p. 265.
32. Wong, A., K. Nabata, and S. M. Wiseman, Medullary thyroid carcinoma: a narrative historical review. Expert Rev Anticancer Ther, 2022. 22(8): p. 823-834.
33. Minna, E., et al., Medullary Thyroid Carcinoma Mutational Spectrum Update and Signaling-Type Inference by Transcriptional Profiles: Literature Meta-Analysis and Study of Tumor Samples. Cancers (Basel), 2022. 14(8).
34. Cakir, M. and A. B. Grossman, Medullary thyroid cancer: molecular biology and novel molecular therapies. Neuroendocrinology, 2009. 90(4): p. 323-48.
35. Qu, N., et al., Genomic and Transcriptomic Characterization of Sporadic Medullary Thyroid Carcinoma. Thyroid, 2020. 30(7): p. 1025-1036.
36. Cerrato, A., V. De Falco, and M. Santoro, Molecular genetics of medullary thyroid carcinoma: the quest for novel therapeutic targets. J Mol Endocrinol, 2009. 43(4): p. 143-55.
37. Manfredi, G. I., et al., PI3K/Akt/mTOR signaling in medullary thyroid cancer: a promising molecular target for cancer therapy. Endocrine, 2015. 48(2): p. 363-70.
38. Yin, A., et al., Overexpression of NDRG2 Increases Iodine Uptake and Inhibits Thyroid Carcinoma Cell Growth In Situ and In Vivo. Oncol Res, 2016. 23(1-2): p. 43-51.
39. Karger, S., et al., FOXO3a: a novel player in thyroid carcinogenesis? Endocr Relat Cancer, 2009. 16(1): p. 189-99.
40. Cook, M., X. M. Yu, and H. Chen, Notch in the development of thyroid C-cells and the treatment of medullary thyroid cancer. Am J Transl Res, 2010. 2(1): p. 119-25.
41. Valenciaga, A., et al., Reduced Retinoblastoma Protein Expression Is Associated with Decreased Patient Survival in Medullary Thyroid Cancer. Thyroid, 2017. 27(12): p. 1523-1533.
42. Jajin, M. G., et al., Gas chromatography-mass spectrometry-based untargeted metabolomics reveals metabolic perturbations in medullary thyroid carcinoma. Sci Rep, 2022. 12(1): p. 8397.
43. Lukey, M. J., et al., The oncogenic transcription factor c-Jun regulates glutaminase expression and sensitizes cells to glutaminase-targeted therapy. Nat Commun, 2016. 7: p. 11321.
44. Chiefari, E., et al., Increased expression of AP2 and Sp1 transcription factors in human thyroid tumors: a role in NIS expression regulation? BMC Cancer, 2002. 2: p. 35.
45. Giuliani, C., I. Bucci, and G. Napolitano, The Role of the Transcription Factor Nuclear Factor-kappa B in Thyroid Autoimmunity and Cancer. Front Endocrinol (Lausanne), 2018. 9: p. 471.
46. Puig-Oliveras, A., et al., Expression-based GWAS identifies variants, gene interactions and key regulators affecting intramuscular fatty acid content and composition in porcine meat. Sci Rep, 2016. 6: p. 31803.
47. Motamed, M., et al., Steroid receptor coactivator 1 is an integrator of glucose and NAD+/NADH homeostasis. Mol Endocrinol, 2014. 28(3): p. 395-405.
48. Carter, A. M., et al., Phosphoprotein-based biomarkers as predictors for cancer therapy. Proc Natl Acad Sci USA, 2020. 117(31): p. 18401-18411.
49. Lin, H., et al., Cdk5 regulates STAT3 activation and cell proliferation in medullary thyroid carcinoma cells. J Biol Chem, 2007. 282(5): p. 2776-84.
50. Haga, R. B. and A. J. Ridley, Rho GTPases: Regulation and roles in cancer cell biology. Small GTPases, 2016. 7(4): p. 207-221.
51. Rodriguez-Hernandez, I., et al., Rho, ROCK and actomyosin contractility in metastasis as drug targets. F1000Res, 2016. 5.
52. Heng, Y. W. and C. G. Koh, Actin cytoskeleton dynamics and the cell division cycle. Int J Biochem Cell Biol, 2010. 42(10): p. 1622-33.
53. Vasioukhin, V. and E. Fuchs, Actin dynamics and cell-cell adhesion in epithelia. Curr Opin Cell Biol, 2001. 13(1): p. 76-84.
54. Giardino, E., et al., Cofilin is a mediator of RET-promoted medullary thyroid carcinoma cell migration, invasion and proliferation. Mol Cell Endocrinol, 2019. 495: p. 110519.
55. Strock, C. J., et al., Cyclin-dependent kinase 5 activity controls cell motility and metastatic potential of prostate cancer cells. Cancer Res, 2006. 66(15): p. 7509-15.
56. Xie, Z., et al., Serine 732 phosphorylation of FAK by Cdk5 is important for microtubule organization, nuclear movement, and neuronal migration. Cell, 2003. 114(4): p. 469-82.
57. Pacifico, F. and A. Leonardi, Role of NF-kappaB in thyroid cancer. Mol Cell Endocrinol, 2010. 321(1): p. 29-35.
58. Currie, E., et al., Cellular fatty acid metabolism and cancer. Cell Metab, 2013. 18(2): p. 153-61.
59. Ferraro, G. B., et al., Fatty Acid Synthesis Is Required for Breast Cancer Brain Metastasis. Nat Cancer, 2021. 2(4): p. 414-428.
60. Do, P. A. and C. H. Lee, The Role of CDK5 in Tumours and Tumour Microenvironments. Cancers (Basel), 2020. 13(1).
61. Lowman, X. H., et al., The proapoptotic function of Noxa in human leukemia cells is regulated by the kinase Cdk5 and by glucose. Mol Cell, 2010. 40(5): p. 823-33.
62. Choi, J. H., et al., Anti-diabetic drugs inhibit obesity-linked phosphorylation of PPARgamma by Cdk5. Nature, 2010. 466(7305): p. 451-6.
63. Ciraku, L., et al., O-GlcNAc transferase regulates glioblastoma acetate metabolism via regulation of CDK5-dependent ACSS2 phosphorylation. Oncogene, 2022. 41(14): p. 2122-2136.
64. Dominguez, D., et al., A high-resolution transcriptome map of cell cycle reveals novel connections between periodic genes and cancer. Cell Res, 2016. 26(8): p. 946-62.
65. Pezzani, R., et al., Novel Prognostic Factors Associated with Cell Cycle Control in Sporadic Medullary Thyroid Cancer Patients. Int J Endocrinol, 2019. 2019: p. 9421079.
66. Valenciaga, A., et al., Transcriptional targeting of oncogene addiction in medullary thyroid cancer. JCI Insight, 2018. 3(16).
67. Yang, X., et al., TPX2 overexpression in medullary thyroid carcinoma mediates TT cell proliferation. Pathol Oncol Res, 2014. 20(3): p. 641-8.
68. Tuccilli, C., et al., Preclinical testing of selective Aurora kinase inhibitors on a medullary thyroid carcinoma-derived cell line. Endocrine, 2016. 52(2): p. 287-95.
69. Liu, M., C. Tolg, and E. Turley, Dissecting the Dual Nature of Hyaluronan in the Tumor Microenvironment. Front Immunol, 2019. 10: p. 947.
70. Slattery, M. L., et al., Genetic variation in RPS6KA1, RPS6KA2, RPS6KB1, RPS6KB2, and PDK1 and risk of colon or rectal cancer. Mutat Res, 2011. 706(1-2): p. 13-20.
71. Bignone, P. A., et al., RPS6KA2, a putative tumour suppressor gene at 6q27 in sporadic epithelial ovarian cancer. Oncogene, 2007. 26(5): p. 683-700.
72. Atas, E., M. Oberhuber, and L. Kenner, The Implications of PDK1-4 on Tumor Energy Metabolism, Aggressiveness and Therapy Resistance. Front Oncol, 2020. 10: p. 583217.
73. Han, H., et al., TRRUST: a reference database of human transcriptional regulatory interactions. Sci Rep, 2015. 5: p. 11432.
74. Zhou, Y., et al., Metascape provides a biologist-oriented resource for the analysis of systems-level datasets. Nat Commun, 2019. 10(1): p. 1523.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An antibody or other molecule comprising an antigen binding domain of an antibody that immunospecifically binds to the epitope:
(i)
(SEQ ID NO: 1)
CSIYI-pS-PHKN;
(ii)
(SEQ ID NO: 14)
CAPVEK-pS-PAK;
(iii)
(SEQ ID NO: 27)
CALA-pT-PQKNGRV;
(iv)
(SEQ ID NO: 40)
CLAGLPG-pS-PKKRVR;
or
(v)
(SEQ ID NO: 53)
CAPSKLW-pT-PIKH.

2. The antibody or other molecule of claim 1, wherein the antigen binding domain comprises six complementarity-determining regions (CDRs), wherein the CDRs comprise one, two, three, four, five, or six consensus CDRs of the CDRs of:
(i) anti-RBL1 antibody RBL1-1;
(ii) anti-H1.5 antibody H1-5-1;
(iii) anti-LARP6 antibody LARP6-1;
(iv) anti-SUV39H1 antibody SUV39H1-1; or
(v) anti-FAM53C antibody FAM53C-1.

3. The antibody or other molecule of claim 2, wherein the CDRs comprise one, two, three, four, five, or six consensus CDRs of the CDRs of:
(i) a heavy chain variable region comprising the amino acid sequence of SEQ ID NO:2 and/or a light chain variable region comprising the amino acid sequence of SEQ ID NO: 3;
(ii) a heavy chain variable region comprising the amino acid sequence of SEQ ID NO: 15 and/or a light chain variable region comprising the amino acid sequence of SEQ ID NO: 16;
(iii) a heavy chain variable region comprising the amino acid sequence of SEQ ID NO:28 and/or a light chain variable region comprising the amino acid sequence of SEQ ID NO: 29;
(iv) a heavy chain variable region comprising the amino acid sequence of SEQ ID NO:41 and/or a light chain variable region comprising the amino acid sequence of SEQ ID NO: 42; or
(v) a heavy chain variable region comprising the amino acid sequence of SEQ ID NO: 54 and/or a light chain variable region comprising the amino acid sequence of SEQ ID NO:55.

4. The antibody or other molecule of claim 2, wherein the CDRs comprise one, two, three, four, five, or six comprising the amino acid sequence(s) of:
(i) SEQ ID NOS:4-9 or a variant thereof with at least 70% sequence identity thereto;
(ii) SEQ ID NOS:17-22 or a variant thereof with at least 70% sequence identity thereto;
(iii) SEQ ID NOS:30-35 or a variant thereof with at least 70% sequence identity thereto;
(iv) SEQ ID NOS:41-48 or a variant thereof with at least 70% sequence identity thereto; or
(v) SEQ ID NOS:56-59, 34, and 60 or a variant thereof with at least 70% sequence identity thereto.

5. The antibody or other molecule of claim 4, wherein the CDRs comprise the six CDRs of:
(i) SEQ ID NOS:4-9 in the same order and orientation as presented in SEQ ID NOS:2 and 3, respectively;
(ii) SEQ ID NOS:17-22 in the same order and orientation as presented in SEQ ID NOS:15 and 16, respectively;
(iii) SEQ ID NOS:30-35 in the same order and orientation as presented in SEQ ID NOS:28 and 29, respectively;
(iv) SEQ ID NOS:41-48 in the same order and orientation as presented in SEQ ID NOS:41 and 42, respectively; or
(v) SEQ ID NOS:56-59, 34, and 60 in the same order and orientation as presented in SEQ ID NOS:54 and 55, respectively.

6. The antibody or other molecule of claim 2, comprising
(i) a heavy chain variable region comprising the amino acid sequence of SEQ ID NOS:2 or a variant thereof with at least 70% sequence identity thereto and/or a light chain variable region comprising the amino acid sequence of SEQ ID NOS:3 or a variant thereof with at least 70% sequence identity thereto;
(ii) a heavy chain variable region comprising the amino acid sequence of SEQ ID NOS:15 or a variant thereof with at least 70% sequence identity thereto and/or a light chain variable region comprising the amino acid sequence of SEQ ID NOS:16 or a variant thereof with at least 70% sequence identity thereto;
(iii) a heavy chain variable region comprising the amino acid sequence of SEQ ID NOS:28 or a variant thereof with at least 70% sequence identity thereto and/or a light chain variable region comprising the amino acid sequence of SEQ ID NOS:29 or a variant thereof with at least 70% sequence identity thereto;
(iv) a heavy chain variable region comprising the amino acid sequence of SEQ ID NOS:41 or a variant thereof with at least 70% sequence identity thereto and/or a light chain variable region comprising the amino acid sequence of SEQ ID NOS:42 or a variant thereof with at least 70% sequence identity thereto; or
(v) a heavy chain variable region comprising the amino acid sequence of SEQ ID NOS:54 or a variant thereof with at least 70% sequence identity thereto and/or a light chain variable region comprising the amino acid sequence of SEQ ID NOS:55 or a variant thereof with at least 70% sequence identity thereto.

7. The antibody or other molecule of claim 6 comprising
(i) a heavy chain variable region wherein the heavy chain variable region comprises the amino acid sequence of SEQ ID NOS:2 and a light chain variable region wherein the light chain variable region comprises the amino acid sequence of SEQ ID NOS:3 or a variant thereof with at least 70% sequence identity thereto;
(ii) a heavy chain variable region wherein the heavy chain variable region comprises the amino acid sequence of SEQ ID NOS:15 and a light chain variable region wherein the light chain variable region comprises the amino acid sequence of SEQ ID NOS:16 or a variant thereof with at least 70% sequence identity thereto;
(iii) a heavy chain variable region wherein the heavy chain variable region comprises the amino acid sequence of SEQ ID NOS:28 and a light chain variable region wherein the light chain variable region comprises the amino acid sequence of SEQ ID NOS:29 or a variant thereof with at least 70% sequence identity thereto;
(iv) a heavy chain variable region wherein the heavy chain variable region comprises the amino acid sequence of SEQ ID NOS:41 and a light chain variable region wherein the light chain variable region comprises the amino acid sequence of SEQ ID NOS:42 or a variant thereof with at least 70% sequence identity thereto; or
(v) a heavy chain variable region wherein the heavy chain variable region comprises the amino acid sequence of SEQ ID NOS:54 and a light chain variable region wherein the light chain variable region comprises the amino acid sequence of SEQ ID NOS:55 or a variant thereof with at least 70% sequence identity thereto.

8. The antibody or other molecule of claim 6 comprising
(i) a heavy chain variable region wherein the heavy chain variable region comprises the amino acid sequence of SEQ ID NOS:2 and a light chain variable region wherein the light chain variable region comprises the amino acid sequence of SEQ ID NOS:3 or a variant thereof with at least 70% sequence identity thereto, without any variation in the CDRs of SEQ ID NOS:2 and 3;
(ii) a heavy chain variable region wherein the heavy chain variable region comprises the amino acid sequence of SEQ ID NOS:15 and a light chain variable region wherein the light chain variable region comprises the amino acid sequence of SEQ ID NOS:16 or a variant thereof with at least 70% sequence identity thereto, without any variation in the CDRs of SEQ ID NOS:15 and 16;
(iii) a heavy chain variable region wherein the heavy chain variable region comprises the amino acid sequence of SEQ ID NOS:28 and a light chain variable region wherein the light chain variable region comprises the amino acid sequence of SEQ ID NOS:29 or a variant thereof with at least 70% sequence identity thereto, without any variation in the CDRs of SEQ ID NOS:28 and 29;
(iv) a heavy chain variable region wherein the heavy chain variable region comprises the amino acid sequence of SEQ ID NOS:41 and a light chain variable region wherein the light chain variable region comprises the amino acid sequence of SEQ ID NOS:42 or a variant thereof with at least 70% sequence identity thereto, without any variation in the CDRs of SEQ ID NOS:41 and 42; or
(v) a heavy chain variable region wherein the heavy chain variable region comprises the amino acid sequence of SEQ ID NOS:54 and a light chain variable region wherein the light chain variable region comprises the amino acid sequence of SEQ ID NOS:55 or a variant thereof with at least 70% sequence identity thereto, without any variation in the CDRs of SEQ ID NOS:54 and 55;

9. The antibody or other molecule of claim 2 comprising:
(i) the amino acid sequences of SEQ ID NOS:10 and/or 11, with or without the cleavable signal sequence;
(ii) the amino acid sequences of SEQ ID NOS:23 and/or 24, with or without the cleavable signal sequence;
(iii) the amino acid sequences of SEQ ID NOS:36 and/or 37, with or without the cleavable signal sequence;
(iv) the amino acid sequences of SEQ ID NOS:49 and/or 50, with or without the cleavable signal sequence; or
(v) the amino acid sequences of SEQ ID NOS:61 and/or 62, with or without the cleavable signal sequence.

10. The antibody or other molecule of claim 2 comprising an amino acid sequence encoded by the nucleic acid sequence of:
(i) SEQ ID NOS:12 and/or 13;
(ii) SEQ ID NOS:25 and/or 26;
(iii) SEQ ID NOS:38 and/or 39;
(iv) SEQ ID NOS:51 and/or 52; or
(v) SEQ ID NOS:63 and/or 64.

11. The antibody or molecule of claim 2 comprising
a heavy chain constant region optionally wherein the heavy chain constant region comprises the amino acid sequence of SEQ ID NO:65 or a variant thereof with at least 70% sequence identity to SEQ ID NO:65; and
a light chain constant region, optionally wherein the light chain constant region comprises the amino acid sequence of SEQ ID NO:66 or 67 or a variant thereof with at least 70% sequence identity to SEQ ID NO:66 or 67.

12.-14. (canceled)

15. The antibody or molecule of claim 2, wherein the molecule or antibody is an antibody.

16. The antibody or molecule of claim 15, wherein the antibody is an intact antibody or functional antibody fragment or fusion protein.

17. The antibody or molecule of claim 16, wherein the functional fragment or fusion protein is selected from Fab fragments, F(ab′)2 fragments, Fab′ fragments, Fv fragments, recombinant IgG (rIgG) fragments, single chain antibody fragments optionally single chain variable fragments (scFv), and single domain antibodies optionally selected from sdAb, sdFv, and nanobody fragments.

18. The antibody or molecule of claim 15, wherein the antibody is selected from intrabodies, peptibodies, chimeric antibodies, fully human antibodies, humanized antibodies, and heteroconjugate antibodies, and multispecific antibodies optionally selected bispecific antibodies, diabodies, triabodies, tetrabodies, tandem di-scFv, and tandem tri-scFv.

19. The antibody or molecule of claim 15 wherein the antibody is an IgM, IgE, IgA, IgD, or IgG optionally an IgG1, IgG2, IgG3, or IgG4.

20. The antibody or molecule of claim 2, wherein the molecule or antibody is detectably labeled or comprises a conjugated toxin, drug, receptor, enzyme, receptor ligand.

21. A method of detecting Cdk5 activity comprising detecting the level of phosphorylation of one or more of P-T143 FAM53C, P-T202 LARP6, P-S988 RBL1, P-S17 H1.5, and P-S391 SUV39H1 by contacting a biological sample with one or more antibodies of claim 1, and detecting binding between the antibody or antibodies and the one or more of FAM53C, LARP6, RBL1, H1.5, and SUV39H1.

22. (canceled)

23. The method of claim 21, wherein binding is detected by an immunoassay, immunohistochemistry, Western blotting, surface plasmon resonance, flow cytometry (FACS) analysis, or a biochip.

24.-28. (canceled)

29. A method of diagnosing a subject with a Cdk5-related disease or disorder comprising detecting aberrant Cdk5 according to the method of claim 21.

30.-35. (canceled)


SEQUENCE LISTING (69 sequences)

SEQ ID NO: 1 - protein, 10 aa, synthetic construct
  MOD_RES 6: note = Phosphorylated Serine
CSIYIXPHKN

SEQ ID NO: 2 - protein, 114 aa, synthetic construct
QSLEESGGRLVTPGTPLKLTCTVSGFSLSDYNVGWVRQAPGKGLEWIGIMNIGISTWYASWAKGRFTISRTSTTVDLKMTSLTTEDTATYFCARGFSRNSYDIWGPGTLVTVSS

SEQ ID NO: 3 - protein, 110 aa, synthetic construct
AVLTQTPSPVSAAVGGTVTIKCQSSQSVVKNNYLSWYQQKPGQPPKLLIYETSKLASGVPSRFKGSGSGTQFTLTISDVQCDDAATYYCAGGYSSISDTTFGGGTEVVVK

SEQ ID NO: 4 - protein, 5 aa, synthetic construct
DYNVG

SEQ ID NO: 5 - protein, 16 aa, synthetic construct
IMNIGISTWYASWAKG

SEQ ID NO: 6 - protein, 9 aa, synthetic construct
GFSRNSYDI

SEQ ID NO: 7 - protein, 13 aa, synthetic construct
QSSQSVVKNNYLS

SEQ ID NO: 8 - protein, 7 aa, synthetic construct
ETSKLAS

SEQ ID NO: 9 - protein, 11 aa, synthetic construct
AGGYSSISDTT

SEQ ID NO: 10 - protein, 456 aa, synthetic construct
METGLRWLLLVAVLKGVQCQSLEESGGRLVTPGTPLKLTCTVSGFSLSDYNVGWVRQAPGKGLEWIGIMNIGISTWYASWAKGRFTISRTSTTVDLKMTSLTTEDTATYFCARGFSRNSYDIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 11 - protein, 237 aa, synthetic construct
MDTRAPTQLLGLLLLWLPGATFAAVLTQTPSPVSAAVGGTVTIKCQSSQSVVKNNYLSWYQQKPGQPPKLLIYETSKLASGVPSRFKGSGSGTQFTLTISDVQCDDAATYYCAGGYSSISDTTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 12 - DNA, 1371 bp, synthetic construct
  source 1..1371: mol_type = other DNA
atggagactgggctgcgctggcttctcctggtcgctgtgctcaaaggtgtccagtgtcagtcgctggaggagtccgggggtcgcctggtcacgcctgggacacccctgaaactcacctgcacagtctctggattctccctcagtgactacaacgtgggctgggtccgccaggctccagggaaggggctggaatggatcggaatcatgaatattggtattagcacatggtacgcgagctgggcaaaaggccgattcaccatctccagaacctcgaccacggtggatctgaaaatgaccagtctgacaaccgaggacacggccacctatttctgtgccagagggtttagtcgtaatagttatgatatatggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 13 - DNA, 714 bp, synthetic construct
  source 1..714: mol_type = other DNA
atggacacgagggcccccactcagctgctggggctcctgctgctctggctcccaggtgccacatttgccgccgtgctgacccagactccatctcccgtgtctgcagctgtgggaggcacagtcaccatcaagtgccagtccagtcagagtgttgttaagaacaactacttatcctggtatcagcagaaaccagggcaacctcccaaactcctgatctacgaaacatccaaactggcatctggggtcccatcgcggttcaaaggcagtggatctgggacacagttcactctcaccatcagcgacgtgcagtgtgacgatgctgccacttactactgtgcaggcggttatagtagtattagtgatactactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 14 - protein, 10 aa, synthetic construct
  MOD_RES 7: note = Phosphorylated Serine
CAPVEKXPAK

SEQ ID NO: 15 - protein, 113 aa, synthetic construct
QSVEESGGRLVTPGTPLTLTCTVSGIDLSSNVMMWVRQAPGKGLEYIGIITNSGIRYYASWAKGRFTISKTSTTVDLKITSPTTEDTATYFCARGAPNTGNIWGPGTLVTVSL

SEQ ID NO: 16 - protein, 109 aa, synthetic construct
QVLTQTASSVSAAVGGTVTINCQSSQSVYDNNYLSWYQQKPGQPPKLLIYQASKLASGVPSRFKGSGSGTQFTLTISDLECDDAVTYYCAGAYFGNIYTFGGGTEVVVK

SEQ ID NO: 17 - protein, 5 aa, synthetic construct
SNVMM

SEQ ID NO: 18 - protein, 16 aa, synthetic construct
IITNSGIRYYASWAKG

SEQ ID NO: 19 - protein, 8 aa, synthetic construct
GAPNTGNI

SEQ ID NO: 20 - protein, 13 aa, synthetic construct
QSSQSVYDNNYLS

SEQ ID NO: 21 - protein, 7 aa, synthetic construct
QASKLAS

SEQ ID NO: 22 - protein, 10 aa, synthetic construct
AGAYFGNIYT

SEQ ID NO: 23 - protein, 455 aa, synthetic construct
METGLRWLLLVAVLKGVQCQSVEESGGRLVTPGTPLTLTCTVSGIDLSSNVMMWVRQAPGKGLEYIGIITNSGIRYYASWAKGRFTISKTSTTVDLKITSPTTEDTATYFCARGAPNTGNIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 24 - protein, 236 aa, synthetic construct
MDTRAPTQLLGLLLLWLPGATFAQVLTQTASSVSAAVGGTVTINCQSSQSVYDNNYLSWYQQKPGQPPKLLIYQASKLASGVPSRFKGSGSGTQFTLTISDLECDDAVTYYCAGAYFGNIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 25 - DNA, 1368 bp, synthetic construct
  source 1..1368: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcaatgtaatgatgtgggtccgccaggctccagggaaggggctggaatacatcggaatcattactaatagtggtattagatactacgcgagctgggcgaaaggccgattcaccatctccaaaacctcgaccacagtggatctgaaaatcaccagtccgacaaccgaggacacggccacctatttctgtgccagaggggctcctaatactggtaacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 26 - protein, 711 aa, synthetic construct
ATGGACACGAGGGCCCCCACTCAGCTGCTGGGGCTCCTGCTGCTCTGGCTCCCAGGTGCCACATTTGCGCAAGTGCTGACCCAGACTGCATCGTCCGTGTCTGCAGCTGTGGGAGGCACAGTCACCATCAATTGCCAGTCCAGTCAGAGTGTTTATGATAACAACTACTTATCCTGGTATCAACAGAAACCAGGGCAGCCTCCCAAGCTCCTGATCTACCAGGCATCCAAACTGGCATCTGGGGTCCCATCGCGGTTCAAAGGCAGTGGATCTGGGACACAGTTCACTCTCACCATCAGCGACCTGGAGTGTGACGATGCTGTCACTTACTACTGTGCAGGCGCTTATTTTGGTAATATTTATACTTTCGGCGGAGGGACCGAGGTGGTGGTCAAAGGTGATCCAGTTGCACCTACTGTCCTCATCTTCCCACCAGCTGCTGATCAGGTGGCAACTGGAACAGTCACCATCGTGTGTGTGGCGAATAAATACTTTCCCGATGTCACCGTCACCTGGGAGGTGGATGGCACCACCCAAACAACTGGCATCGAGAACAGTAAAACACCGCAGAATTCTGCAGATTGTACCTACAACCTCAGCAGCACTCTGACACTGACCAGCACACAGTACAACAGCCACAAAGAGTACACCTGCAAGGTGACCCAGGGCACGACCTCAGTCGTCCAGAGCTTCAATAGGGGTGACTGTTAG

SEQ ID NO: 27 - protein, 12 aa, synthetic construct
  MOD_RES 5: note = Phosphorylated Threonine
CALAXPQKNGRV

SEQ ID NO: 28 - protein, 120 aa, synthetic construct
QSLEESGGGLVQPEGSLTLTCKASGFSFSSGYYMCWVRQAPGKGLEWSGCISARSGRTYYATWAKGRFTISKTSSTTVTLQVTSLTAADTATYFCARGNRFVSSSGDSMWGPGTLVTVSS

SEQ ID NO: 29 - protein, 113 aa, synthetic construct
DPVLTQTPSPVSAAVGGTVTINCQASQSVFSNNQLAWFQQKPGQPPKQLIYGASTLASGVSSRFKGSGYGTRFTLTISDVQCDDTATYYCLGEFTCSSVDCNAFGGGTEVVVE

SEQ ID NO: 30 - protein, 6 aa, synthetic construct
SGYYMC

SEQ ID NO: 31 - protein, 17 aa, synthetic construct
CISARSGRTYYATWAKG

SEQ ID NO: 32 - protein, 12 aa, synthetic construct
GNRFVSSSGDSM

SEQ ID NO: 33 - protein, 13 aa, synthetic construct
QASQSVFSNNQLA

SEQ ID NO: 34 - protein, 7 aa, synthetic construct
GASTLAS

SEQ ID NO: 35 - protein, 13 aa, synthetic construct
LGEFTCSSVDCNA

SEQ ID NO: 36 - protein, 462 aa, synthetic construct
METGLRWLLLVAVLKGVQCQSLEESGGGLVQPEGSLTLTCKASGFSFSSGYYMCWVRQAPGKGLEWSGCISARSGRTYYATWAKGRFTISKTSSTTVTLQVTSLTAADTATYFCARGNRFVSSSGDSMWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 37 - protein, 239 aa, synthetic construct
MDTRAPTQLLGLLLLWLPGAICDPVLTQTPSPVSAAVGGTVTINCQASQSVFSNNQLAWFQQKPGQPPKQLIYGASTLASGVSSRFKGSGYGTRFTLTISDVQCDDTATYYCLGEFTCSSVDCNAFGGGTEVVVEGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 38 - DNA, 1389 bp, synthetic construct
  source 1..1389: mol_type = other DNA
atggagactgggctgcgctggcttctcctggtcgctgtgctcaaaggtgtccagtgtcagtcgttggaggagtccgggggaggcctggtccagcctgagggatccctgacactcacctgcaaagcctctggattctccttcagtagcggctactacatgtgctgggtccgccaggctccagggaaggggctggagtggagcggttgcattagtgctcgtagtggtagaacttactacgcgacctgggcgaaaggccgattcaccatctccaaaacctcgtcgaccacggtgactctgcaagtgaccagtctgacagccgcggacacggccacctatttctgtgcgagagggaatagatttgttagtagtagtggtgattccatgtggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 39 - DNA, 720 bp, synthetic construct
  source 1..720: mol_type = other DNA
atggacacgagggcccccactcagctgctggggctcctgctgctctggctcccaggtgccatatgtgaccctgtgctgacccagactccatcccctgtgtctgcagctgtgggaggcacagtcaccatcaattgccaggccagtcagagtgtttttagtaacaaccaactagcctggtttcagcagaaaccagggcagcctcccaagcaactgatctatggtgcatccactctggcatctggggtctcatcgcggttcaaaggcagtggatatgggacacggttcactctcaccatcagcgacgtgcagtgtgacgatactgccacttactactgtctaggcgaatttacttgtagtagtgttgattgtaatgcttttggcggagggaccgaggtggtcgtcgagggtgatccagttgcacctactgtcctcatcttcccaccatctgctgatcttgtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 40 - protein, 14 aa, synthetic construct
  MOD_RES 8: note = Phosphorylated Serine
CLAGLPGXPKKRVR

SEQ ID NO: 41 - protein, 116 aa, synthetic construct
QSVEESGGRLVTPGTPLTLTCTVSGFSLSTYHMCWVRQAPGKGLEYIGMINRRAITSYASWAKGRFTISKTSTTVDLKITSPTTEDTATYFCARYSSGNDFDADIWGPGTLVTVSL

SEQ ID NO: 42 - protein, 112 aa, synthetic construct
AAVLTQTPSPVSAAVGGTVTISCQSSKSVYDRNLLSWFQQKPGQPPKLLIYKASTLASGVPSRFKGSGSGTQFTLTISDVQCDDAATYYCAGGYSGTSDAYPFGGGTEVVVK

SEQ ID NO: 43 - protein, 5 aa, synthetic construct
TYHMC

SEQ ID NO: 44 - protein, 16 aa, synthetic construct
MINRRAITSYASWAKG

SEQ ID NO: 45 - protein, 11 aa, synthetic construct
YSSGNDFDADI

SEQ ID NO: 46 - protein, 13 aa, synthetic construct
QSSKSVYDRNLLS

SEQ ID NO: 47 - protein, 7 aa, synthetic construct
KASTLAS

SEQ ID NO: 48 - protein, 12 aa, synthetic construct
AGGYSGTSDAYP

SEQ ID NO: 49 - protein, 458 aa, synthetic construct
METGLRWLLLVAVLKGVQCQSVEESGGRLVTPGTPLTLTCTVSGFSLSTYHMCWVRQAPGKGLEYIGMINRRAITSYASWAKGRFTISKTSTTVDLKITSPTTEDTATYFCARYSSGNDFDADIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 50 - protein, 238 aa, synthetic construct
MDTRAPTQLLGLLLLWLPGARCAAVLTQTPSPVSAAVGGTVTISCQSSKSVYDRNLLSWFQQKPGQPPKLLIYKASTLASGVPSRFKGSGSGTQFTLTISDVQCDDAATYYCAGGYSGTSDAYPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 51 - DNA, 1377 bp, synthetic construct
  source 1..1377: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctaagtacttaccacatgtgctgggtccgccaggctccagggaaggggctggaatacatcggaatgattaataggcgtgctattacatcctacgcgagctgggcgaaaggccgattcaccatctccaaaacctcgaccacggtggatctgaaaatcaccagtccgacaaccgaggacacggccacctatttctgtgccagatatagtagtggtaatgattttgatgcggacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 52 - DNA, 717 bp, synthetic construct
  source 1..717: mol_type = other DNA
atggacacgagggcccccactcagctgctggggctcctgctgctctggctcccaggtgccagatgtgccgccgtgctgacccagactccatctcccgtgtctgcagctgtgggaggcacagtcaccatcagttgccagtccagtaagagtgtttatgatagaaacctcttatcctggtttcagcagaaaccagggcagcctcccaagctcctgatctacaaggcttccactctggcatctggggtcccatcgcggttcaaaggcagtggatctgggacacagttcactctcaccatcagcgacgtgcagtgtgacgatgctgccacttactactgtgcaggcggttatagtggtactagtgatgcttatcctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 53 - protein, 12 aa, synthetic construct
  MOD_RES 8: note = Phosphorylated Threonine
CAPSKLWXPIKH

SEQ ID NO: 54 - protein, 114 aa, synthetic construct
QSLEESGGDLVKPGASLTLTCKGSGFSFTIRYNICWVRQAPGKGLEWIACVNSAYASWAKGRFTISKTSSTTVTLQMTSLTAADTATYFCVRYVDSRYYGVDLWGPGTLVTVSS

SEQ ID NO: 55 - protein, 114 aa, synthetic construct
QVLTQTPSSVSAALGGTVTISCQSSESVYKNNYLSWYQQKPGQPPKLLIYGASTLASGVPSRFKGSGSGTQFTLTISSVQCDDAATYYCQGGYRGDYSSGDGILFGGGTEVVVK

SEQ ID NO: 56 - protein, 6 aa, synthetic construct
IRYNIC

SEQ ID NO: 57 - protein, 12 aa, synthetic construct
CVNSAYASWAKG

SEQ ID NO: 58 - protein, 11 aa, synthetic construct
YVDSRYYGVDL

SEQ ID NO: 59 - protein, 13 aa, synthetic construct
QSSESVYKNNYLS

SEQ ID NO: 60 - protein, 15 aa, synthetic construct
QGGYRGDYSSGDGIL

SEQ ID NO: 61 - protein, 456 aa, synthetic construct
METGLRWLLLVAVLKGVLCQSLEESGGDLVKPGASLTLTCKGSGFSFTIRYNICWVRQAPGKGLEWIACVNSAYASWAKGRFTISKTSSTTVTLQMTSLTAADTATYFCVRYVDSRYYGVDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 62 - protein, 241 aa, synthetic construct
MDTRAPTQLLGLLLLWLPGATFAQVLTQTPSSVSAALGGTVTISCQSSESVYKNNYLSWYQQKPGQPPKLLIYGASTLASGVPSRFKGSGSGTQFTLTISSVQCDDAATYYCQGGYRGDYSSGDGILFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 63 - DNA, 1371 bp, synthetic construct
  source 1..1371: mol_type = other DNA
atggagactgggctgcgctggcttctcctggtcgctgtgctcaaaggtgtcctgtgtcagtcgttggaggagtccgggggagacctggtcaagcctggggcatccctgacactcacctgcaaaggctctggattctccttcactatcagatacaacatctgctgggtccgccaggctccagggaaggggctggagtggatcgcatgtgttaatagtgcctacgcgagctgggcgaaaggccgattcaccatctccaaaacctcgtcgaccacggtgactctgcaaatgaccagtctgacagccgcggacacggccacctatttctgtgtgagatatgttgatagtcgttactacggcgtggacctctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 64 - DNA, 726 bp, synthetic construct
  source 1..726: mol_type = other DNA
atggacacgagggcccccactcagctcctggggctcctgctgctctggctcccaggtgccacatttgcccaagtgctgacccagactccatcgtccgtgtctgcagctctgggaggcacagtcaccatcagttgccagtccagtgagagtgtttataagaacaactacttatcctggtatcaacagaaaccagggcagcctcccaagctcctgatctatggtgcatccactctggcatctggggtcccatcgcggttcaaaggcagtggatctgggacacagttcactctcaccatcagcagcgtgcagtgtgacgatgctgccacttactactgtcaaggcggttataggggcgattatagtagtggtgatggtattcttttcggcggagggaccgaggtggtcgtcaaaggtgatccagttgcacctactgtcctcatcttcccaccatctgctgatcttgtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 65 - protein, 323 aa, synthetic construct
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 66 - protein, 104 aa, synthetic construct
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 67 - protein, 104 aa, synthetic construct
GDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 68 - DNA, 16 bp, synthetic construct
  source 1..16: mol_type = other DNA
tccggactctagcacc

SEQ ID NO: 69 - DNA, 19 bp, synthetic construct
  source 1..19: mol_type = other DNA
gcgctgtgccggcgatatc